(19) United States
(12) Patent Application Publication
(10) Pub. No.: US 2008/0255144 A1
(43) Pub. Date: Oct. 16, 2008

(54) Acylated Piperidines as Glycine Transporter Inhibitors

(75) Inventors: Daniel Marcus Bradley, Essex (GB); Clive Leslie Branch, Essex (GB); Wai Ngor Chan, Essex (GB); Steven Coulton, Essex (GB); Anthony William Dean, Hertfordshire (GB); Paul Martin Doyle, Essex (GB); Brian Evans, Hertfordshire (GB); Martin Leonard Gilpin, Essex (GB); Sharon Lisa Gough, Hertfordshire (GB); Jacqueline Anne Macritchie, Essex (GB); Howard Robert Marshall, Essex (GB); David John Nash, Essex (GB); Roderick Alan Porter, Essex (GB); Simon Edward Ward, Middlesex (GB)

Correspondence Address: SMITHKLINE BEECHAM CORPORATION;CORPORATE INTELLECTUAL PROPERTY-US, UW2220, P. O. BOX 1539, KING OF PRUSSIA, PA, 19406-0939, US

(73) Assignee: Glaxo Group Limited

(21) Appl. No.: 11/908,151

(22) PCT Filed: Mar. 9, 2006
(86) PCT No.: PCT/EP06/02484

§ 371 Date: Sep. 10, 2007

(30) Foreign Application Priority Data
Mar. 11, 2005 (GB) 0505084.4

(51) Int. Cl. A61K 31/496; C07D 241/04; C07D 405/10; C07D 401/10; A61P 25/18; C07D 401/04; A61K 31/495

(52) U.S. Cl. 514/253.04; 544/391; 544/379; 544/360; 544/362; 514/255.01; 514/254.1; 514/253.01

(57) ABSTRACT

The invention provides a compound of formula (I) or a salt or solvate thereof:

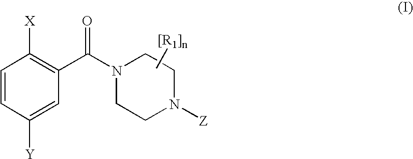

wherein R¹, n, X, Y and Z are as defined in the specification, and uses of such compounds. The compounds inhibit GlyT1 transporters and are useful in the treatment of certain neurological and neuropsychiatric disorders, including schizophrenia.

The present invention relates to glycine transporter inhibiting compounds, their use in the manufacture of medicaments for treating neurological and neuropsychiatric disorders, in particular psychoses, dementia or attention deficit disorder. The invention further comprises processes to make these compounds and pharmaceutical formulations thereof.

Molecular cloning has revealed the existence in mammalian brains of two classes of glycine transporters, termed GlyT1 and GlyT2. GlyT1 is found predominantly in the forebrain and its distribution corresponds to that of glycinergic pathways and NMDA receptors (Smith, et al., Neuron, 8, 1992: 927-935). Molecular cloning has further revealed the existence of three variants of GlyT1, termed GlyT-1a, GlyT-1b and GlyT-1c (Kim et al., Molecular Pharmacology, 45, 1994: 608-617), each of which displays a unique distribution in the brain and peripheral tissues. The variants arise by differential splicing and exon usage, and differ in their N-terminal regions. GlyT2, in contrast, is found predominantly in the brain stem and spinal cord, and its distribution corresponds closely to that of strychnine-sensitive glycine receptors (Liu et al., J. Biological Chemistry, 268, 1993: 22802-22808; Jursky and Nelson, J. Neurochemistry, 64, 1995: 1026-1033). Another distinguishing feature of glycine transport mediated by GlyT2 is that it is not inhibited by sarcosine as is the case for glycine transport mediated by GlyT1. These data are consistent with the view that, by regulating the synaptic levels of glycine, GlyT1 and GlyT2 selectively influence the activity of NMDA receptors and strychnine-sensitive glycine receptors, respectively.

NMDA receptors are critically involved in memory and learning (Rison and Staunton, Neurosci. Biobehav. Rev. 19 533-552 (1995); Danysz et al, Behavioral Pharmacol., 6 455-474 (1995)); and, furthermore, decreased function of NMDA-mediated neurotransmission appears to underlie, or contribute to, the symptoms of schizophrenia (Olney and Farber, Archives General Psychiatry, 52, 998-1007 (1996). Thus, agents that inhibit GlyT1 and thereby increase glycine activation of NMDA receptors can be used as novel antipsychotics and anti-dementia agents, and to treat other diseases in which cognitive processes are impaired, such as attention deficit disorders and organic brain syndromes. Conversely, over-activation of NMDA receptors has been implicated in a number of disease states, in particular the neuronal death associated with stroke and possibly neurodegenerative diseases, such as Alzheimer's disease, multi-infarct dementia, AIDS dementia, Huntington's disease, Parkinson's disease, amyotrophic lateral sclerosis or other conditions in which neuronal cell death occurs, such as stroke or head trauma. Coyle & Puttfarcken, Science, 262, 689-695 (1993); Lipton and Rosenberg, New Engl. J. of Medicine, 330, 613-622 (1993); Choi, Neuron, 1, 623-634 (1988). Thus, pharmacological agents that increase the activity of GlyT1 will result in decreased glycine-activation of NMDA receptors, which activity can be used to treat these and related disease states. Similarly, drugs that directly block the glycine site of the NMDA receptors can be used to treat these and related disease states.

Glycine transport inhibitors are already known in the art, for example as disclosed in published international patent application WO03/055478 (SmithKline Beecham).

However, there still remains the need to identify further compounds that can inhibit GlyT1 transporters, including those that inhibit GlyT1 transporters selectively over GlyT2 transporters.

International patent application WO97/28128 (Zeneca Limited) discloses certain pyridinyl, pyridazinyl, pyrimidinyl and triazinyl derivatives which are claimed to inhibit the enzyme oxido squalene cyclase. European patent application EP1247809 (Pfizer Products Inc) discloses certain triazine derivatives as being useful as sorbitol dehydrogenase inhibitors.

It has now been found that a novel class of compounds inhibit GlyT1 transporters and are thus useful in the treatment of certain neurological and neuropsychiatric disorders, including schizophrenia.

Thus, in a first aspect, there is provided a compound of formula (I) or a salt or solvate thereof:

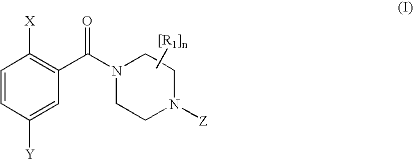

wherein
X is selected from C₅₋₁₁aryl and C₄₋₁₀heteroaryl, said C₅₋₁₁aryl and C₄₋₁₀heteroaryl being optionally substituted with one or more groups selected from halogen, hydroxy, cyano, C₁₋₄alkyl, C₁₋₄alkoxy, haloC₁₋₄alkyl, haloC₁₋₄alkoxy and C₁₋₄alkylthio.
Y is —S(O)ₘR₂ or —SO₂NR₃R₄ wherein
   m is 1 or 2; and
   R₂ is selected from C₁₋₆alkyl, C₃₋₇cycloalkyl, C₅₋₁₁aryl and C₄₋₁₀heteroaryl, where the C₁₋₆alkyl, C₃₋₇cycloalkyl, C₅₋₁₁aryl or C₄₋₁₀heteroaryl groups are optionally substituted with one or two groups selected from halo, C₁₋₄alkoxy and C₁₋₄haloalkoxy;
   R₃ and R₄ are independently selected from hydrogen and C₁₋₆alkyl, where the C₁₋₆alkyl is optionally substituted with one or more groups selected from halo, C₁₋₄alkoxy and C₁₋₄haloalkoxy;
n is 0, 1 or 2,
each R₁ is independently selected from C₁₋₆alkyl and C₁₋₆haloalkyl; and
Z is:
   an optionally substituted phenyl Z′:

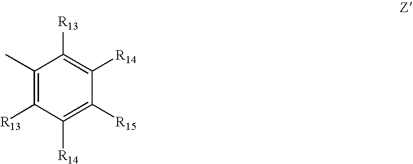

wherein each R₁₃ is independently selected from hydrogen, halogen, hydroxy, cyano, amino, C₂₋₆alkyl, C₁₋₄alkoxy, haloC₁₋₄alkyl, haloC₁₋₄alkoxy, C₆₋₁₁arylC₁₋₄alkoxy, C₁₋₄alkylthio, C₁₋₄alkoxyC₁₋₄alkyl, C₁₋₄haloalkoxyC₁₋₄alkyl, halohydroxyC₁₋₄alkyl, C₁₋₄alkoxyC₁₋₄alkoxyC₁₋₄alkyl, C₁₋₄alkoxyhaloC₁₋₄alkyl, C₃₋₆cycloalkylC₁₋₄alkyl, C₃₋₆cycloalkylC₁₋₄alkyl substituted by one or more C₁₋₄alkoxy groups in the C₁₋₄alkyl portion, C₃₋₆cycloalkyl, C₃₋₆cycloalkylC₁₋₄alkoxy, C₃₋₆cycloalkylhydroxyC₁₋₄alkyl, C₃₋₆cycloalkylC₁₋₄acyl, C₃₋₆cycloalkylC₁₋₄alkoxyC₁₋₄alkyl, C₃₋₆cycloalkoxyC₁₋₄alkyl, C₁₋₄alkanoyl, C₁₋₄haloalkanoyl, C₁₋₄alkoxycarbonyl, C₁₋₄alkoxycarbonylC₁₋₄alkyl, C₁₋₄alkylsulfonyl, C₁₋₄haloalkylsulfonyl, C₁₋₄alkylsulfinyl, C₁₋₄haloalkylsulfinyl, C₁₋₄alkylsulfonyloxy, C₁₋₄alkylsulfonylC₁₋₄alkyl, C₆₋₁₁arylsulfonyl, C₆₋₁₁arylsulfonyloxy, C₆₋₁₁arylsulfonylC₁₋₄alkyl, C₁₋₄alkylsulfonamido, C₄₋₉heteroarylsulfonyl, C₁₋₄alkylsulfonamidoC₁₋₄alkyl, C₁₋₄alkylamidoC₁₋₄alkyl, C₆₋₁₁arylsulfonamido, C₆₋₁₁arylcarboxamido, C₆₋₁₁arylsulfonamidoC₁₋₄alkyl, C₆₋₁₁arylcarboxamidoC₁₋₄alkyl, C₆₋₁₁aroyl, C₆₋₁₁aroylC₁₋₄alkyl, C₆₋₁₁arylC₁₋₄alkanoyl, formyl, C₁₋₄acyl, haloC₁₋₄acyl, arylC₁₋₄alkoxyC₁₋₄alkyl, C₆₋₁₁aryl, C₆₋₁₁aryl substituted by one to three groups selected from C₁₋₄alkyl, hydroxy, halogen, C₁₋₄alkoxy, C₁₋₄acyl and trifluoromethyl; C₆₋₁₁arylC₁₋₄alkyl, C₄₋₁₀heteroaryl, C₄₋₁₀heteroaryl substituted by one to three C₁₋₄alkyl groups, C₁₋₄alkylaminoC₁₋₄alkyl, a group —NR_{9″}R_{10″}, —(CH₂)ₚCONR₉R₁₀, —(CH₂)ₚSO₂NR₉R₁₀ or —(CH₂)ₚNR₉SO₂R₁₀, —CR_{9′}═NR_{10′}, —CR_{9′}═NOR_{10′}, —CR_{9′}═C(CN)₂, —CR_{9′}═CH(CN), —(CH₂)_{q}NR_{9′}R_{10′} and —O(CH₂)_{q}NR_{9′}R_{10′} wherein
each R₉ and R₁₀ is independently C₁₋₄alkyl, or where appropriate R₉R₁₀ forms part of a C₃₋₆azacyloalkane or C₃₋₆(2-, 3- or 4-oxo)azacycloalkane ring
each R_{9′} and R_{10′} is independently selected from R₉ and R₁₀ and hydrogen;
each R_{9″} and R_{10″} is independently selected from R_{9′} and R_{10′} and C₁₋₄alkanoyl;
p is selected from 0, 1, 2, 3 or 4;
q is selected from 2, 3 or 4;
   wherein each R₁₄ is independently selected from hydrogen, halogen, hydroxy, cyano, amino, nitro, C₁₋₆alkyl, C₁₋₄alkoxy, haloC₁₋₄alkyl, haloC₁₋₄alkoxy, C₆₋₁₁arylC₁₋₄alkoxy, C₁₋₄alkylthio, hydroxyC₁₋₄alkyl, C₁₋₄haloalkoxyC₁₋₄alkyl, halohydroxyC₁₋₄alkyl, C₁₋₄alkoxyC₁₋₄alkoxyC₁₋₄alkyl, C₁₋₄alkoxyhaloC₁₋₄alkyl, C₃₋₆cycloalkylC₁₋₄alkyl, C₃₋₆cycloalkylC₁₋₄alkyl substituted by one or more C₁₋₄alkoxy groups in the C₁₋₄alkyl portion, C₃₋₆cycloalkyl, C₃₋₆cycloalkylC₁₋₄alkoxy, C₃₋₆cycloalkylhydroxyC₁₋₄alkyl, C₃₋₆cycloalkylC₁₋₄acyl, C₃₋₆cycloalkylC₁₋₄alkoxyC₁₋₄alkyl, C₃₋₆cycloalkoxyC₁₋₄alkyl, C₁₋₄alkanoyl, C₁₋₄haloalkanoyl, C₁₋₄alkoxycarbonyl, C₁₋₄alkoxycarbonylC₁₋₄alkyl, C₁₋₄alkylsulfonyl, C₁₋₄haloalkylsulfonyl, C₁₋₄alkylsulfinyl, C₁₋₄haloalkylsulfinyl, C₁₋₄alkylsulfonyloxy, C₁₋₄alkylsulfonylC₁₋₄alkyl, C₆₋₁₁arylsulfonyl, C₆₋₁₁arylsulfonyloxy, C₆₋₁₁arylsulfonylC₁₋₄alkyl, C₁₋₄alkylsulfonamido, C₄₋₉heteroarylsulfonyl, C₁₋₄alkylsulfonamidoC₁₋₄alkyl, C₆₋₁₁ arylsulfonamidoC₁₋₄alkyl, C₆₋₁₁aroyl, C₆₋₁₁aroylC₁₋₄alkyl, C₆₋₁₁arylC₁₋₄alkanoyl, formyl, C₁₋₄acyl, haloC₁₋₄acyl, arylC₁₋₄alkoxyC₁₋₄alkyl, C₆₋₁₁aryl, C₆₋₁₁aryl substituted by one to three groups selected from C₁₋₄alkyl, hydroxy, halogen, C₁₋₄alkoxy, C₁₋₄acyl and trifluoromethyl; C₆₋₁₁arylC₁₋₄alkyl, C₄₋₁₀heteroaryl, C₄₋₁₀heteroaryl substituted by one to three C₁₋₄alkyl groups, C₁₋₄alkylaminoC₁₋₄alkyl, a group —NR_{9″}R_{10″}, —(CH₂)ₚSO₂NR_{9′}R_{10′} or —(CH₂)ₚNR_{10′}SO₂R_{9′}, —CR_{9′}═NR_{10′}, —CR_{9′}═NOR_{10′}, —CR_{9′}═C(CN)₂, —CR_{9′}═CH(CN), —(CH₂)_{q}NR_{9′}R_{10′} and —O(CH₂)_{q}NR_{9′}R_{10′}, wherein
each R₉ and R₁₀ is independently C₁₋₄alkyl, or where appropriate R₉R₁₀ forms part of a C₃₋₆azacyloalkane or C₃₋₆(2-, 3- or 4-oxo)azacycloalkane ring
each R_{9′} and R_{10′} is independently selected from R₉ and R₁₀ and hydrogen;
each R_{9″} and R_{10″} is independently selected from R_{9′} and R_{10′} and C₁₋₄alkanoyl;
p is selected from 0, 1, 2, 3 or 4;
q is selected from 2, 3 or 4;
   wherein R₁₅ is selected from hydrogen, halogen, hydroxy, cyano, nitro, C₁₋₆alkyl, C₂₋₄alkenyl, C₁₋₄alkoxy, haloC₁₋₄alkyl, haloC₁₋₄alkoxy, C₆₋₁₁arylC₁₋₄alkoxy, C₁₋₄alkylthio, hydroxyC₁₋₄alkyl, cyanoC₁₋₄alkyl, C₁₋₄alkoxyC₁₋₄alkyl, C₁₋₄haloalkoxyC₁₋₄alkyl, C₁₋₄alkoxyhaloC₁₋₄alkyl, halohydroxyC₁₋₄alkyl, C₁₋₄alkoxyC₁₋₄alkoxyC₁₋₄alkyl, haloC₁₋₄alkoxyC₁₋₄alkoxyC₁₋₄alkyl, C₁₋₄alkoxyC₁₋₄haloalkoxyC₁₋₄alkyl, C₁₋₄alkoxyC₁₋₄alkoxyC₁₋₄haloalkyl C₁₋₄alkoxyC₁₋₄alkoxy, C₁₋₄alkoxyhaloC₁₋₄alkyl, C₃₋₆cycloalkylC₁₋₄alkyl, C₃₋₆cycloalkylC₁₋₄alkyl substituted by one or more C₁₋₄alkoxy groups in the C₁₋₄alkyl portion, C₃₋₆cycloalkyl, C₃₋₆cycloalkylC₁₋₄alkoxy, C₃₋₆cycloalkylhydroxyC₁₋₄alkyl, C₃₋₆cycloalkylC₁₋₄acyl, C₃₋₆cycloalkylC₁₋₄alkoxyC₁₋₄alkyl, C₃₋₆cycloalkoxyC₁₋₄alkyl, C₁₋₄alkanoyl, C₁₋₄haloalkanoyl, C₁₋₄alkoxycarbonyl, C₁₋₄alkoxycarbonylC₁₋₄alkyl, C₁₋₄alkylsulfonyl, C₁₋₄haloalkylsulfonyl, C₁₋₄alkylsulfinyl, C₁₋₄haloalkylsulfinyl, C₁₋₄alkylsulfonyloxy, C₁₋₄alkylsulfonylC₁₋₄alkyl, C₆₋₁₁arylsulfonyl, C₆₋₁₁arylsulfonyloxy, C₆₋₁₁arylsulfonylC₁₋₄alkyl, C₁₋₄alkylsulfonamido, C₄₋₉heteroarylsulfonyl, C₁₋₄alkylsulfonamidoC₁₋₄alkyl, C₁₋₄alkylamidoC₁₋₄alkyl, C₆₋₁₁arylsulfonamido, C₆₋₁₁arylcarboxamido, C₆₋₁₁arylsulfonamidoC₁₋₄alkyl, C₆₋₁₁arylcarboxamidoC₁₋₄alkyl, C₆₋₁₁aroyl, C₆₋₁₁aroylC₁₋₄alkyl, C₆₋₁₁arylC₁₋₄alkanoyl, formyl, C₁₋₄acyl, haloC₁₋₄acyl, arylC₁₋₄alkoxyC₁₋₄alkyl, C₆₋₁₁aryl, C₆₋₁₁aryl substituted by one to three groups selected from C₁₋₄alkyl, hydroxy, halogen, C₁₋₄alkoxy, C₁₋₄acyl and trifluoromethyl; C₆₋₁₁ arylC₁₋₄alkyl, C₃₋₆heterocyclyl, C₃₋₁₀heteroaryl, C₃₋₁₀heteroaryl substituted by one to three C₁₋₄alkyl groups, C₁₋₄alkylaminoC₁₋₄alkyl, a group —NR₉R₁₀, —(CH₂)ₚSO₂NR_{9′}R_{10′}, —NR₉C(O)R₁₀, —NHC(O)—C₁₋₂alkyl, —(CH₂)ₚNR_{10′}SO₂R_{9′}, —CR_{9′}═NR_{10′}, —CR_{9′}═NOR_{10′}, —CR_{9′}═C(CN)₂, —CR_{9′}═CH(CN), —(CH₂)ᵣN(R_{9′})C(O)R_{10′}, (CH₂)ᵣNR₉R₁₀ and —O(CH₂)_{q}NR_{9′}R_{10′}, wherein
each R₉ and R₁₀ is independently halo C₁₋₄alkyl, C₁₋₄alkyl, C₅₋₁₀aryl, or where appropriate R₉R₁₀ forms part of a C₃₋₆azacyloalkane ring
each R_{9′} and R_{10′} is independently selected from R₉ and R₁₀ and hydrogen;
each R_{9″} and R_{10″} is independently selected from R_{9′} and R_{10′}, C₁₋₄acyl, haloC₁₋₄acyl, haloaryl and C₁₋₄alkanoyl;
p is selected from 0, 1, 2, 3 or 4;
q is selected from 2, 3 or 4;
r is selected from 1, 2, 3 or 4;
or Z is selected from:
   a monocyclic or bicyclic heteroaryl group, or a bicyclic C₈₋₁₁aryl group which heteroaryl or aryl group is optionally substituted by one or more groups selected from halogen, hydroxy, oxo, cyano, amino, nitro, C₁₋₆alkyl, C₁₋₄alkoxy, haloC₁₋₄alkyl, haloC₁₋₄alkoxy, C₆₋₁₁arylC₁₋₄alkoxy, C₁₋₄alkylthio, hydroxyC₁₋₄alkyl, C₁₋₄alkoxyC₁₋₄alkyl, C₁₋₄haloalkoxyC₁₋₄alkyl, halohydroxyC₁₋₄alkyl, C₁₋₄alkoxyC₁₋₄alkoxyC₁₋₄alkyl, C₁₋₄alkoxyhaloC₁₋₄alkyl, C₃₋₆cycloalkylC₁₋₄alkyl, C₃₋₆cycloalkylC₁₋₄alkyl substituted by one or more C₁₋₄alkoxy groups in the C₁₋₄alkyl portion, C₃₋₆cycloalkyl, C₃₋₆cycloalkylC₁₋₄alkoxy, C₃₋₆cycloalkylhydroxyC₁₋₄alkyl, C₃₋₆cycloalkylC₁₋₄acyl, C₃₋₆cycloalkylC₁₋₄alkoxyC₁₋₄alkyl, C₃₋₆cycloalkoxyC₁₋₄alkyl, C₁₋₄alkanoyl, C₁₋₄haloalkanoyl, C₁₋₄alkoxycarbonyl, C₁₋₄alkoxycarbonylC₁₋₄alkyl, C₁₋₄alkylsulfonyl, C₁₋₄haloalkylsulfonyl, C₁₋₄alkylsulfinyl, C₁₋₄haloalkylsulfinyl, C₁₋₄alkylsulfonyloxy, C₁₋₄alkylsulfonylC₁₋₄alkyl, C₆₋₁₁ arylsulfonyl, C₆₋₁₁arylsulfonyloxy, C₆₋₁₁arylsulfonylC₁₋₄alkyl, C₁₋₄alkylsulfonamido, C₄₋₉heteroarylsulfonyl, C₁₋₄alkylsulfonamidoC₁₋₄alkyl, C₁₋₄alkylamidoC₁₋₄alkyl, C₆₋₁₁arylsulfonamido, C₆₋₁₁arylcarboxamido, C₆₋₁₁ arylsulfonamidoC₁₋₄alkyl, C₆₋₁₁arylcarboxamidoC₁₋₄alkyl, C₆₋₁₁aroyl, C₆₋₁₁aroylC₁₋₄alkyl, C₆₋₁₁arylC₁₋₄alkanoyl, formyl, C₁₋₄acyl, haloC₁₋₄acyl, arylC₁₋₄alkoxyC₁₋₄alkyl, C₆₋₁₁aryl, C₆₋₁₁aryl substituted by one to three groups selected from C₁₋₄alkyl, hydroxy, halogen, C₁₋₄alkoxy, C₁₋₄acyl and trifluoromethyl; C₆₋₁₁arylC₁₋₄alkyl, C₄₋₁₀heteroaryl, C₄₋₁₀heteroaryl substituted by one to three C₁₋₄alkyl groups, C₁₋₄alkylaminoC₁₋₄alkyl, a group —NR_{9″}R_{10″}, —(CH₂)ₚCONR₉R₁₀, —(CH₂)ₚSO₂NR_{9′}R_{10′} or —(CH₂)ₚNR_{10′}SO₂R₉, —CR_{9′}═NR_{10′}, —CR_{9′}═NOR_{10′}, —CR_{9′}═C(CN)₂, —CR_{9′}═CH(CN), —(CH₂)_{q}NR_{9′}R_{10′} and —O(CH₂)_{q}NR_{9′}R_{10′} wherein
each R₉ and R₁₀ is independently C₁₋₄alkyl, C₅₋₁₀aryl, or where appropriate R₉R₁₀ forms part of a C₃₋₆azacyloalkane or C₃₋₆(2-, 3- or 4-oxo)azacycloalkane ring
each R_{9′} and R_{10′} is independently selected from R₉ and R₁₀ and hydrogen;
each R_{9″} and R_{10″} is independently selected from R_{9′} and R_{10′} and C₁₋₄alkanoyl;
p is selected from 0, 1, 2, 3 or 4; and
q is selected from 2, 3 or 4.

As used herein, the term “C₁₋₆alkyl” refers to a straight or branched alkyl which contains from one to six carbon atoms in all isomeric forms. Examples include methyl, ethyl, propyl, isopropyl, butyl, isobutyl, sec-butyl, tert-butyl, pentyl, neopentyl, sec-pentyl, n-pentyl, isopentyl, tert-pentyl and hexyl.

As used herein, the term “C₃₋₇cycloalkyl” refers to a non-aromatic cyclic saturated hydrocarbon ring having from three to seven carbon atoms. Examples include cyclopropyl, cyclobutyl, cyclopentyl, cyclohexyl and cycloheptyl. The term “C₃₋₆cycloalkyl” refers to a non-aromatic cyclic saturated hydrocarbon ring having from three to six carbon atoms. Examples include cyclopropyl, cyclobutyl, cyclopentyl and cyclohexyl.

As used herein, the term “C₁₋₄alkylene” refers to a straight or branched chain divalent hydrocarbon radical, which contains 1, 2, 3 or 4 carbon atoms. Examples include methylene, ethylene, n-propylene and n-butylene.

As used herein, the term “aryl” preferably refers to phenyl or a 8- to 11-membered bicyclic aromatic group in which at least one of the rings is aromatic. Examples of 8- to 11-membered bicyclic aromatic groups include indenyl, azulenyl, naphthyl and tetrahydronaphthyl.

As used herein, the terms “heteroaryl” and “heteroaromatic group” preferably refer to a 5- or 6-membered monocyclic aromatic group wherein one, two or three carbon atoms are replaced by a heteroatom independently selected from N, O and S, or to a 8- to 11-membered bicyclic aromatic group in which at least one of the rings is aromatic and wherein one to four carbon atoms in total are replaced by a heteroatom independently selected from N, O and S. Examples of 5- or 6-membered monocyclic heteroaromatic groups include furanyl, thienyl, pyrrolyl, oxazolyl, thiazolyl, imidazolyl, oxadiazolyl, thiadiazolyl, pyridyl, triazolyl, triazinyl, pyridazyl, pyrimidinyl, isothiazolyl, isoxazolyl, pyrazinyl, pyrazolyl and pyrimidinyl; examples of 8- to 11-membered bicyclic heteroaromatic groups include quinoxalinyl, quinazolinyl, pyridopyrazinyl, benzoxazolyl, benzothiophenyl, benzimidazolyl, naphthyridinyl, quinolinyl, benzofuranyl, indolyl, benzothiazolyl, oxazolyl[4,5-b]pyridyl, pyridopyrimidinyl, isoquinolinyl and pyrrolopyridinyl, for example 1H-indolyl, 1H-pyrrolo[2,3-b]pyridyl.

As used herein, the terms “halogen” and its abbreviation “hal” refer to fluorine, chlorine, bromine, or iodine.

As used herein, the term “salt” refers to any salt of a compound according to the present invention prepared from an inorganic or organic acid or base, quaternary ammonium salts and internally formed salts. Physiologically acceptable salts are particularly suitable for medical applications because of their greater aqueous solubility relative to the parent compounds. Such salts must clearly have a physiologically acceptable anion or cation. Suitable physiologically acceptable salts of the compounds of the present invention include acid addition salts formed with inorganic acids such as hydrochloric, hydrobromic, hydroiodic, phosphoric, metaphosphoric, nitric and sulfuric acids, and with organic acids, such as tartaric, acetic, trifluoroacetic, citric, malic, lactic, fumaric, benzoic, formic, propionic, glycolic, gluconic, maleic, succinic, camphorsulfuric, isothionic, mucic, gentisic, isonicotinic, saccharic, glucuronic, furoic, glutamic, ascorbic, anthranilic, salicylic, phenylacetic, mandelic, embonic (pamoic), methanesulfonic, ethanesulfonic, pantothenic, stearic, sulfanilic, alginic, galacturonic and arylsulfonic, for example benzenesulfonic and p-toluenesulfonic, acids; base addition salts formed with alkali metals and alkaline earth metals and organic bases such as N,N-dibenzylethylenediamine, chloroprocaine, choline, diethanolamine, ethylenediamine, meglumaine (N-methylglucamine), lysine and procaine; and internally formed salts. Salts having a non-physiologically acceptable anion or cation are within the scope of the invention as useful intermediates for the preparation of physiologically acceptable salts and/or for use in non-therapeutic, for example, in vitro, situations.

As used herein, the term “solvate” refers to a complex of variable stoichiometry formed by a solute (in this invention, a compound of formula (I) or a salt thereof) and a solvent. Such solvents for the purpose of the invention may not interfere with the biological activity of the solute. Examples of suitable solvents include, but are not limited to, water, methanol, ethanol and acetic acid. Preferably the solvent used is a pharmaceutically acceptable solvent. Examples of suitable pharmaceutically acceptable solvents include water, ethanol and acetic acid. Most preferably the solvent used is water.

In formula (I) above, X may, for example, be phenyl, furanyl, thiophenyl or pyridinyl, each of which is optionally substituted by one or two groups selected from the group consisting of halogen, C₁₋₆alkoxy and cyano. Thiophene and furanyl groups may be connected at the 2- or 3-position; pyridine groups may be attached at the 2-, 3- or 4-position. Preferably X is optionally substituted phenyl. Preferred substituents include fluoro, for example in the 4-, 3- or 2-positions. There may be one or more than one substituents. Most preferably X is phenyl substituted with one or two fluorine atoms, most preferably one fluorine atom.

In another embodiment, X is a 8- to 11-membered bicyclic aromatic group such as indenyl, azulenyl, naphthyl or tetrahydronaphthyl, which may optionally be substituted. Where only one of the rings is aromatic (such as in tetrahydronaphthyl), X is, for example, attached to the phenyl ring in formula (I) via the aromatic ring.

In an embodiment, X is selected from C₅₋₁₁aryl and C₄₋₁₀heteroaryl, said C₅₋₁₁aryl and C₄₋₁₀heteroaryl being substituted with one or more groups selected from cyano, haloC₁₋₄alkyl, haloC₁₋₄alkoxy and C₁₋₄alkylthio, and optionally with one or more further groups as set out above.

In a preferred embodiment, Y is S(O)ₘR₂ wherein m is 1 or 2 and R₂ is as defined above. More preferably Y is S(O)ₘR₂ wherein m is 2 and R₂ is as defined above.

In one embodiment, R₂ is C₁₋₆alkyl which may be optionally substituted by one two or three groups selected from the group consisting of halogen, C₁₋₄alkoxy and haloC₁₋₄alkoxy. When R₂ is optionally substituted heteroaryl, the heteroaryl group may be joined to the —S(O)ₘ— moiety through any suitable atom in the heteroaryl group. Most preferably, R₂ is methyl. In one embodiment, Y is C₁₋₆alkysulfonyl, for example as —SO₂CH₃ or —SO₂C₂H₅.

In one embodiment, n is 0.

In another embodiment, n is 1 or 2 and R₁ is C₁₋₄alkyl.

In one embodiment, Z is selected from the group consisting of phenyl group Z′ as described herein, a 8- to 11-membered bicyclic aromatic group, a 5- or 6-membered monocyclic heteroaromatic group or a 8- to 11-membered bicyclic heteroaromatic group. In one embodiment, Z is selected from the group consisting of phenyl Z′ as described herein, pyridyl, pyrimidinyl, pyridazinyl, pyrazinyl, triazolyl, triazinyl, pyrrolyl, 1H-pyrrolo[2,3-b]pyridinyl, imidazolyl, thienyl, furanyl, thiadiazolyl, isoxazolyl, isothiazolyl, thiazolyl, oxadiazolyl and oxazolyl, benzothiazolyl, 1,4-benzodioxinyl, 2,3-dihydro-1,4-benzodioxinyl, benzoxazolyl, indolyl, quinolyl, isoquinolinyl, 1-benzopyranyl, 2-benzopyranyl, dihydro-1-benzopyranyl, dihydro-2-benzopyranyl, quinoxalinyl and quinazolinyl, each of which is optionally substituted by one to three groups as described above.

In one embodiment, Z is selected from the group consisting of phenyl Z′ as described herein, 2-pyridyl, 3-pyridyl, 2-pyrimidinyl, 5-pyrimidinyl, 3-pyridazinyl, pyrazinyl, 1,2,3-triazolyl, 1,2,4-triazolyl, pyrrolyl, 1H-pyrrolo[2,3-b]pyridinyl, imidazolyl, thienyl, furanyl, thiadiazolyl, isoxazolyl, isothiazolyl, thiazolyl, oxadiazolyl and oxazolyl, benzothiazolyl, 1,4-benzodioxinyl, 2,3-dihydro-1,4-benzodioxinyl, benzoxazolyl, indolyl, quinolyl, isoquinolinyl, 1-benzopyranyl, 2-benzopyranyl, dihydro-1-benzopyranyl, dihydro-2-benzopyranyl, quinoxalinyl and quinazolinyl, each of which is optionally substituted as described above.

In one embodiment, Z is a phenyl group Z′:

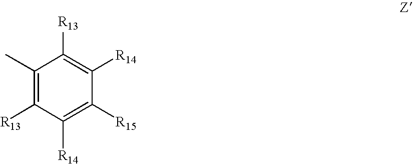

wherein each R₁₃ is independently selected from hydrogen, halogen, formyl and C₁₋₄acyl,
wherein each R₁₄ is independently selected from hydrogen, halogen, C₁₋₆alkyl, C₁₋₄alkoxy, haloC₁₋₄alkyl, haloC₁₋₄alkoxy and —NR_{9″}R_{10″}, wherein R_{9″}R_{10″} forms part of a C₃₋₆azacyloalkane or C₃₋₆(2-, 3- or 4-oxo)azacycloalkane ring;
wherein R₁₅ is selected from hydrogen, halogen, hydroxy, cyano, nitro, C₁₋₆alkyl, C₂₋₄alkenyl, C₁₋₄alkoxy, haloC₁₋₄alkyl, haloC₁₋₄alkoxy, C₆₋₁₁arylC₁₋₄alkoxy, C₁₋₄alkylthio, hydroxyC₁₋₄alkyl, cyanoC₁₋₄alkyl, C₁₋₄alkoxyC₁₋₄alkyl, C₁₋₄haloalkoxyC₁₋₄alkyl, C₁₋₄alkoxyhaloC₁₋₄alkyl, C₁₋₄alkanoyl, haloC₁₋₄alkanoyl, halohydroxyC₁₋₄alkyl, C₁₋₄alkoxyC₁₋₄alkoxyC₁₋₄alkyl, C₁₋₄alkoxyC₁₋₄alkoxy, C₁₋₄alkoxyhaloC₁₋₄alkyl, haloC₁₋₄alkoxyC₁₋₄alkoxyC₁₋₄alkyl, C₁₋₄alkoxyC₁₋₄haloalkoxyC₁₋₄alkyl, C₁₋₄alkoxyC₁₋₄alkoxyC₁₋₄haloalkyl, C₃₋₆cycloalkylC₁₋₄alkyl substituted by one or more C₁₋₄alkoxy groups in the C₁₋₄alkyl portion, C₃₋₆cycloalkylC₁₋₄alkoxy, C₃₋₆cycloalkylhydroxyC₁₋₄alkyl, C₃₋₆cycloalkylC₁₋₄alkoxyC₁₋₄alkyl, C₃₋₆cycloalkoxyC₁₋₄alkyl, C₁₋₄alkoxycarbonyl, C₁₋₄alkylsulfonyl, C₁₋₄alkylsulfinyl, C₁₋₄haloalkylsulfinyl, formyl, C₁₋₄acyl, haloC₁₋₄acyl, arylC₁₋₄alkoxyC₁₋₄alkyl, C₆₋₁₁aryl substituted by one to three C₁₋₄alkyl groups, C₃₋₆heterocyclyl, C₃₋₁₀heteroaryl, C₃₋₁₀heteroaryl substituted by one to three C₁₋₄alkyl groups, a group —NR₉R₁₀, —NR₉C(O)R₁₀, —NHC(O)—C₁₋₂alkyl, —CR_{9′}═NOR_{10′}, —(CH₂)ᵣN(R_{9′})C(O)R_{10′}, —(CH₂)ᵣNR₉R₁₀ and —O(CH₂)_{q}NR_{9′}R_{10′}, wherein
each R₉ and R₁₀ is independently halo C₁₋₄alkyl, C₁₋₄alkyl, C₅₋₁₀aryl, or where appropriate R₉R₁₀ forms part of a C₃₋₆azacyloalkane ring
each R_{9′} and R_{10′} is independently selected from R₉ and R₁₀ and hydrogen;
each R_{9″} and R_{10″} is independently selected from R_{9′} and R_{10′}, C₁₋₄acyl, haloC₁₋₄acyl, haloaryl and C₁₋₄alkanoyl;
q is selected from 2, 3 or 4;
r is selected from 1, 2, 3 or 4.

In particular, R₁₅ may be C₁₋₄alkoxyC₁₋₄alkyl, haloC₁₋₄alkoxyC₁₋₄alkyl, C₁₋₄alkoxyhaloC₁₋₄alkyl, C₁₋₄alkanoyl, haloC₁₋₄alkanoyl, C₁₋₄alkoxyC₁₋₄alkoxyC₁₋₄alkyl, haloC₁₋₄alkoxyC₁₋₄alkoxyC₁₋₄alkyl, C₁₋₄alkoxyC₁₋₄haloalkoxyC₁₋₄alkyl, C₁₋₄alkoxyC₁₋₄alkoxyC₁₋₄haloalkyl, haloC₁₋₄alkyl (for example trifluoromethyl) or NR_{9′}R_{10′} wherein R_{9′} is C₁₋₄alkyl or haloC₁₋₄alkyl and R_{10′} is C₁₋₄ acyl, haloC₁₋₄ acyl or haloC₅₋₁₁aroyl. There may, for example, be one or two halo substituents; they may be independently selected from, for example, F or Cl.

In a further embodiment, R₁₅ may be C₁₋₄alkoxyC₁₋₄alkyl, haloC₁₋₄alkoxyC₁₋₄alkyl, hydroxyC₁₋₄alkyl, diC₁₋₄alkylaminoC₁₋₄alkoxy, aminoC₁₋₄alkoxy, C₁₋₄alkoxyhaloC₁₋₄alkyl, haloC₁₋₄alkanoyl, C₁₋₄alkoxyC₁₋₄alkoxyC₁₋₄alkyl, haloC₁₋₄alkoxyC₁₋₄alkoxyC₁₋₄alkyl, C₁₋₄alkoxyC₁₋₄haloalkoxyC₁₋₄alkyl, C₁₋₄alkoxyC₁₋₄alkoxyC₁₋₄haloalkyl, haloC₁₋₄alkyl (for example trifluoromethyl), C₁₋₄alkylthio or NR_{9′}R_{10′} wherein R_{9′} is C₁₋₄alkyl or haloC₁₋₄alkyl and R₁₀ is C₁₋₄ acyl, haloC₁₋₄ acyl or haloC₅₋₁₁aroyl

In a further embodiment, at least one of R₁₃, R₁₄, and R₁₅ is selected from C₁₋₄alkoxyC₁₋₄alkyl, haloC₁₋₄alkoxyC₁₋₄alkyl, hydroxyC₁₋₄alkyl, diC₁₋₄alkylaminoC₁₋₄alkoxy aminoC₁₋₄alkoxy, hydroxyC₁₋₄alkyl and C₁₋₄alkylthio.

In a further embodiment Z is a monocyclic or bicyclic heteroaryl group, a bicyclic C₈₋₁₁ aryl group, which heteroaryl or aryl group is optionally substituted as set out above. For example, Z may be a bicyclic C₈₋₁₁aryl group or an optionally substituted pyrrolopyridine and more preferably an optionally substituted 1H-pyrrolo[2,3-b]pyridine. Preferred optional substituents include halogen, hydroxy, oxo, C₁₋₆alkyl, C₁₋₄alkoxy, haloC₁₋₄alkyl, C₁₋₄alkoxyC₁₋₄alkyl, C₁₋₄alkylsulfonyl, C₆₋₁₁arylsulfonyl, C₄₋₉heteroarylsulfonyl, and C₁₋₄acyl.

For example, such a Z group may be substituted with an arylsulfonyl or a heteroarylsulfonyl group, for example with a heteroarylsulfonyl group.

Where there is more than one substituent on any group, the substituents may be different or the same. If substituent(s) is/are present, preferably the number of substituent(s) is 1, 2, 3 or 4.

Specific examples of compounds of the present invention include compounds of Examples 1, 2, 4, 6, 7, 8, 10, 12 to 48 or 50 to 131 as set out below, and salts and solvates thereof:

The compounds of formula (I) may have the ability to crystallise in more than one form. This is a characteristic known as polymorphism, and it is understood that such polymorphic forms (“polymorphs”) are within the scope of formula (I). Polymorphism generally can occur as a response to changes in temperature or pressure or both and can also result from variations in the crystallisation process. Polymorphs can be distinguished by various physical characteristics known in the art such as x-ray diffraction patterns, solubility, and melting point.

Certain of the compounds described herein may exist in stereoisomeric forms (i.e. they may contain one or more asymmetric carbon atoms or may exhibit cis-trans isomerism). The individual stereoisomers (enantiomers and diastereoisomers) and mixtures of these are included within the scope of the present invention. Likewise, it is understood that compounds of formula (I) may exist in tautomeric forms other than that shown in the formula and these are also included within the scope of the present invention.

As referred to above, individual enantiomers of compounds of formula (I) may be prepared and an indication of the preferred stereochemistry for such enantiomers has been given. In a preferred embodiment, an optically pure enantiomer is desired. The term “optically pure enantiomer” means that the compound contains greater than about 90% of the desired isomer by weight, preferably greater than about 95% of the desired isomer by weight, and most preferably greater than about 99% of the desired isomer by weight, said weight percent based upon the total weight of the isomer(s) of the compound.

The compounds of this invention may be made by a variety of methods, including standard chemistry. Any previously defined variable will continue to have the previously defined meaning unless otherwise indicated. Illustrative general synthetic methods are set out below and then specific compounds of the invention are prepared in the working Examples.

Compounds of general formula (I) may be prepared by methods disclosed in the documents hereinbefore referred to and by methods known in the art of organic synthesis as set forth in part by the following synthesis schemes. It is also recognised that in all of the schemes described below, it is well understood that protecting groups for sensitive or reactive groups are employed where necessary in accordance with general principles of chemistry. Protecting groups are manipulated according to standard methods of organic synthesis (T. W. Greene and P. G. M. Wuts (1991) Protecting Groups in Organic Synthesis, John Wiley & Sons). These groups are removed at a convenient stage of the compound synthesis using methods that are readily apparent to those skilled in the art. The selection of processes as well as the reaction conditions and order of their execution shall be consistent with the preparation of compounds of formula (I). Those skilled in the art will recognise if a stereocentre exists in compounds of formula (I). Accordingly, the present invention includes both possible stereoisomers and includes not only racemic compounds but the individual enantiomers as well. Where the stereochemistry is indicated as being variable at certain positions, a mixture of stereoisomers may be obtained, this mixture having been separated where indicated. Stereoisomers may be separated by high-performance liquid chromatography or other appropriate means. When a compound is desired as a single enantiomer, it may be obtained by stereospecific synthesis or by resolution of the final product or any convenient intermediate. Resolution of the final product, an intermediate, or a starting material may be effected by any suitable method known in the art. See, for example, Stereochemistry of Organic Compounds by E. L. Eliel, S. H. Wilen, and L. N. Mander (Wiley-Interscience, 1994).

Typical reaction routes for the preparation of a compound of formula (I) as hereinbefore defined, are shown in the following schemes. It should be noted that, while the schemes illustrate cases wherein Y is —SO₂Me and n is zero, the schemes are applicable for other cases wherein n and Y (including R₂ and m) are as defined for formula (I) above mutatis mutandis, according to methods known to the skilled person. Similarly, the schemes illustrate cases where the leaving group is chlorine, but the leaving group may be any other suitable group. Schemes 5, 6 illustrate methodology for preparing compounds in which Z is phenyl substituted with an alkoxyalkyl group. Scheme 7 illustrates methodology for preparing compounds in which Z is phenyl substituted with an alkoxy group. The methodology is also suitable for the preparation of other molecules of the invention that comprise alkoxyalkyl groups. Starting materials and reagents are known to the skilled person in the art and/or can be prepared using methods known in the art.

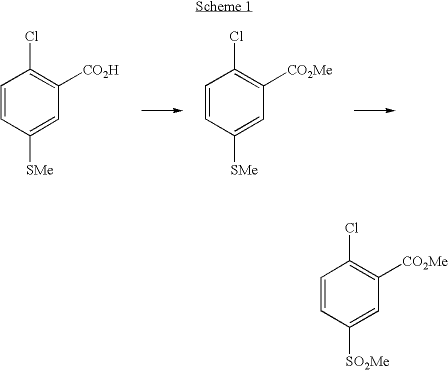

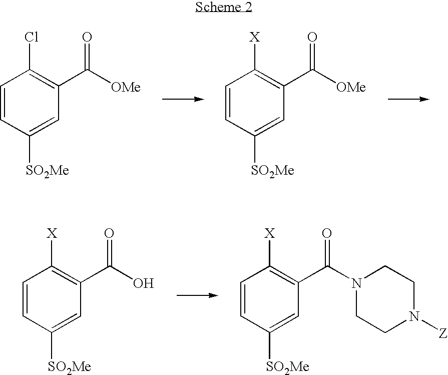

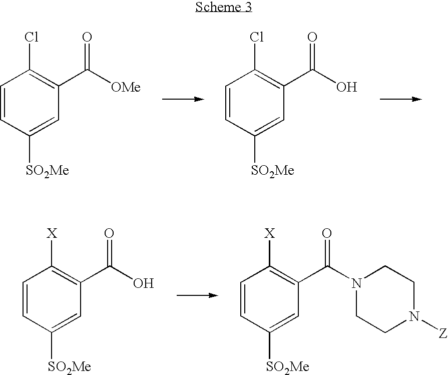

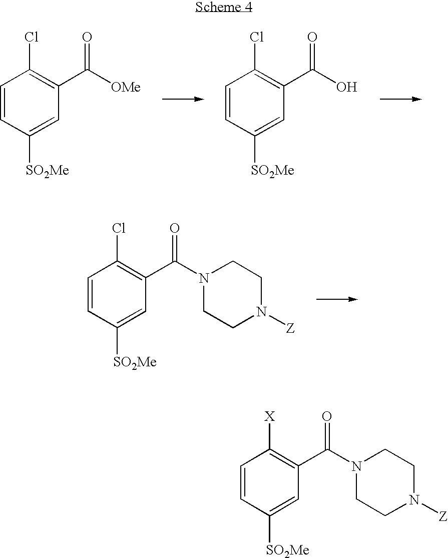

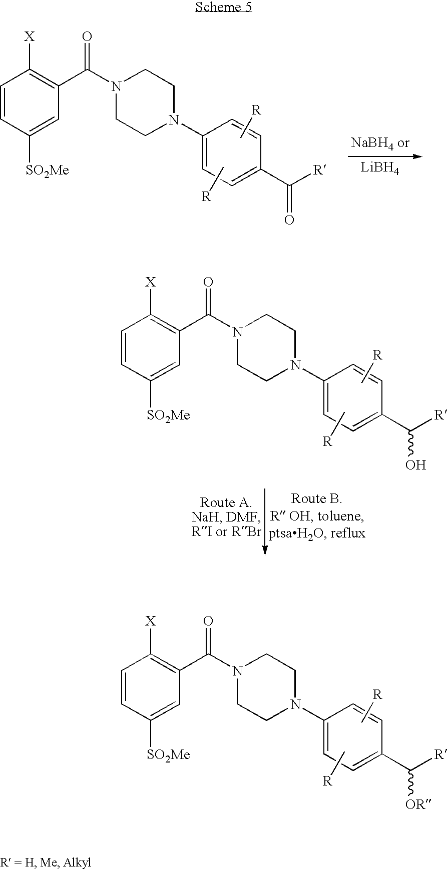

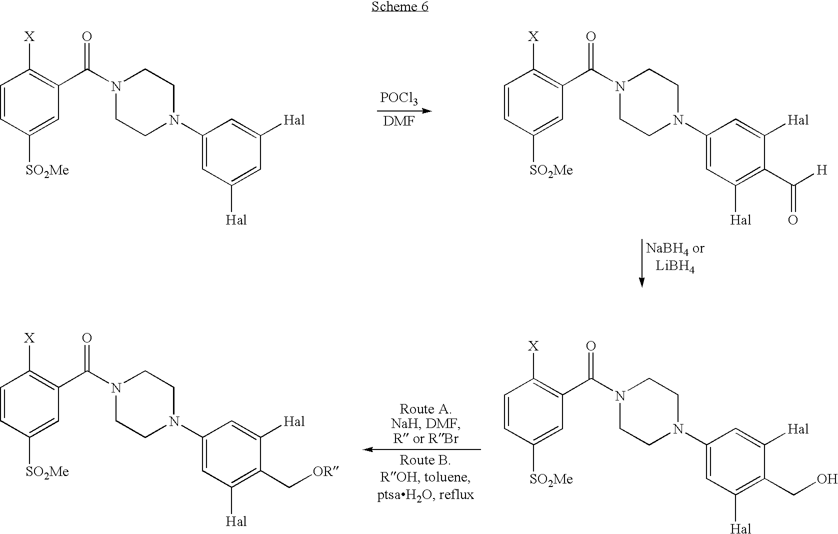

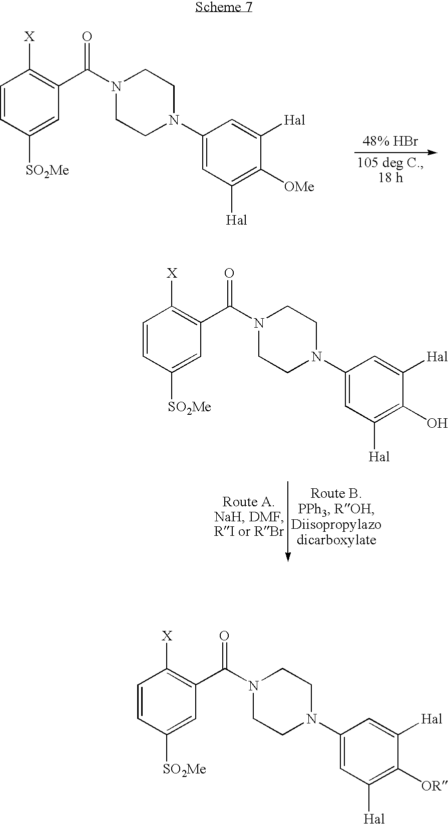

Accordingly, in a second aspect, the present invention provides a method of preparing a compound of formula (I), comprising the step of:

(a) reacting a compound of formula (II):

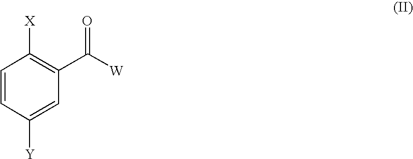

wherein X and Y are as defined for formula (I) and W is —OH or —Cl, with a compound of formula (III):

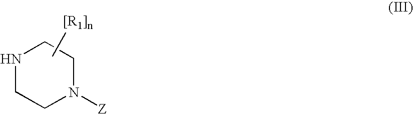

wherein Z, n and R₁ are as defined for formula (I); or

(b) reacting a compound of formula (IV):

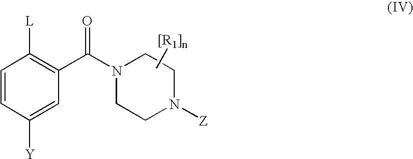

wherein Y, R₁, n and Z are as defined for formula (I) and L is a leaving group such as chloride, bromide or trifluoromethanesulfonate, with a compound of formula (V):

XB(OH)₂ (V)

wherein X is as defined for formula (I);
and thereafter optionally for step (a) or step (b):
removing any protecting groups and/or
converting a compound of formula (I) into another compound of formula (I) and/or
forming a salt or solvate.

The reaction conditions for step (a) are known to the skilled person. The reaction conditions for step (b) are known to the skilled person, for example as set out in N. Miyaura, T. Yanagi and A. Suzuki, Synth. Commun., 1981, 11, 513; N. Miyaura, and A. Suzuki, Chem. Rev., 1995, 95, 2457.

Compounds of formulae (II)-(V) are commercially available, or may be made according to known methods available to the skilled person, or may be made according to methods disclosed herein.

Compounds of formula (I) can be converted into further compounds of formula (I) using standard techniques. For example, and by way of illustration rather than limitation, possible conversion reactions include acylation with an appropriate acylating agent such as acetyl chloride, alkylation using an appropriate alkylating reagent such as methyl iodide, and sulfonylation using a sulfonylating agent such as methanesulfonic anhydride.

Pharmaceutically acceptable salts may be prepared conventionally by reaction with the appropriate acid or acid derivative by procedures known to those skilled in the art.

The compounds of the present invention inhibit the GlyT1 transporter. The compounds may selectively inhibit the GlyT1 transporter over the GlyT2 transporter.

Such compounds would be suitable for the treatment of certain neurological and neuropsychiatric disorders. As used herein, the terms “treatment” and “treating” refer to the alleviation and/or cure of established symptoms as well as prophylaxis.

The affinities of the compounds of this invention for the GlyT1 transporter can be determined by the following assay:

HEK293 cells expressing the Glycine (Type 1) transporter were grown in cell medium (DMEM/NUT mix F12) containing 2 mM L-Glutamine, 0.8 mg/mL G418 and 10% heat inactivated fetal calf serum (Gibco BRL) at 37° C. in 5% CO₂. Cells grown to 70-80% confluency in T175 flasks were harvested and resuspended at 1.6×10⁶ cells/ml in assay buffer [NaCl (140 mM), KCl (5.4 mM), CaCl₂ (1.8 mM), MgSO₄ (0.8 mM), HEPES (20 mM), glucose (5 mM) and alanine (5 mM), pH 7.4]. An equal volume of Leadseeker™ SPA beads (12.5 mg/ml suspended in assay buffer) was added to the cells and 25 mL of the cell/bead suspension transferred to each well of a 384-well white solid bottom plate (20,000 cells/well) that contained 14 mL of assay buffer. Compounds were prepared as 10 mM stocks in DMSO. Two-fold serial dilutions of the compounds were made in DMSO from a top concentration of 5 mM. 1 mL of compound at each concentration was added to the assay plate using 384-well parallel dispensing. Substrate (10 mL) was added to each well [1:40 dilution of [³H]-glycine in assay buffer containing 5 mM glycine). Final DMSO concentration=2%. Data was collected using a PerkinElmer Viewlux as 5 minute exposures. IC₅₀ values were determined using Grafit.

The following alternative assay may also be used:

HEK293 cells expressing the Glycine (Type 1) transporter were grown in cell medium (DMEM/NUT mix F12) containing 2 mM L-Glutamine, 0.8 mg/mL G418 and 10% heat inactivated fetal calf serum (Gibco BRL) at 37° C. in 5% CO₂. Cells grown to 70-80% confluency in T175 flasks were harvested and resuspended at 4×10⁵ cells/ml in assay buffer [NaCl (140 mM), KCl (5.4 mM), CaCl₂ (1.8 mM), MgSO₄ (0.8 mM), HEPES (20 mM), glucose (5 mM) and alanine (5 mM), pH 7.4]. An equal volume of Leadseeker SPA beads (12.5 mg/ml suspended in assay buffer) was added to the cell suspension. Compounds were prepared as 10 mM stocks in DMSO. 2.5 fold serial dilutions of the compounds were made in DMSO from a top conc of 2.5 mM. 100 mL of compound at each concentration was added to the assay plate (384-well white solid bottom plate) using the hummingbird dispenser. 5 uL of the cell/bead mix was then added on top of the compound using a multidrop dispenser. Substrate (5 uL) was then added to each well (1:100 dilution of H3-glycine in assay buffer containing 2.5 uM glycine) Data was collected using a PerkinElmer Viewlux as 5 minute exposures. plC50 data values were determined using Activity Base.

Compounds are considered to have activity at the GlyT1 transporter if they have a plC₅₀ if 5.0 or above. The example compounds below were found to have a plC₅₀ at the GlyT1 transporter of greater than 5.0. Preferred compounds of the invention were found to have a plC₅₀ at the GlyT1 transporter of greater than 6.0.

Compounds for use according to the invention may be administered as the raw material but the active ingredients are preferably provided in the form of pharmaceutical compositions.

Accordingly, in a further aspect of the invention, there is provided a pharmaceutical composition comprising a compound of formula (I) as hereinbefore described or a salt or solvate thereof, and at least one pharmaceutically acceptable carrier, diluent or excipient. These pharmaceutical compositions may be used in the treatment of clinical conditions for which a GlyT1 inhibitor is indicated such as, for example, schizophrenia. The carrier must be pharmaceutically acceptable to the recipient and must be compatible with, i.e. not have a deleterious effect upon, the other ingredients in the composition. The carrier may be a solid or a liquid and is preferably formulated with at least one compound of formula (I) or a salt or solvate thereof as a unit dose formulation. If desired, other physiologically active ingredients may also be incorporated in the pharmaceutical compositions of the invention.

These pharmaceutical compositions may be used in the treatment of clinical conditions for which a GlyT1 inhibitor is indicated such as, for example, schizophrenia. The carrier must be pharmaceutically acceptable to the recipient and must be compatible with, i.e. not have a deleterious effect upon, the other ingredients in the composition. The carrier may be a solid or a liquid and is preferably formulated with at least one compound of formula (I) or a salt or solvate thereof as a unit dose formulation. If desired, other physiologically active ingredients may also be incorporated in the pharmaceutical compositions of the invention.

Within the context of the present invention, the terms used herein are classified in the Diagnostic and Statistical Manual of Mental Disorders, 4^{th} Edition, published by the American Psychiatric Association (DSM-IV) and/or the International Classification of Diseases, 10^{th} Edition (ICD-10). The various subtypes of the disorders mentioned herein are contemplated as part of the present invention. Numbers in brackets after the listed diseases below refer to the classification code in DSM-IV.

In particular, the compounds of formula (I) are of use in the treatment of schizophrenia including the subtypes Paranoid Type (295.30), Disorganised Type (295.10), Catatonic Type (295.20), Undifferentiated Type (295.90) and Residual Type (295.60); Schizophreniform Disorder (295.40); Schizoaffective Disorder (295.70) including the subtypes Bipolar Type and Depressive Type; Delusional Disorder (297.1) including the subtypes Erotomanic Type, Grandiose Type, Jealous Type, Persecutory Type, Somatic Type, Mixed Type and Unspecified Type; Brief Psychotic Disorder (298.8); Shared Psychotic Disorder (297.3); Psychotic Disorder Due to a General Medical Condition including the subtypes With Delusions and With Hallucinations; Substance-Induced Psychotic Disorder including the subtypes With Delusions (293.81) and With Hallucinations (293.82); and Psychotic Disorder Not Otherwise Specified (298.9).

The compounds of formula (I) are also of use in the treatment of mood disorders including Major Depressive Episode, Manic Episode, Mixed Episode and Hypomanic Episode; Depressive Disorders including Major Depressive Disorder, Dysthymic Disorder (300.4), Depressive Disorder Not Otherwise Specified (311); Bipolar Disorders including Bipolar I Disorder, Bipolar II Disorder (Recurrent Major Depressive Episodes with Hypomanic Episodes) (296.89), Cyclothymic Disorder (301.13) and Bipolar Disorder Not Otherwise Specified (296.80); Other Mood Disorders including Mood Disorder Due to a General Medical Condition (293.83) which includes the subtypes With Depressive Features, With Major Depressive-like Episode, With Manic Features and With Mixed Features), Substance-Induced Mood Disorder (including the subtypes With Depressive Features, With Manic Features and With Mixed Features) and Mood Disorder Not Otherwise Specified (296.90).

The compounds of formula (I) are also of use in the treatment of anxiety disorders including Panic Attack, Agoraphobia, Panic Disorder, Agoraphobia Without History of Panic Disorder (300.22), Specific Phobia (300.29) including the subtypes Animal Type, Natural Environment Type, Blood-Injection-Injury Type, Situational Type and Other Type), Social Phobia (300.23), Obsessive-Compulsive Disorder (300.3), Posttraumatic Stress Disorder (309.81), Acute Stress Disorder (308.3), Generalized Anxiety Disorder (300.02), Anxiety Disorder Due to a General Medical Condition (293.84), Substance-Induced Anxiety Disorder and Anxiety Disorder Not Otherwise Specified (300.00).

The compounds of formula (I) are also of use in the treatment of substance-related disorders including Substance Use Disorders such as Substance Dependence and Substance Abuse; Substance-Induced Disorders such as Substance Intoxication, Substance Withdrawal, Substance-Induced Delirium, Substance-Induced Persisting Dementia, Substance-Induced Persisting Amnestic Disorder, Substance-Induced Psychotic Disorder, Substance-Induced Mood Disorder, Substance-Induced Anxiety Disorder, Substance-Induced Sexual Dysfunction, Substance-Induced Sleep Disorder and Hallucinogen Persisting Perception Disorder (Flashbacks); Alcohol-Related Disorders such as Alcohol Dependence (303.90), Alcohol Abuse (305.00), Alcohol Intoxication (303.00), Alcohol Withdrawal (291.81), Alcohol Intoxication Delirium, Alcohol Withdrawal Delirium, Alcohol-Induced Persisting Dementia, Alcohol-Induced Persisting Amnestic Disorder, Alcohol-Induced Psychotic Disorder, Alcohol-Induced Mood Disorder, Alcohol-Induced Anxiety Disorder, Alcohol-induced Sexual Dysfunction, Alcohol-induced Sleep Disorder and Alcohol-Related Disorder Not Otherwise Specified (291.9); Amphetamine (or Amphetamine-Like)-Related Disorders such as Amphetamine Dependence (304.40), Amphetamine Abuse (305.70), Amphetamine Intoxication (292.89), Amphetamine Withdrawal (292.0), Amphetamine Intoxication Delirium, Amphetamine Induced Psychotic Disorder, Amphetamine-Induced Mood Disorder, Amphetamine-Induced Anxiety Disorder, Amphetamine-Induced Sexual Dysfunction, Amphetamine-Induced Sleep Disorder and Amphetamine-Related Disorder Not Otherwise Specified (292.9); Caffeine Related Disorders such as Caffeine Intoxication (305.90), Caffeine-induced Anxiety Disorder, Caffeine-Induced Sleep Disorder and Caffeine-Related Disorder Not Otherwise Specified (292.9); Cannabis-Related Disorders such as Cannabis Dependence (304.30), Cannabis Abuse (305.20), Cannabis Intoxication (292.89), Cannabis Intoxication Delirium, Cannabis-Induced Psychotic Disorder, Cannabis-Induced Anxiety Disorder and Cannabis-Related Disorder Not Otherwise Specified (292.9); Cocaine-Related Disorders such as Cocaine Dependence (304.20), Cocaine Abuse (305.60), Cocaine Intoxication (292.89), Cocaine Withdrawal (292.0), Cocaine Intoxication Delirium, Cocaine-induced Psychotic Disorder, Cocaine-induced Mood Disorder, Cocaine-Induced Anxiety Disorder, Cocaine-Induced Sexual Dysfunction, Cocaine-Induced Sleep Disorder and Cocaine-Related Disorder Not Otherwise Specified (292.9); Hallucinogen-Related Disorders such as Hallucinogen Dependence (304.50), Hallucinogen Abuse (305.30), Hallucinogen Intoxication (292.89), Hallucinogen Persisting Perception Disorder (Flashbacks) (292.89), Hallucinogen Intoxication Delirium, Hallucinogen-Induced Psychotic Disorder, Hallucinogen-Induced Mood Disorder, Hallucinogen-Induced Anxiety Disorder and Hallucinogen-Related Disorder Not Otherwise Specified (292.9); Inhalant-Related Disorders such as Inhalant Dependence (304.60), Inhalant Abuse (305.90), Inhalant Intoxication (292.89), Inhalant Intoxication Delirium, Inhalant-Induced Persisting Dementia, Inhalant-Induced Psychotic Disorder, Inhalant-Induced Mood Disorder, Inhalant-Induced Anxiety Disorder and Inhalant-Related Disorder Not Otherwise Specified (292.9); Nicotine-Related Disorders such as Nicotine Dependence (305.1), Nicotine Withdrawal (292.0) and Nicotine-Related Disorder Not Otherwise Specified (292.9); Opioid-Related Disorders such as Opioid Dependence (304.00), Opioid Abuse (305.50), Opioid Intoxication (292.89), Opioid Withdrawal (292.0), Opioid Intoxication Delirium, Opioid-Induced Psychotic Disorder, Opioid-Induced Mood Disorder, Opioid-Induced Sexual Dysfunction, Opioid-Induced Sleep Disorder and Opioid-Related Disorder Not Otherwise Specified (292.9); Phencyclidine (or Phencyclidine-Like)-Related Disorders such as Phencyclidine Dependence (304.60), Phencyclidine Abuse (305.90), Phencyclidine Intoxication (292.89), Phencyclidine Intoxication Delirium, Phencyclidine-Induced Psychotic Disorder, Phencyclidine-Induced Mood Disorder, Phencyclidine-Induced Anxiety Disorder and Phencyclidine-Related Disorder Not Otherwise Specified (292.9); Sedative-, Hypnotic-, or Anxiolytic-Related Disorders such as Sedative, Hypnotic, or Anxiolytic Dependence (304.10), Sedative, Hypnotic, or Anxiolytic Abuse (305.40), Sedative, Hypnotic, or Anxiolytic Intoxication (292.89), Sedative, Hypnotic, or Anxiolytic Withdrawal (292.0), Sedative, Hypnotic, or Anxiolytic Intoxication Delirium, Sedative, Hypnotic, or Anxiolytic Withdrawal Delirium, Sedative-, Hypnotic-, or Anxiolytic-Persisting Dementia, Sedative-, Hypnotic-, or Anxiolytic-Persisting Amnestic Disorder, Sedative-, Hypnotic-, or Anxiolytic-Induced Psychotic Disorder, Sedative-, Hypnotic-, or Anxiolytic-Induced Mood Disorder, Sedative-, Hypnotic-, or Anxiolytic-Induced Anxiety Disorder Sedative-, Hypnotic-, or Anxiolytic-Induced Sexual Dysfunction, Sedative-, Hypnotic-, or Anxiolytic-Induced Sleep Disorder and Sedative-, Hypnotic-, or Anxiolytic-Related Disorder Not Otherwise Specified (292.9); Polysubstance-Related Disorder such as Polysubstance Dependence (304.80); and Other (or Unknown) Substance-Related Disorders such as Anabolic Steroids, Nitrate Inhalants and Nitrous Oxide.

The compounds of formula (I) are also of use in the treatment of sleep disorders including primary sleep disorders such as Dyssomnias such as Primary Insomnia (307.42), Primary Hypersomnia (307.44), Narcolepsy (347), Breathing-Related Sleep Disorders (780.59), Circadian Rhythm Sleep Disorder (307.45) and Dyssomnia Not Otherwise Specified (307.47); primary sleep disorders such as Parasomnias such as Nightmare Disorder (307.47), Sleep Terror Disorder (307.46), Sleepwalking Disorder (307.46) and Parasomnia Not Otherwise Specified (307.47); Sleep Disorders Related to Another Mental Disorder such as Insomnia Related to Another Mental Disorder (307.42) and Hypersomnia Related to Another Mental Disorder (307.44); Sleep Disorder Due to a General Medical Condition; and Substance-Induced Sleep Disorder including the subtypes Insomnia Type, Hypersomnia Type, Parasomnia Type and Mixed Type.

The compounds of formula (I) are also of use in the treatment of eating disorders such as Anorexia Nervosa (307.1) including the subtypes Restricting Type and Binge-Eating/Purging Type; Bulimia Nervosa (307.51) including the subtypes Purging Type and Nonpurging Type; Obesity; Compulsive Eating Disorder; and Eating Disorder Not Otherwise Specified (307.50).

The compounds of formula (I) are also of use in the treatment of Autistic Disorder (299.00); Attention-Deficit/Hyperactivity Disorder including the subtypes Attention-Deficit/Hyperactivity Disorder Combined Type (314.01), Attention-Deficit/Hyperactivity Disorder Predominantly Inattentive Type (314.00), Attention-Deficit/Hyperactivity Disorder Hyperactive-impulse Type (314.01) and Attention-Deficit/Hyperactivity Disorder Not Otherwise Specified (314.9); Hyperkinetic Disorder; Disruptive Behaviour Disorders such as Conduct Disorder including the subtypes childhood-onset type (321.81), Adolescent-Onset Type (312.82) and Unspecified Onset (312.89), Oppositional Defiant Disorder (313.81) and Disruptive Behaviour Disorder Not Otherwise Specified; and Tic Disorders such as Tourette's Disorder (307.23).

The compounds of formula (I) are also of use in the treatment of Personality Disorders including the subtypes Paranoid Personality Disorder (301.0), Schizoid Personality Disorder (301.20), Schizotypal Personality Disorder (301,22), Antisocial Personality Disorder (301.7), Borderline Personality Disorder (301,83), Histrionic Personality Disorder (301.50), Narcissistic Personality Disorder (301,81), Avoidant Personality Disorder (301.82), Dependent Personality Disorder (301.6), Obsessive-Compulsive Personality Disorder (301.4) and Personality Disorder Not Otherwise Specified (301.9).

The compounds of Formula (I) are also of use in the enhancement of cognition including the treatment of cognition impairment in other diseases such as schizophrenia, bipolar disorder, depression, other psychiatric disorders and psychotic conditions associated with cognitive impairment. Within the context of the present invention, the term cognitive impairment includes for example the treatment of impairment of cognitive functions including attention, orientation, learning disorders, memory (i.e. memory disorders, amnesia, amnesic disorders, transient global amnesia syndrome and age-associated memory impairment) and language function; cognitive impairment as a result of stroke, Alzheimer's disease, Huntington's disease, Pick disease, Aids-related dementia or other dementia states such as Multiinfarct dementia, alcoholic dementia, hypothyroidism-related dementia, and dementia associated to other degenerative disorders such as cerebellar atrophy and amyotropic lateral sclerosis; other acute or sub-acute conditions that may cause cognitive decline such as delirium or depression (pseudodementia states) trauma, head trauma, age related cognitive decline, stroke, neurodegeneration, drug-induced states, neurotoxic agents, mild cognitive impairment, age related cognitive impairment, autism related cognitive impairment, Down's syndrome, cognitive deficit related to psychosis, and post-electroconvulsive treatment related cognitive disorders; and dyskinetic disorders such as Parkinson's disease, neuroleptic-induced parkinsonism, and tardive dyskinesias.

The compounds of formula (I) are also of use in the treatment of sexual dysfunctions including Sexual Desire Disorders such as Hypoactive Sexual Desire Disorder (302.71), and Sexual Aversion Disorder (302.79); sexual arousal disorders such as Female Sexual Arousal Disorder (302.72) and Male Erectile Disorder (302.72); orgasmic disorders such as Female Orgasmic Disorder (302.73), Male Orgasmic Disorder (302.74) and Premature Ejaculation (302.75); sexual pain disorder such as Dyspareunia (302.76) and Vaginismus (306.51); Sexual Dysfunction Not Otherwise Specified (302.70); paraphilias such as Exhibitionism (302.4), Fetishism (302.81), Frotteurism (302.89), Pedophilia (302.2), Sexual Masochism (302.83), Sexual Sadism (302.84), Transvestic Fetishism (302.3), Voyeurism (302.82) and Paraphilia Not Otherwise Specified (302.9); gender identity disorders such as Gender Identity Disorder in Children (302.6) and Gender Identity Disorder in Adolescents or Adults (302.85); and Sexual Disorder Not Otherwise Specified (302.9).

The invention also provides a compound of formula (I) as hereinbefore described or a pharmaceutically acceptable salt or solvate thereof for use in the treatment of schizophrenia, mood disorders, anxiety disorders, substance-related disorders, sleep disorders, eating disorders, autistic disorder, attention-deficit/hyperactivity disorder, disruptive behaviour disorder, tic disorders, personality disorders, cognition impairment in other diseases, sexual dysfunction, Parkinson's disease, dyskinetic disorders, depression, bipolar disorder, cognitive impairment, obesity, emesis, movement disorders, obsessive-compulsive disorders, amnesia, aggression, vertigo, dementia and circadian rhythm disorders.

The invention also provides a compound of formula (I) as hereinbefore described or a pharmaceutically acceptable salt or solvate thereof for use in the treatment of psychotic disorders, schizophrenia, Parkinson's disease, substance abuse, dyskinetic disorders, depression, bipolar disorder, anxiety, cognitive impairment, eating disorders, obesity, sexual dysfunction, sleep disorders, emesis, movement disorders, obsessive-compulsive disorders, amnesia, aggression, autism, vertigo, dementia, circadian rhythm disorders and gastric motility disorders.

In another aspect of the invention, there is provided a method of treating a mammal, including a human, suffering from or susceptible to a disorder mediated by GlyT1, which comprises administering an effective amount of a compound of formula (I) as hereinbefore defined or a salt or solvate thereof.

The invention also provides a method of treating schizophrenia, mood disorders, anxiety disorders, substance-related disorders, sleep disorders, eating disorders, autistic disorder, attention-deficit/hyperactivity disorder, disruptive behaviour disorder, tic disorders, personality disorders, cognition impairment in other diseases, sexual dysfunction, Parkinson's disease, dyskinetic disorders, depression, bipolar disorder, cognitive impairment, obesity, emesis, movement disorders, obsessive-compulsive disorders, amnesia, aggression, vertigo, dementia and circadian rhythm disorders which comprises administering to a mammal in need thereof an effective amount of a compound of formula (I) as hereinbefore described or a pharmaceutically acceptable salt or solvate thereof.

The invention also provides a method of treating psychotic disorders, schizophrenia, Parkinson's disease, substance abuse, dyskinetic disorders, depression, bipolar disorder, anxiety, cognitive impairment, eating disorders, obesity, sexual dysfunction, sleep disorders, emesis, movement disorders, obsessive-compulsive disorders, amnesia, aggression, autism, vertigo, dementia, circadian rhythm disorders and gastric motility disorders which comprises administering to a mammal in need thereof an effective amount of a compound of formula (I) as hereinbefore described or a pharmaceutically acceptable salt or solvate thereof.

In another aspect of the invention, there is provided use of a compound of formula (I) as hereinbefore defined or a salt or solvate thereof in the preparation of a medicament for the treatment of a disorder mediated by GlyT1.

Preferably, the disorder mediated by GlyT1 to be treated by the use or method as hereinbefore described is a psychosis, including schizophrenia, dementia and attention deficit disorders, particularly schizophrenia.

The invention also provides the use of a compound of formula (I) as hereinbefore described or a pharmaceutically acceptable salt or solvate thereof in the manufacture of a medicament for the treatment of schizophrenia, mood disorders, anxiety disorders, substance-related disorders, sleep disorders, eating disorders, autistic disorder, attention-deficit/hyperactivity disorder, disruptive behaviour disorder, tic disorders, personality disorders, cognition impairment in other diseases, sexual dysfunction, Parkinson's disease, dyskinetic disorders, depression, bipolar disorder, cognitive impairment, obesity, emesis, movement disorders, obsessive-compulsive disorders, amnesia, aggression, vertigo, dementia and circadian rhythm disorders.

The invention also provides the use of a compound of formula (I) as hereinbefore described or a pharmaceutically acceptable salt or solvate thereof in the manufacture of a medicament for the treatment of psychotic disorders, schizophrenia, Parkinson's disease, substance abuse, dyskinetic disorders, depression, bipolar disorder, anxiety, cognitive impairment, eating disorders, obesity, sexual dysfunction, sleep disorders, emesis, movement disorders, obsessive-compulsive disorders, amnesia, aggression, autism, vertigo, dementia, circadian rhythm disorders and gastric motility disorders.

As used herein, the term “effective amount” means that amount of a drug or pharmaceutical agent that will elicit the biological or medical response of a tissue, system, animal or human that is being sought, for instance, by a researcher or clinician.

Compounds for use according to the invention may be administered as the raw material but the active ingredients are preferably provided in the form of pharmaceutical compositions.

Accordingly, in a further aspect of the invention, there is provided a pharmaceutical composition comprising a compound of formula (I) as hereinbefore described or a salt or solvate thereof, and at least one pharmaceutically acceptable carrier, diluent or excipient.

These pharmaceutical compositions may be used in the treatment of clinical conditions for which a GlyT1 inhibitor is indicated such as, for example, schizophrenia. The carrier must be pharmaceutically acceptable to the recipient and must be compatible with, i.e. not have a deleterious effect upon, the other ingredients in the composition. The carrier may be a solid or a liquid and is preferably formulated with at least one compound of formula (I) or a salt or solvate thereof as a unit dose formulation. If desired, other physiologically active ingredients may also be incorporated in the pharmaceutical compositions of the invention.

It will be appreciated by those skilled in the art that the compounds according to the invention may advantageously be used in conjunction with one or more other therapeutic agents, for instance, different antidepressant agents such as 5HT3 antagonists, serotonin agonists, NK-1 antagonists, selective serotonin reuptake inhibitors (SSRI), noradrenaline re-uptake inhibitors (SNRI), tricyclic antidepressants, dopaminergic antidepressants, H3 antagonists, 5HT1A antagonists, 5HT1B antagonists, 5HT1D antagonists, D1 agonists, M1 agonists and/or anticonvulsant agents, as well as atypical antipsychotic drugs and cognitive enhancers.

Suitable 5HT3 antagonists which may be used in combination of the compounds of the inventions include for example ondansetron, granisetron, metoclopramide.

Suitable serotonin agonists which may be used in combination with the compounds of the invention include sumatriptan, rauwolscine, yohimbine, metoclopramide.

Suitable SSRIs which may be used in combination with the compounds of the invention include fluoxetine, citalopram, femoxetine, fluvoxamine, paroxetine, indalpine, sertraline, zimeldine.

Suitable SNRIs which may be used in combination with the compounds of the invention include venlafaxine and reboxetine.

Suitable tricyclic antidepressants which may be used in combination with a compound of the invention include imipramine, amitriptyline, clomipramine and nortriptyline.

Suitable dopaminergic antidepressants which may be used in combination with a compound of the invention include bupropion and amineptine.

Suitable anticonvulsant agents which may be used in combination of the compounds of the invention include for example divalproex, carbamazepine and diazepam.

Suitable atypical antipsychotic drugs which which may be used in combination of the compounds of the invention include for example risperidone, olanzapine, ziprasidone, aripiprazole and clozapine.

It will be appreciated that the compounds of the combination or composition may be administered simultaneously (either in the same or different pharmaceutical formulations), separately or sequentially.

The compounds of formula (I) and their pharmaceutically acceptable salts and solvates thereof are also suitable for combination with other typical and atypical antipsychotics to provide improved treatment of psychotic disorders. Particular advantages associated with the combinations, uses and methods of treatment of compounds of formula (I) and their pharmaceutically acceptable salts and solvates thereof include equivalent or improved efficacy at doses of administration which are lower than those commonly used for the individual components. Improved treatments of positive symptoms and/or negative symptoms and/or cognitive symptoms of the psychotic disorder may also be observed. The combinations, uses and methods of treatment of the invention may also provide advantages in treatment of patients who fail to respond adequately or who are resistant to treatment with certain neuroleptic agents.

The combination therapies of the invention are preferably administered adjunctively. By adjunctive administration is meant the coterminous or overlapping administration of each of the components in the form of separate pharmaceutical compositions or devices. This regime of therapeutic administration of two or more therapeutic agents is referred to generally by those skilled in the art and herein as adjunctive therapeutic administration; it is also known as add-on therapeutic administration. Any and all treatment regimes in which a patient receives separate but coterminous or overlapping therapeutic administration of the compounds of formula (I) or a pharmaceutically acceptable salt or solvate thereof and at least one neuroleptic agent are within the scope of the current invention. In one embodiment of adjunctive therapeutic administration as described herein, a patient is typically stabilised on a therapeutic administration of one or more of the of the components for a period of time and then receives administration of another component. Within the scope of this invention, it is preferred that the compounds of formula (I) or a pharmaceutically acceptable salt or solvate thereof is administered as adjunctive therapeutic treatment to patients who are receiving administration of at least one neuroleptic agent, but the scope of the invention also includes the adjunctive therapeutic administration of at least one neuroleptic agent to patients who are receiving administration of compounds of formula (I) or a pharmaceutically acceptable salt or solvate thereof.

The combination therapies of the invention may also be administered simultaneously. By simultaneous administration is meant a treatment regime wherein the individual components are administered together, either in the form of a single pharmaceutical composition or device comprising or containing both components, or as separate compositions or devices, each comprising one of the components, administered simultaneously. Such combinations of the separate individual components for simultaneous combination may be provided in the form of a kit-of-parts.

In a further aspect therefore, the invention provides a method of treatment of a psychotic disorder by adjunctive therapeutic administration of compounds of formula (I) or a pharmaceutically acceptable salt or solvate thereof to a patient receiving therapeutic administration of at least one neuroleptic agent. In a further aspect, the invention provides the use of compounds of formula (I) or a pharmaceutically acceptable salt or solvate thereof in the manufacture of a medicament for adjunctive therapeutic administration for the treatment of a psychotic disorder in a patient receiving therapeutic administration of at least one neuroleptic agent. The invention further provides compounds of formula (I) or a pharmaceutically acceptable salt or solvate thereof for use for adjunctive therapeutic administration for the treatment of a psychotic disorder in a patient receiving therapeutic administration of at least one neuroleptic agent.

In a further aspect, the invention provides a method of treatment of a psychotic disorder by adjunctive therapeutic administration of at least one neuroleptic agent to a patient receiving therapeutic administration of compounds of formula (I) or a pharmaceutically acceptable salt or solvate thereof. In a further aspect, the invention provides the use of at least one neuroleptic agent in the manufacture of a medicament for adjunctive therapeutic administration for the treatment of a psychotic disorder in a patient receiving therapeutic administration of compounds of formula (I) or a pharmaceutically acceptable salt or solvate thereof. The invention further provides at least one neuroleptic agent for adjunctive therapeutic administration for the treatment of a psychotic disorder in a patient receiving therapeutic administration of compounds of formula (I) or a pharmaceutically acceptable salt or solvate thereof.

In a further aspect, the invention provides a method of treatment of a psychotic disorder by simultaneous therapeutic administration of compounds of formula (I) or a pharmaceutically acceptable salt or solvate thereof in combination with at least one neuroleptic agent. The invention further provides the use of a combination of compounds of formula (I) or a pharmaceutically acceptable salt or solvate thereof and at least one neuroleptic agent in the manufacture of a medicament for simultaneous therapeutic administration in the treatment of a psychotic disorder. The invention further provides the use of compounds of formula (I) or a pharmaceutically acceptable salt thereof in the manufacture of a medicament for simultaneous therapeutic administration with at least one neuroleptic agent in the treatment of a psychotic disorder. The invention further provides compounds of formula (I) or a pharmaceutically acceptable salt thereof for use for simultaneous therapeutic administration with at least one neuroleptic agent in the treatment of a psychotic disorder. The invention further provides the use of at least one neuroleptic agent in the manufacture of a medicament for simultaneous therapeutic administration with compounds of formula (I) or a pharmaceutically acceptable salt thereof in the treatment of a psychotic disorder.

In further aspects, the invention provides a method of treatment of a psychotic disorder by simultaneous therapeutic administration of a pharmaceutical composition comprising compounds of formula (I) or a pharmaceutically acceptable salt or solvate thereof and at least one mood stabilising or antimanic agent, a pharmaceutical composition comprising compounds of formula (I) or a pharmaceutically acceptable salt or solvate thereof and at least one mood stabilising or antimanic agent, the use of a pharmaceutical composition comprising compounds of formula (I) or a pharmaceutically acceptable salt or solvate thereof and at least one mood stabilising or antimanic agent in the manufacture of a medicament for the treatment of a psychotic disorder, and a pharmaceutical composition comprising compounds of formula (I) or a pharmaceutically acceptable salt or solvate thereof and at least one mood stabilising or antimanic agent for use in the treatment of a psychotic disorder.

In a further aspect, the invention provides a kit-of-parts for use in the treatment of a psychotic disorder comprising a first dosage form comprising compounds of formula (I) or a pharmaceutically acceptable salt or solvate thereof and one or more further dosage forms each comprising a neuroleptic agent for simultaneous therapeutic administration.

Within the context of the present invention, the term psychotic disorder includes those disorders mentioned above, such as schizophrenia, mood disorders, anxiety disorders, substance-related disorders, sleep disorders, eating disorders, autistic disorder, attention-deficit/hyperactivity disorder, disruptive behaviour disorder, tic disorders, personality disorders, cognition impairment in other diseases, sexual dysfunction, dyskinetic disorders, depression, bipolar disorder, cognitive impairment and obsessive-compulsive disorders and all the various forms of the disorders as mentioned herein which are contemplated as part of the present invention.

Examples of neuroleptic/antipsychotic drugs that are useful in the present invention include, but are not limited to: butyrophenones, such as haloperidol, pimozide, and droperidol; phenothiazines, such as chlorpromazine, thioridazine, mesoridazine, trifluoperazine, perphenazine, fluphenazine, thiflupromazine, prochlorperazine, and acetophenazine; thioxanthenes, such as thiothixene and chlorprothixene thienobenzodiazepines; dibenzodiazepines; benzisoxazoles; dibenzothiazepines; imidazolidinones; benzisothiazolyl-piperazines; triazine such as lamotrigine; dibenzoxazepines, such as loxapine; dihydroindolones, such as molindone; aripiprazole; and derivatives thereof that have antipsychotic activity.

Examples of neuroleptic drugs that are preferred for use in the present invention are shown in Table 1.

TABLE 1
Neuroleptic drugs

| | | | | Dosage |
|---|---|---|---|---|
| Common | | Route of | | Range and |
| Name | Trade Name | Administration | Form | (Median)^{a} |
| Clozapine | CLOZARIL | oral | tablets | 12.5-900 mg/day |
| | | | | (300-900 mg/day) |
| Olanzapine | ZYPREXA | oral | tablets | 5-25 mg/day |
| | | | | (10-25 mg/day) |
| Ziprasidone | GEODON | oral | capsules | 20-80 mg/twice a |
| | | | | day |
| | | | | (80-160 mg/day) |
| Risperidone | RISPERDAL | oral | solution tablets | 2-16 mg/day |
| | | | | tablets |
| | | | | (4-12 mg/day) |
| Quetiapine | SEROQUEL | oral | tablets | 50-900 mg/day |
| fumarate | | | | (300-900 mg/day) |
| Sertindole | SERLECT | | | (4-24 mg/day) |
| Amisulpride | | | | |
| Haloperidol | HALDOL | oral | tablets | 1-100 mg/day |
| | | | | (1-15 mg/day) |
| Haloperidol | HALDOL | parenteral | injection | |
| Decanoate | Decanoate | | | |
| Haloperidol lactate | HALDOL | oral | solution | |
| | INTENSOL | parenteral | injection | |
| Chlorpromazine | THORAZINE | rectal | suppositories | 30-800 mg/day |
| | | oral | capsules | (200-500 mg/day) |
| | | | solution | |
| | | | tablets | |
| | | parenteral | injection | |
| Fluphenazine | PROLIXIN | | | 0.5-40 mg/day |
| | | | | (1-5 mg/day) |
| Fluphenazine | PROLIXIN | parenteral | injection | (about one-half |
| decanoate | Decanoate | | | the dosage shown |
| | | | | for oral) |
| Fluphenazine | PROLIXIN | parenteral | injection | (same as above |
| enanthate | | | | |
| Fluphenazine | PROLIXIN | oral | elixer | |
| hydrochloride | | | solution | |
| | | parenteral | injection | |
| Thiothixene | NAVANE | oral | capsules | 6-60 mg/day |
| | | | | (8-30 mg/day) |
| Thiothixene | NAVANE | oral | solution | |
| hydrochloride | | parenteral | injection | |
| Trifluoperazine | STELAZINE | | | (2-40 mg/day) |
| Perphenazine | TRILAFON | oral | solution | 12-64 mg/day |
| | | | tablets | (16-64 mg/day) |
| | | parenteral | injection | |
| Perpehazine and | ETRAFON | oral | tablets | |
| Amitriptyline | TRIAVIL | | | |
| hydrochloride | | | | |
| Thioridazine | MELLARIL | oral | suspension | 150-800 mg/day |
| | | | solution | (100-300 mg/day) |
| | | | tablets | |
| Mesoridazine | | | | (30-400 mg/day) |
| Molindone | MOBAN | | | 50-225 mg/day |
| | | | | (15-150 mg/day) |
| Molindone | MOBAN | oral | solution | |
| hydrochloride | | | | |
| Loxapine | LOXITANE | | | 20-250 mg/day |
| | | | | (60-100 mg/day) |
| Loxapine | LOXITANE | oral | solution | |
| hydrochloride | | parenteral | injection | |
| Loxapine | LOXITANE | oral | capsules | |
| succinate | | | | |
| Pimozide | | | | (1-10 mg/day) |
| Flupenthixol | | | | |
| Promazine | SPARINE | | | |
| Triflupromazine | VESPRIN | | | |
| Chlorprothixene | TARACTAN | | | |
| Droperidol | INAPSINE | | | |
| Acetophenazine | TINDAL | | | |
| Prochlorperazine | COMPAZINE | | | |
| Methotrimeprazine | NOZINAN | | | |
| Pipotiazine | PIPOTRIL | | | |
| Aripiprazole | | | | |
| Hoperidone | | | | |

Examples of tradenames and suppliers of selected neuroleptic drugs are as follows clozapine (available under the tradename CLOZARIL®, from Mylan, Zenith Goldline, UDL, Novartis); olanzapine (available under the tradename ZYPREX®, from Lilly ziprasidone (available under the tradename GEODON®, from Pfizer); risperidone (available under the tradename RISPERDAL®, from Janssen); quetiapine fumarate (available under the tradename SEROQUEL®, from AstraZeneca); haloperidol (available under the tradename HALDOL®, from Ortho-McNeil); chlorpromazine (available under the tradename THORAZINE®, from Smith Kline Beecham (GSK); fluphenazine (available under the tradename PROLIXIN®, from Apothecon, Copley, Schering, Teva, and American Pharmaceutical Partners, Pasadena); thiothixene (available under the tradename NAVANE®; from Pfizer); trifluoperazine (10-[3-(4-methyl-1-piperazinyl)propyl]-2-(trifluoromethyl)phenothiazine dihydrochloride, available under the tradename STELAZINE®, from Smith Klein Beckman; perphenazine (available under the tradename TRILAFON®; from Schering); thioridazine (available under the tradename MELLARIL®; from Novartis, Roxane, HiTech, Teva, and Alpharma); molindone (available under the tradename MOBAN®, from Endo); and loxapine (available under the tradename LOXITANE®; from Watson). Furthermore, benperidol (Glianimon®), perazine (Taxilan®) or melperone (Eunerpan®)) may be used.

Other preferred neuroleptic drugs include promazine (available under the tradename SPARINE®), triflurpromazine (available under the tradename VESPRIN®), chlorprothixene (available under the tradename TARACTAN®), droperidol (available under the tradename INAPSINE®), acetophenazine (available under the tradename TINDAL®;), prochlorperazine (available under the tradename COMPAZINE®), methotrimeprazine (available under the tradename NOZINAN®), pipotiazine (available under the tradename PIPOTRIL®), ziprasidone, and hoperidone.

Particularly preferred neuroleptic agents for use in the invention are olanzapine, risperidone, quetiapine, aripiprazole, haloperidol, clozapine, ziprasidone and osanetant.

It will be appreciated by those skilled in the art that the compounds according to the invention may advantageously be used in conjunction with one or more other therapeutic agents, for instance, different antidepressant agents such as 5HT3 antagonists, serotonin agonists, NK-1 antagonists, selective serotonin reuptake inhibitors (SSRI), noradrenaline re-uptake inhibitors (SNRI), tricyclic antidepressants, dopaminergic antidepressants, H3 antagonists, 5HT1A antagonists, 5HT1B antagonists, 5HT1D antagonists, D1 agonists, M1 agonists and/or anticonvulsant agents, as well as atypical antipsychotic drugs and cognitive enhancers.

Suitable 5HT3 antagonists which may be used in combination of the compounds of the inventions include for example ondansetron, granisetron, metoclopramide. Suitable serotonin agonists which may be used in combination with the compounds of the invention include sumatriptan, rauwolscine, yohimbine, metoclopramide.

Suitable SSRIs which may be used in combination with the compounds of the invention include fluoxetine, citalopram, femoxetine, fluvoxamine, paroxetine, indalpine, sertraline, zimeldine.

Suitable SNRIs which may be used in combination with the compounds of the invention include venlafaxine and reboxetine.

Suitable tricyclic antidepressants which may be used in combination with a compound of the invention include imipramine, amitriptyline, clomipramine and nortriptyline.

Suitable dopaminergic antidepressants which may be used in combination with a compound of the invention include bupropion and amineptine.

Suitable anticonvulsant agents which may be used in combination of the compounds of the invention include for example divalproex, carbamazepine and diazepam.

Suitable atypical antipsychotic drugs which which may be used in combination of the compounds of the invention include for example risperidone, olanzapine, ziprasidone, aripiprazole and clozapine.

It will be appreciated that the compounds of the combination or composition may be administered simultaneously (either in the same or different pharmaceutical formulations), separately or sequentially.

In a further aspect of the invention, there is provided a compound of formula (I) as hereinbefore described or a salt or solvate thereof, for use in therapy.

In another aspect of the invention, there is provided a compound of formula (I) as hereinbefore described or a salt or solvate thereof, for use in the treatment of a disorder mediated by GlyT1.

As used herein, the term “a disorder mediated by GlyT1” refers to a disorder that may be treated by the administration of a medicament that alters the activity of the GlyT1 transporter. As hereinbefore described, the action of GlyT1 transporters affects the local concentration of glycine around NMDA receptors. As a certain amount of glycine is needed for the efficient functioning of NMDA receptors, any change to that local concentration can affect NMDA-mediated neurotransmission. As hereinbefore described, changes in NMDA-mediated neurotransmission have been implicated in certain neuropsychiatric disorders such as dementia, depression and psychoses, for example schizophrenia, and learning and memory disorders, for example attention deficit disorders and autism. Thus, alterations in the activity of the GlyT1 transporter are expected to influence such disorders.

The disorders mediated by GlyT1 referred to herein include neurological and neuropsychiatric disorders, including psychoses such as schizophrenia, dementia and other forms of impaired cognition such as attention deficit disorders and organic brain syndromes. Other neuropsychiatric disorders include drug-induced (phencyclidine, ketamine and other dissociative anesthetics, amphetamine and other psychostimulants and cocaine) psychosis, psychosis associated with affective disorders, brief reactive psychosis, schizoaffective psychosis, and psychosis NOS, “schizophrenia-spectrum” disorders such as schizoid or schizotypal personality disorders, or illness associated with psychosis (such as major depression, manic depressive (bipolar) disorder, Alzheimer's disease and post-traumatic stress syndrome), and NMDA receptor-related disorders such as autism, depression, benign forgetfulness, childhood learning disorders and closed head injury.

In a further aspect of the invention, there is provided a method of treating a mammal, including a human, suffering from or susceptible to a disorder mediated by GlyT1, which comprises administering an effective amount of a compound of formula (Ib) or a salt or solvate thereof:

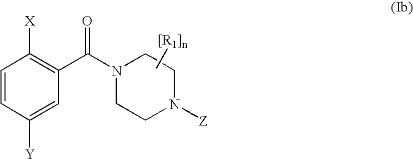

wherein
X is optionally substituted aryl or optionally substituted heteroaryl whereby when X is optionally substituted phenyl, the phenyl ring is not substituted at the meta or para position by methoxy, at the ortho position by chloro or at the para position by cyano;
Y is S(O)ₘR₂, wherein m is 1 or 2 and R₂ is optionally substituted C₁₋₆alkyl, optionally substituted C₃₋₇cycloalkyl, optionally substituted aryl, optionally substituted heteroaryl; or Y is a group —SO₂NR₃R₄ wherein R₃ and R₄ are independently hydrogen or optionally substituted C₁₋₆alkyl;
n is 0, 1 or 2, and R₁ is independently optionally substituted C₁₋₆alkyl; or the group [R₁]ₙ is a C₁₋₃alkylene group which forms a bridge across the piperazine ring; and
Z is an optionally substituted aryl or an optionally substituted heteroaryl.

All features and preferences for formula (I) as described above apply to compounds of formula (Ib) mutatis mutandis.

In another aspect of the invention, there is provided use of a compound of formula (Ib) as hereinbefore defined or a salt or solvate thereof in the preparation of a medicament for the treatment of a disorder mediated by GlyT1.

Preferably, the disorder mediated by GlyT1 to be treated by the use or method as hereinbefore described is a psychosis, including schizophrenia, dementia and attention deficit disorders, particularly schizophrenia.

As used herein, the term “effective amount” means that amount of a drug or pharmaceutical agent that will elicit the biological or medical response of a tissue, system, animal or human that is being sought, for instance, by a researcher or clinician.

Possible formulations include those suitable for oral, sub-lingual, buccal, parenteral (for example, subcutaneous, intramuscular, or intravenous), rectal, topical and intranasal administration and in forms suitable for administration by inhalation or insufflation (either through the mouth or nose). The most suitable means of administration for a particular patient will depend on the nature and severity of the conditions being treated and on the nature of the active compound, but, where possible, oral administration is preferred.

Formulations suitable for oral administration may be provided as discrete units, such as tablets, capsules, cachets, or lozenges, each containing a predetermined amount of the active compound; as powders or granules; as solutions or suspensions in aqueous or non-aqueous liquids; or as oil-in-water or water-in-oil emulsions.

Formulations suitable for sublingual or buccal administration include lozenges comprising the active compound and, typically, a flavoured base, such as sugar and acacia or tragacanth and pastilles comprising the active compound in an inert base, such as gelatin and glycerin or sucrose and acacia.

Formulations suitable for parenteral administration typically comprise sterile aqueous solutions containing a predetermined concentration of the active compound; the solution is preferably isotonic with the blood of the intended recipient. Although such solutions are preferably administered intravenously, they may also be administered by subcutaneous or intramuscular injection.

Formulations suitable for rectal administration are preferably provided as unit-dose suppositories comprising the active ingredient and one or more solid carriers forming the suppository base, for example, cocoa butter.

Formulations suitable for topical or intranasal application include ointments, creams, lotions, pastes, gels, sprays, aerosols and oils. Suitable carriers for such formulations include petroleum jelly, lanolin, polyethylene glycols, alcohols, and combinations thereof.

The formulations of the invention may be prepared by any suitable method, typically by uniformly and intimately admixing the active compound(s) with liquids or finely divided solid carriers, or both, in the required proportions and then, if necessary, shaping the resulting mixture into the desired shape.

For example, a tablet may be prepared by compressing an intimate mixture comprising a powder or granules of the active ingredient and one or more optional ingredients, such as a binder, lubricant, inert diluent, or surface active dispersing agent, or by moulding an intimate mixture of powdered active ingredient and inert liquid diluent.

Aqueous solutions for parenteral administration are typically prepared by dissolving the active compound in sufficient water to give the desired concentration and then rendering the resulting solution sterile and isotonic.

It will be appreciated that the precise dose administered will depend on the age and condition of the patient and the frequency and route of administration and will be at the ultimate discretion of the attendant physician. The compound may be administered in single or divided doses and may be administered one or more times, for example 1 to 4 times per day.

A proposed dose of the active ingredient for use according to the invention for oral, sub-lingual, parenteral, buccal, rectal, intranasal or topical administration to a human (of approximately 70 kg bodyweight) for the treatment of neurological and neuropsychiatric disorders mediated by a GlyT1 inhibitor, including schizophrenia, may be about 1 to about 1000 mg, preferably about 5 to about 500 mg, more preferably about 10 to about 100 mg of the active ingredient per unit dose which could be administered, for example, 1 to 4 times per day.

The invention is further illustrated by the following non-limiting examples.

### DESCRIPTION 1

### General Method for Suzuki Cross-Coupling Reactions on Methyl Benzoate Derivatives

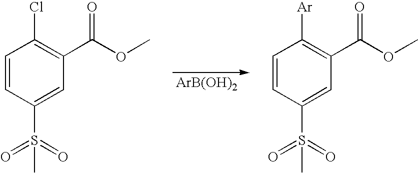

A reaction mixture of 2-chloro-5-methanesulfonyl-benzoic acid methyl ester (100 mg, 0.4 mmol, 1 equiv.), sodium bicarbonate (135 mg, 0.16 mmol, 4 equiv.), arylboronic acid (0.8 mmol, 2 equiv.), palladium(II) acetate (5 mg, 20 μmol, 0.05 equiv.) and triphenylphosphine (16 mg, 601 mmol, 0.15 equiv.) in a degassed mixture of 5:2 N,N-dimethylformamide/water (2.5 ml) under nitrogen atmosphere, was heated to 100° C. for 18 hrs. The cooled reaction mixture was filtered through a pad of silica and the cake was washed with dichloromethane. The filtrate was then concentrated in vacuo to give an oil, which was then re-dissolved in dichloromethane and washed with water. The organic layer was dried over anhydrous magnesium sulfate, filtered and concentrated in vacuo to give the crude product. Purification by flash chromatography (SiO₂) gave the pure compound.

The following compounds were prepared by the above general method:

### DESCRIPTION 2

### 4-Methanesulfonyl-biphenyl-2-carboxylic acid methyl ester

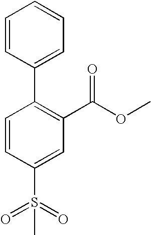

The title compound was prepared from 2-chloro-5-methanesulfonyl-benzoic acid methyl ester according to the procedure of Description 1.

d_{H} (400 MHz, CDCl₃) 8.40 (1H, d, ArH), 8.25 (1H, d, ArH), 8.09 (1H, d), 7.65 (1H, m, ArH), 7.57 (2H, d, ArH), 7.35 (2H, d), 3.70 (3H, OMe) and 3.15 (3H, s, SO₂Me).

### DESCRIPTION 3

### 4-Methanesulfonyl-4′-methoxy-biphenyl-2-carboxylic acid methyl ester

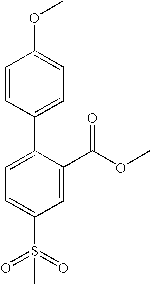

The title compound was prepared from 2-chloro-5-methanesulfonyl-benzoic acid methyl ester according to the procedure of Description 1.

d_{H} (400 MHz, CDCl₃) 8.35 (1H, d, ArH), 8.17 (1H, d, ArH), 8.05 (1H, d, ArH), 7.60 (2H, d, ArH), 6.99 (1H, d, ArH), 6.90 (1H, d, ArH), 3.85 (3H, OMe), 3.72 (3H, OMe) and 3.12 (3H, s, SO₂Me).

### DESCRIPTION 4

### 4-Methanesulfonyl-2′-methoxy-biphenyl-2-carboxylic acid methyl ester

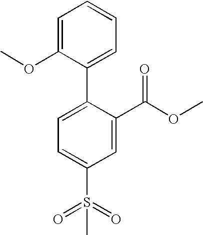

The title compound was prepared from 2-chloro-5-methanesulfonyl-benzoic acid methyl ester according to the procedure of Description 1.

LC/MS (ammonium bicarbonate ESI) Found 321 (M+1, T_{R} 1.90 min)

### DESCRIPTION 5

### 4-Methanesulfonyl-3′-methoxy-biphenyl-2-carboxylic acid methyl ester

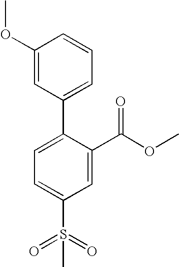

The title compound was prepared from 2-chloro-5-methanesulfonyl-benzoic acid methyl ester according to the procedure of Description 1.

d_{H} (400 MHz, CDCl₃) 8.35 (1H, d, ArH), 8.05 (1H, d, ArH), 7.60 (1H, d, ArH), 7.40 (2H, m, ArH), 6.90 (1H, m, ArH), 6.87 (1H, m, ArH), 3.82 (3H, OMe), 3.72 (3H, OMe) and 3.15 (3H, s, SO₂Me).

LC/MS (ammonium bicarbonate ESI) Found 321 (M+1, T_{R} 1.85 min)

### DESCRIPTION 6

### 2-Furan-3-yl-5-methanesulfonyl-benzoic acid methyl ester

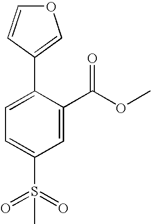

The title compound was prepared from 2-chloro-5-methanesulfonyl-benzoic acid methyl ester according to the procedure of Description 1.

d_{H} (400 MHz, CDCl₃) 8.30 (1H, d, ArH), 8.03 (1H, d, ArH), 7.67 (1H, d), 7.60 (1H, m, ArH), 7.50 (1H, d, ArH), 6.52 (1H, d, ArH), 3.90 (3H, OMe) and 3.08 (3H, s, SO₂Me).

### DESCRIPTION 7

### 5-Methanesulfonyl-2-pyridin-4-yl-benzoic acid methyl ester

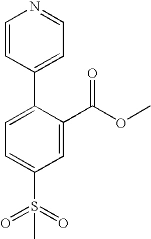

The title compound was prepared from 2-chloro-5-methanesulfonyl-benzoic acid methyl ester according to the procedure of Description 1.

d_{H} (400 MHz, CDCl₃) 8.70 (2H, d, ArH), 8.55 (1H, br s, ArH), 8.15 (1H, dd, ArH), 7.55 (1H, d, ArH), 7.27 (2H, d, ArH), 3.72 (3H, OMe) and 3.15 (3H, s, SO₂Me).

LC/MS (ammonium bicarbonate ESI) Found 292 (M+1, T_{R} 1.35 min)

### DESCRIPTION 8

### 2′-Chloro-4-methanesulfonyl-biphenyl-2-carboxylic acid methyl ester

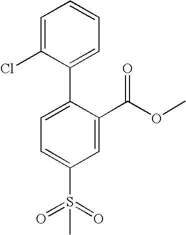

The title compound was prepared from 2-chloro-5-methanesulfonyl-benzoic acid methyl ester according to the procedure of Description 1.

d_{H} (400 MHz, CDCl₃) 8.50 (1H, br s, ArH), 8.12 (1H, d, ArH), 7.52 (1H, d, ArH), 7.45 (1H, m, ArH), 7.35 (2H, m, ArH), 7.30 (1H, m, ArH), 3.75 (3H, OMe), and 3.15 (3H, s, SO₂Me).

### DESCRIPTION 9

### 5-Methanesulfonyl-2-pyridin-3-yl-benzoic acid methyl ester

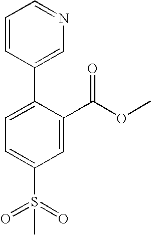

The title compound was prepared from 2-chloro-5-methanesulfonyl-benzoic acid methyl ester according to the procedure of Description 1.

### DESCRIPTION 10

### General Method for Suzuki Cross-Coupling Reactions on Benzoic Acid Derivatives

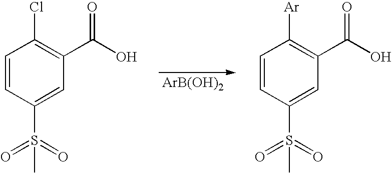

A reaction mixture of 5-methanesulfonyl-2-chlorobenzoic acid (100 mg, 0.43 mmol, 1 equiv.), sodium bicarbonate (145 mg, 0.17 mmol, 4 equiv.), arylboronic acid (0.86 mmol, 2 equiv.), palladium(II) acetate (5 mg, 20 mmol, 0.05 equiv.) and triphenylphosphine (17 mg, 65 μmol, 0.15 equiv.) in a degassed mixture of 5:2 N,N-dimethylformamide/water (2.5 ml) under nitrogen atmosphere, was heated to 100° C. for 18 hrs. The cooled reaction mixture was filtered through a pad of silica and the cake was washed with dichloromethane. The filtrate was then concentrated in vacuo to give an oil, which was then re-dissolved in dichloromethane and washed with water. The organic layer was dried over anhydrous magnesium sulfate, filtered and concentrated in vacuo to give the crude product. Purification by flash chromatography (SiO₂) gave the pure compound.

The following compounds were prepared by the above general method:

### DESCRIPTION 11

### 4′-Cyano-4-methanesulfonyl-biphenyl-2-carboxylic acid

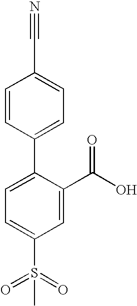

The title compound was prepared from 2-chloro-5-methanesulfonyl-benzoic acid methyl ester according to the procedure of Description 10.

d_{H} (400 MHz, CDCl₃) 8.59 (1H, s, ArH), 8.15 (1H, s, ArH), 7.75 (2H, d, ArH), 7.55 (1H, d, ArH), 7.45 (2H, d, ArH) and 3.15 (3H, s, SO₂Me).

LC/MS (Formic APCI) Found 300 (M−1, T_{R} 1.28 min)

### DESCRIPTION 12

### 4′-Chloro-4-methanesulfonyl-biphenyl-2-carboxylic acid

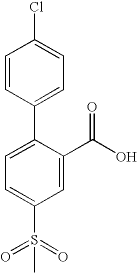

The title compound was prepared from 2-chloro-5-methanesulfonyl-benzoic acid methyl ester according to the procedure of Description 10.

d_{H} (400 MHz, CDCl₃) 8.55 (1H, br s, ArH), 8.12 (1H, dd, ArH), 7.55 (1H, d, ArH), 7.40 (2H, d, ArH), 7.30 (2H, d, ArH) and 3.15 (3H, s, SO₂Me).

LC/MS (Ammonium bicarbonate APCI) Found 309 (M−1, T_{R} 0.91 min)

### DESCRIPTION 13

### General Method for Hydrolysis of Benzoate Methyl Esters

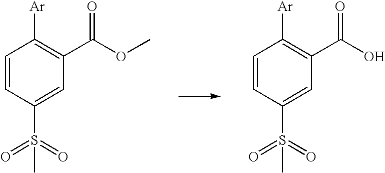

A reaction mixture of the ester (2.2 mmol), methanol (10 ml) and aqueous sodium hydroxide (10 ml, 2M) was heated to 70° C. for 18 hrs. The cooled reaction mixture was then diluted with water and ethyl acetate. The aqueous layer was acidified to pH 1 with aqueous hydrochloric acid (1M) and extracted with ethyl acetate. The organic layer was dried over anhydrous magnesium sulfate, filtered and concentrated in vacuo to give the crude product. Purification, if necessary, was generally by trituration to give the pure compound.

The following compounds were prepared by the above method:—

### DESCRIPTION 14

### 4-Methanesulfonyl-biphenyl-2-carboxylic acid

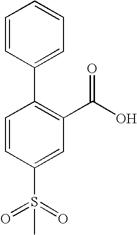

The title compound was prepared by the method described in Description 13.

d_{H} (400 MHz, CDCl₃) 8.50 (1H, d, ArH), 8.12 (1H, d, ArH), 7.62 (1H, m, ArH), 7.45 (3H, m, ArH), 7.35 (2H, d) and 3.15 (3H, s, SO₂Me).

LC/MS (Formic APCI) Found 275 (M−1, T_{R} 1.30 min).

### DESCRIPTION 15

### 4-Methanesulfonyl-4′-methoxy-biphenyl-2-carboxylic acid

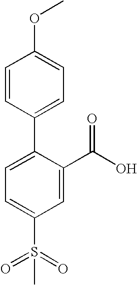

The title compound was prepared by the method described in Description 13.

d_{H} (400 MHz, CDCl₃) 8.47 (1H, br s, ArH), 8.10 (1H, dd, ArH), 7.55 (1H, m, ArH), 7.30 (2H, d, ArH), 6.96 (2H, d, ArH), 3.89 (3H, s, OMe) and 3.15 (3H, S, SO₂Me).

LC/MS (Formic ESI) Found 305 (M−1, T_{R} 1.33 min)

### DESCRIPTION 16

### 4-Methanesulfonyl-2′-methoxy-biphenyl-2-carboxylic acid

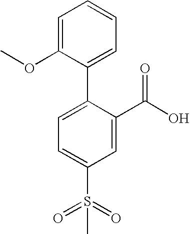

The title compound was prepared by the method described in Description 13.

d_{H} (400 MHz, CDCl₃) 8.50 (1H, br s, ArH), 8.10 (1H, dd, ArH), 7.55 (1H, d, ArH), 7.40 (1H, t, ArH), 7.27 (1H, m, ArH), 7.10 (1H, t, ArH), 6.92 (1H, d, ArH), 3.85 (3H, s, OMe) and 3.15 (3H, s, SO₂Me).

LC/MS (Formic ESI) Found 305 (M−1, T_{R} 1.30 min).

### DESCRIPTION 17

### 4-Methanesulfonyl-3′-methoxy-biphenyl-2-carboxylic acid

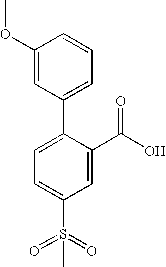

The title compound was prepared by the method described in Description 13.

d_{H} (400 MHz, CDCl₃) 8.47 (1H, br s, ArH), 8.10 (1H, dd, ArH), 7.55 (1H, d, ArH), 7.30 (1H, t, ArH), 6.92-6.85 (3H, m, ArH), 3.85 (3H, s, OMe) and 3.15 (3H, s, SO₂Me).

LC/MS (Ammonium bicarbonate ESI) Found 305 (M−1, T_{R} 0.83 min)

### DESCRIPTION 18

### 2-Furan-3-yl-5-methanesulfonyl-benzoic acid

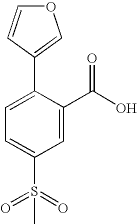

The title compound was prepared by the method described in Description 13.

d_{H} (400 MHz, CDCl₃) 8.45 (1H, br s, ArH), 8.09 (1H, dd, ArH), 7.68 (1H, s, ArH), 7.63 (1H, d, ArH), 7.48 (1H, s, ArH), 6.55 (1H, s, ArH) and 3.12 (3H, s, SO₂Me).

LC/MS (Formic ESI) Found 265 (M−1, T_{R} 1.06 min)

### DESCRIPTION 19

### 5-Methanesulfonyl-2-pyridin-4-yl-benzoic acid

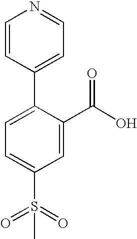

The title compound was prepared by the method described in Description 13.

### DESCRIPTION 20

### 2′-Chloro-4-methanesulfonyl-biphenyl-2-carboxylic acid

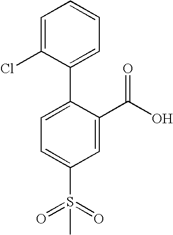

The title compound was prepared by the method described in Description 13.

Mixture of rotamers d_{H} (400 MHz, CDCl₃) 8.62+8.52 (1H, s, ArH), 8.18+8.12 (1H, s, ArH), 7.58+7.54 (1H, d, ArH), 7.52+7.42 (2H, m, ArH), 7.24 (2H, d, ArH) and 3.15+3.13 (3H, S, SO₂Me).

LC/MS (Formic ESI) Found 309 (M−1, T_{R} 1.45 min)

### DESCRIPTION 21

### 5-Methanesulfonyl-2-pyridin-3-yl-benzoic acid

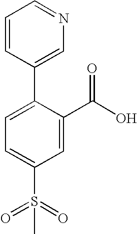

The title compound was prepared by the method described in Description 13.

### DESCRIPTION 22

### General Method for Amide Coupling Reaction

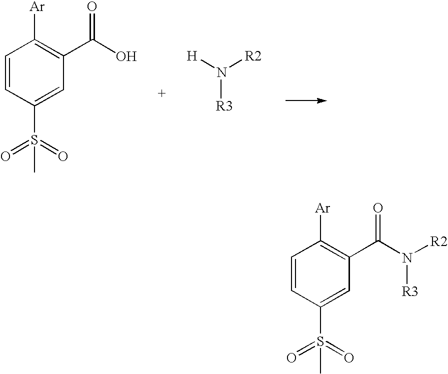

A reaction mixture of amine (0.19 mmol, 1.2 equiv.), acid (0.16 mmol, 1 equiv.) and EDCI (0.32 mmol, 2 equiv.) in dry dichloromethane was stirred at 35° C. until the reaction was complete. The reaction mixture was then diluted with dichloromethane and washed with aqueous hydrochloric acid (1M) followed by saturated aqueous sodium bicarbonate. The organic layer was then dried over anhydrous magnesium sulfate, filtered and concentrated in vacuo to give the crude product. Purification, if necessary, was by flash chromatography (SiO₂) to give the pure compound.

### DESCRIPTION 23

### Methyl 4′-fluoro-4-(methylsulfonyl)-2-biphenylcarboxylate

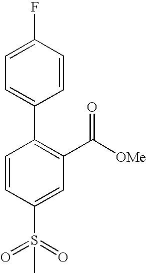

The title compound was prepared by the procedure described in Description 1.

d_{H} (400 MHz, CDCl₃) 8.29 1H, d, ArH), 8.18 (1H, dd, ArH), 7.73 (1H, d, ArH), 7.41 (2H, dd, ArH), 7.31 (2H, t, ArH), 3.69 (3H, s), 3.37 (3H, s).

### DESCRIPTION 24

### 4′-Fluoro-4-(methylsulfonyl)-2-biphenylcarboxylic acid

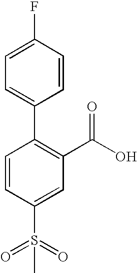

The title compound was prepared from methyl 4′-fluoro-4-(methylsulfonyl)-2-biphenylcarboxylate according to the procedure described in Description 13.

LC/MS Found 293 (ESI) (M−1).

### DESCRIPTION 25

### Methyl 2′,4′-difluoro-4-(methylsulfonyl)-2-biphenylcarboxylate

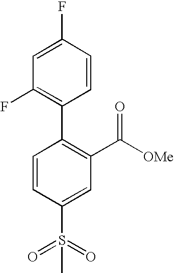

The title compound was prepared by the procedure described in Description 1.

### DESCRIPTION 26

### 2′,4′-Difluoro-4-(methylsulfonyl)-2-biphenylcarboxylic acid

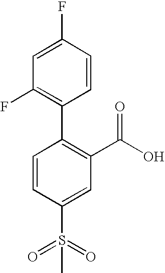

The title compound was prepared from methyl 2′,4′-difluoro-4-(methylsulfonyl)-2-biphenylcarboxylate according to the procedure described in Description 13.

LC/MS Found 311 (ESI) (M−1).

### DESCRIPTION 27

### Methyl 2′-fluoro-4-(methylsulfonyl)-2-biphenylcarboxylate

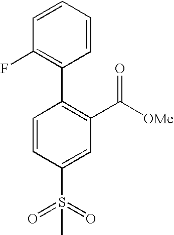

The title compound was prepared by the procedure described in Description 1.

### DESCRIPTION 28

### 2′-Fluoro-4-(methylsulfonyl)-2-biphenylcarboxylic acid

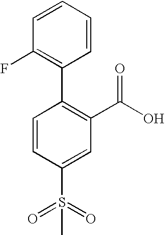

The title compound was prepared from methyl 2′-fluoro-4-(methylsulfonyl)-2-biphenylcarboxylate according to the procedure described in Description 13.

LC/MS Found 293 (ESI) (M−1).

### DESCRIPTION 29

### Methyl 3′-fluoro-4-(methylsulfonyl)-2-biphenylcarboxylate

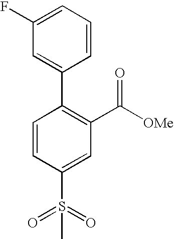

The title compound was prepared by the procedure described in Description 1.

### DESCRIPTION 30

### 3′-Fluoro-4-(methylsulfonyl)-2-biphenylcarboxylic acid

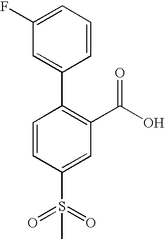

The title compound was prepared from methyl 3′-fluoro-4-(methylsulfonyl)-2-biphenylcarboxylate according to the procedure described in Description 13.

LC/MS Found 293 (ESI) (M−1).

### DESCRIPTION 31

### 1-[3-Fluoro-4-(1-piperazinyl)phenyl]ethanone

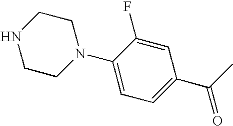

A solution of 3′,4′-difluoroacetophenone (14.3 g, 0.092 mol) in acetonitrile (150 ml) was treated with piperazine (27.6 g, 0.32 mol) and heated at reflux for 18 h. The mixture was allowed to cool and partitioned between EtOAc and water. The organics were further washed with water (x2), dried (Na₂SO₄) and evaporated in vacuo to the title compound (17.6 g, 86%).

LC/MS Found 223 (ESI) (M+1)

### DESCRIPTION 32

### 4-(Methylsulfinyl)-2-biphenylcarboxylic acid

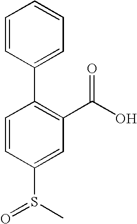

A mixture of methyl 2-chloro-5-(methylsulfinyl)benzoate (750 mg), phenylboronic acid ((0.79 g), sodium bicarbonate (1.1 g), palladium acetate (80 mg), triphenylphosphine (160 mg) DMF (14 ml) and water (5.5 ml) was heated at 100° under argon for 6.5 hours. The cooled solution was filtered through celite and washed with DCM. The filtrate was concentrated under reduced pressure to give an oil which was redissolved in ethyl acetate and washed with water (2×). Organic layer dried and evaporated to give a crude product which was chromatographed on silica gel. Elution with 20-100% ethyl acetate in pentane, gave desired product as a brown solid (0.74 g). LC/MS Found 275 (ESI) (M+1).

A solution of the methyl ester in methanol (5 ml) containing 2M sodium hydroxide (1.35 ml) was heated to 70° for 18 hours. The volatile components were removed under reduced pressure and the residue partitioned between water (5 ml) and ethyl acetate. The aqueous layer was acidified to pH 1 by addition of 2M hydrochloric acid and extracted (2×) with ethyl acetate. Combined organic layers evaporated to afford the desired product as a crisp foam (0.54 g). LC/MS Found 261 (ESI) (M+1).

### DESCRIPTION 33

### Methyl 2-chloro-5-[(methylamino)sulfonyl]benzoate

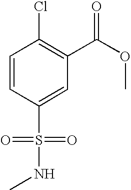

A stirred solution of 2M methylamine in THF (2.4 ml) was treated with 2-chloro-5-(chlorosulfonyl)benzoic acid (300 mg) and the resulting mixture stirred for 40 minutes. The solvent was then removed under reduced pressure to afford a gum. This was treated with a solution of excess HCl gas in methanol and stirred for 18 hours. Removal of the solvent gave a crude product which was chromatographed on silica gel. Elution with 20-100% ethyl acetate in pentane gave the methyl ester as a white solid (231 mg). LC/MS Found 262 (ESI) (M−H).

### DESCRIPTION 34

### 4-[(Methylamino)sulfonyl]-2-biphenylcarboxylic acid

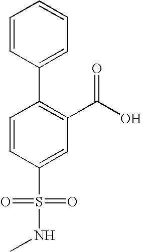

Prepared as described for 4-(methylsulfinyl)-2-biphenylcarboxylic acid. Obtained product as a white solid. LC/MS Found 290 (ESI) (M−H).

### DESCRIPTION 35

### 1,1-Dimethylethyl 4-[4-(trifluoroacetyl)phenyl]-1-piperazinecarboxylate

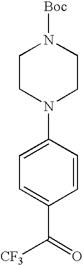

4-Bromo-trifluoroacetophenone (1.51 g), N-Boc piperazine (1.11 g), caesium carbonate (2.91 g), palladium acetate (120 mg) and BINAP (0.555 g) were heated in 1,4-dioxane (50 ml) at 100° under argon for 24 hours. After cooling, the precipitate was removed by filtration and the filtrate evaporated at reduced pressure. The residue was partitioned between ethyl acetate and water and the aqueous layer extracted with further ethyl acetate. The combined organic layers were dried (MgSO4) and evaporated. Chromatography on silica gel eluting with 0-100% ethyl acetate/pentane afforded the desired product as a pale solid (1.40 g).

### DESCRIPTION 36

### 2,2,2-Trifluoro-1-[4-(1-piperazinyl)phenyl]ethanone

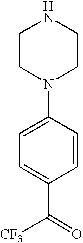

1,1-dimethylethyl 4-[4-(trifluoroacetyl)phenyl]-1-piperazinecarboxylate (0.84 g) in trifluoroacetic acid (5 ml) was stirred at ambient temperature for 3 hours. The solvent was then removed and the residue chromatographed on silica gel. Elution with 0-10% (9:1 MeOH:ammonia) in DCM gave the desired product as a pale foam (0.76 g).

### DESCRIPTION 37

### 2,2,2-Trifluoro-1-[4-(1-piperazinyl)phenyl]ethanol

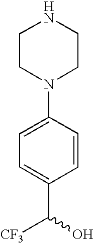

A solution of 2,2,2-trifluoro-1-[4-(1-piperazinyl)phenyl]ethanone (100 mg) in methanol (2 ml) under argon was cooled in ice and treated with sodium borohydride (15 mg). After 30 minutes at ambient temperature the solvent was removed and the residue partitioned between ethyl acetate and saturated NaHCO3 solution. The organic layer was dried and evaporated to give the desired product as a pale solid (100 mg). LC/MS (ESI) found 261 (M+H).

### DESCRIPTION 38

### 1,1,1,3,3,3-Hexafluoro-2-[4-(1-piperazinyl)phenyl]-2-propanol

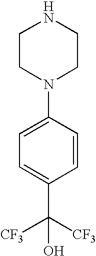

A suspension of 2-(4-aminophenyl)-1,1,1,3,3,3-hexafluoro-2-propanol (1.0 g), bis-(2-chloroethyl)amine hydrochloride (0.69 g) and potassium carbonate (0.53 g) in 2-butoxyethanol (5 ml), was heated at 180° for 24 hours. Diluted with ethyl acetate and washed with saturated NaHCO3 solution and brine. Organic layer dried and evaporated to give crude product. Chromatography on silica gel gave, after elution with 0-20% methanol in DCM, the desired product as a brown solid (0.54 g). LC/MS (ESI) found 329 (M+H).

### DESCRIPTION 39

### 1,1-Dimethylethyl 4-(4-acetyl-3,5-dichlorophenyl)-1-piperazinecarboxylate

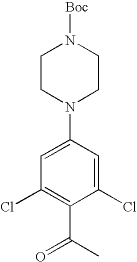

A solution of 1.4M methylmagnesium bromide in THF (29 ml) was stirred under argon at 50° and a solution of 2,4,6-trichlorobenzoyl chloride (2.00 g) in dry toluene (2 ml) was added over 15 minutes. Stirring was continued for a further 30 minutes and the cooled (0°) reaction mixture was then treated with ice and diluted with ether and 2M hydrochloric acid. Organic layer dried and evaporated to afford 2,4,6-trichloroacetophenone as an oil (1.78 g). This material (1.0 g) was then treated as in Description 35 to afford crude title compound. Chromatography on silica gel eluting with 0-50% ethyl acetate/pentane gave desired product as a yellow gum (81 mg). LC/MS (ESI) found 273 (MH-100).

### DESCRIPTION 40

### 1-[2,6-Dichloro-4-(1-piperazinyl)phenyl]ethanone

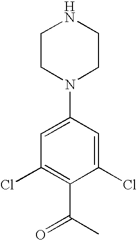

1,1-dimethylethyl 4-(4-acetyl-3,5-dichlorophenyl)-1-piperazinecarboxylate (81 mg) was treated with trifluoroacetic acid (0.9 ml) and water (0.1 ml) and stirred for 1 hour. The volatile components were removed under reduced pressure to afford the desired product as a brown gum.

### DESCRIPTION 41

### 2,6-Dichloro-4-(1-piperazinyl)benzaldehyde

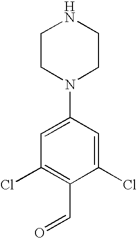

A solution of 2,4,6-trichlorobenzyl alcohol (0.98 g) in chloroform (40 ml) was treated with manganese dioxide (3.2 g) and sodium chloride (3.2 g) and heated to reflux for 18 hours. The solids were removed by filtration and the filtrate evaporated to afford crude product. Chromatography on silica gel, eluting with 0-50% ethyl acetate/pentane gave 2,4,6-trichlorobenzaldehyde (320 mg). This was treated as described in Descriptions 35 and 36 to afford the desired product as a yellow oil (70 mg).

### DESCRIPTION 42

### 4-(4-{([4′-Fluoro-4-(methylsulfonyl)-2-biphenyl]carbonyl}-1-piperazinyl)aniline

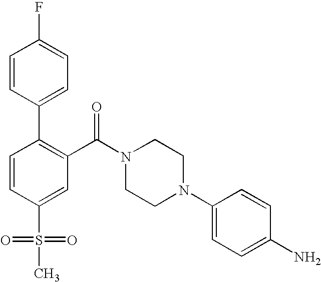

A solution of 1-{[4′-fluoro-4-(methylsulfonyl)biphenyl-2-yl]carbonyl}-4-(4-nitrophenyl)piperazine (2.46 g, 5.1 mmol) in ethanol (50 ml) was hydrogenated over 10% palladium on carbon (50% aq. paste) at atmospheric pressure/room temp. for 40 h. The mixture was filtered through celite and the filtrate evaporated in vacuo to a pale purple solid 2.4 g, quantitative. LC/MS (ESI) Found 454 (M+1).

### DESCRIPTION 43

### 3-Fluoro-4-(4-{[4′-fluoro-4-(methylsulfonyl)-2-biphenylyl]carbonyl}-1-piperazinyl)aniline

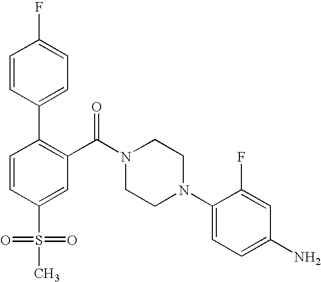

The title compound was prepared from 1-{[4′-fluoro-4-(methylsulfonyl)biphenyl-2-yl]carbonyl}-4-(2-fluoro-4-nitrophenyl)piperazine (800 mg, 1.6 mmol) in a similar manner to that described for Description 42 to afford a white solid 750 mg, 99%. LC/MS (ESI) Found 472 (M+1)

### DESCRIPTION 44

### N-Methyl-N-[4-(1-piperazinyl)phenyl]acetamide

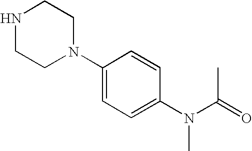

A solution of N-(4-bromophenyl)-N-methylacetamide (Organic Letters (2003), 5(8), 1265-1267; 230 mg, 1.0 mmol), cesium carbonate (489 mg, 1.5 mmol), rac-BINAP (93 mg, 0.15 mmol), palladium acetate (22 mg, 0.1 mmol) and piperazine (1.7 g, 20 mmol) in dioxan (10 ml) was heated at 100° C. under argon for 18 h. The dioxan was removed in vacuo and the residue partitioned between 2N HCl and EtOAc. The aqueous layer was basified (K₂CO₃) and extracted with MDC. The organic extracts were dried through PhaseSep cartridge and evaporated in vacuo to a pale yellow solid 152 mg, 65%).

δ_{H} (400 MHz, CDCl₃) 7.04 (2H, d), 6.93 (2H, d), 3.22 (3H, s), 3.16 (4H, m), 3.04 (4H, m), 1.86 (3H, s).

LC/MS (ESI) Found 234 (M+1)

### DESCRIPTION 45

### 1-[4-(1-piperazinyl)-2-(trifluoromethyl)phenyl]ethanone

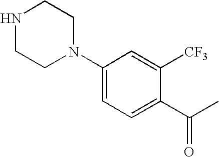

A solution of 1-[4-fluoro-2-(trifluoromethyl)phenyl]ethanone (4.83 g, 0.023 mol) in acetonitrile (150 ml) was treated with piperazine (7.1 g, 0.082 mol) and the mixture heated at reflux under argon for 20 h. On cooling the mixture was partitioned between water and EtOAc. The organics were washed further with water (x2), dried (Na₂SO₄) and evaporated in vacuo to a yellow solid 5.8 g, 93%.

δ_{H} (400 MHz, CDCl₃) 7.55 (1H, d), 7.18 (1H, d), 6.96 (1H, dd), 3.30 (4H, m), 3.03 (4H, m), 2.54 (3H, s).

LC/MS (ESI) Found 273 (M+1)

### DESCRIPTION 46

### 1-[2-Chloro-4-(1-piperazinyl)phenyl]ethanone

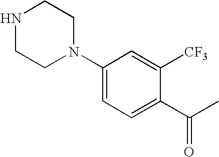

The compound was prepared from 1-(2-chloro-4-fluorophenyl)ethanone (4.8 g, 0.028 mol) by the procedure described in Description 45 to afford the title compound 6.4 g, 96%.

LC/MS (ESI) Found 239/241 (M+1)

### DESCRIPTION 47

### Cyclopropyl[3-fluoro-4-(1-piperazinyl)phenyl]methanone

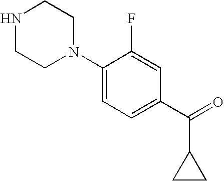

The compound was prepared from (4-bromo-3-fluorophenyl)(cyclopropyl)methanone (2.0 g, 8.23 mmol) in a similar manner to that for Description 44 to afford the title compound 1.8 g, 89%.

LC/MS (ESI) Found 249 (M+1).

### DESCRIPTION 47a

### 1-(3,5-Dichlorophenyl)-4-{[4′-fluoro-4-(methylsulfonyl)-2-biphenylyl]carbonyl}piperazine

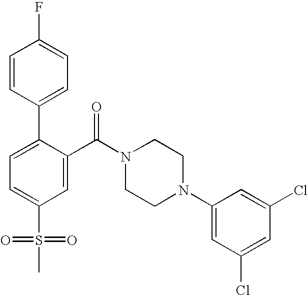

To a stirred solution of 4′-fluoro-4-(methylsulfonyl)-2-biphenylcarboxylic acid (0.294 g, 1 mmol) and 1-(3,5-dichlorophenyl)piperazine (0.231 g, 1 mmol) in dichloromethane (15 ml) was added EDC (0.192 g, 1 mmol) followed by 1-hydroxybenzotriazole (0.01 g, 0.07 mmol). After 1.5 h the reaction mixture was washed with saturated sodium hydrogen carbonate (50 ml) and the organics dried (Na₂SO₄) and evaporated in vacuo. The residue was purified by chromatography on silica gel eluting with 0-100% ethyl acetate in pentane gradient to afford the title compound as a colourless solid (0.48 g, 94%). LC/MS (ESI) Found 507 (M+1).

### DESCRIPTION 48

### 1,1-Dimethylethyl 4-(2-chloro-4-formylphenyl)-1-piperazinecarboxylate

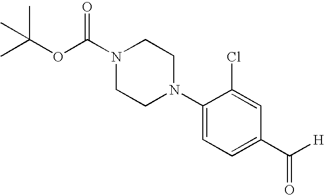

1,1-dimethylethyl 1-piperazinecarboxylate (6.5 g) was added in one portion to a stirred solution of 3-chloro-4-fluorobenzaldehyde (5 g) in anhydrous DMF (80 ml) under argon. Potassium carbonate (6.6 g) was added and the mixture heated at 100° C. for 20 h, cooled and evaporated in vacuo. The residue was partitioned between ethyl acetate (300 ml) and water (300 ml) and the organic layer washed with water (300 ml), dried (Na₂SO₄) and evaporated. The residue was purified by chromatography on silica gel eluting with 0-25% ethyl acetate in pentane gradient to afford the title compound (5.67 g, 56%). LC/MS (ESI) Found 325 (M+1).

### DESCRIPTION 49

### 1,1-Dimethylethyl 4-(3-chloro-4-formylphenyl)-1-piperazinecarboxylate

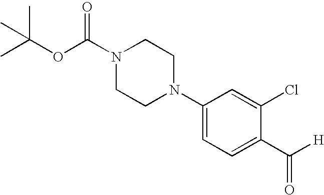

The title compound was prepared from 2-chloro-4-fluorobenzaldehyde (6.5 g) using the method of Description 48 (8.55 g, 83%). LC/MS (ESI) Found 325 (M+1).

### DESCRIPTION 50

### 1,1-Dimethylethyl 4-(2,6-difluoro-4-formylphenyl)-1-piperazinecarboxylate

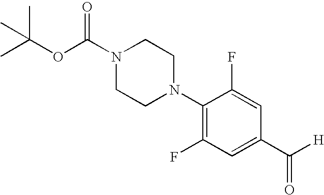

The title compound was prepared from 3,4,5-trifluorobenzaldehyde (7.1 g) using the method of Description 48 (3.75 g, 26%). δ_{H} (400 MHz, CDCl₃) 9.79 (1H, s, CHO), 7.39-7.36 (2H, m, ArH), 3.57-3.54 (4H, m), 3.30 (4H, m), 1.49 (9H, s).

### DESCRIPTION 51

### 3-Chloro-4-(1-piperazinyl)benzaldehyde

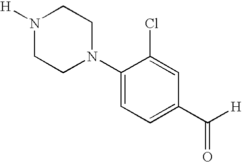

A solution of 1,1-dimethylethyl 4-(2-chloro-4-formylphenyl)-1-piperazinecarboxylate (2 g) and trifluoroacetic acid (10 ml) in DCM (80 ml) was stirred at room temperature for 2 h, evaporated in vacuo and the residue partitioned between DCM (200 ml) and saturated sodium bicarbonate (200 ml). The aqueous layer was extracted with DCM (200 ml) and the combined organics dried (Na₂SO₄) and evaporated to afford the title compound (1.35 g, 97%). LC/MS (ESI) Found 225 (M+1).

### DESCRIPTION 52

### 2-Chloro-4-(1-piperazinyl)benzaldehyde

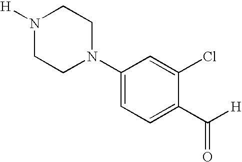

The title compound was prepared from 1,1-dimethylethyl 4-(3-chloro-4-formylphenyl)-1-piperazinecarboxylate (2 g) using the method of Description 51 (1.34 g, 97%). LC/MS (ESI) Found 225 (M+1).

### DESCRIPTION 53

### 3,5-Difluoro-4-(1-piperazinyl)benzaldehyde

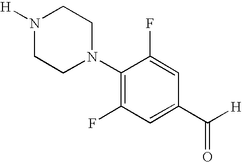

The title compound was prepared from 1,1-dimethylethyl 4-(2,6-difluoro-4-formylphenyl)-1-piperazinecarboxylate (2 g) using the method of Description 51 (1.34 g, 97%). LC/MS (ESI) Found 227 (M+1).

### DESCRIPTION 54

### 2,6-Dichloro-4-(4-{[4-(methylsulfonyl)-2-biphenylyl]carbonyl}-1-piperazinyl)phenol

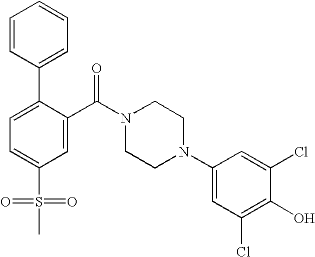

A mixture of 2′-[4-(4-Methoxy-3,5-dichloro-phenyl)-piperazine-1-carbonyl]-4′-methanesulfonyl-biphenyl (4 g) and 48% aqueous hydrobromic acid (200 ml) was heated at 105° C. for 18 h, cooled and evaporated to dryness in vacuo. The residue was partitioned between ethyl acetate and saturated sodium bicarbonate, the aqueous layer extracted with ethyl acetate and combined organics dried (Na₂SO₄) and evaporated. Chromatography on silica gel eluting with 0-70% ethyl acetate in pentane afforded the title compound as a beige solid (3.4 g, 87%). LC/MS (ESI) Found 505 (M+1).

### DESCRIPTION 55

### 1-{4-[(4-Bromobutyl)oxy]-3,5-dichlorophenyl}-4-{[4-(methylsulfonyl)-2-biphenylyl]carbonyl}piperazine

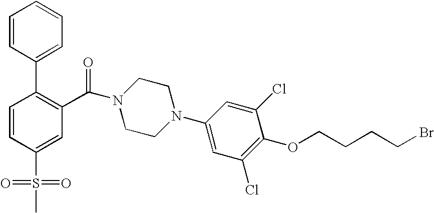

60% Sodium hydride in mineral oil (0.016 g) was added in one portion to a stirred solution of 2,6-Dichloro-4-(4-{[4-(methylsulfonyl)-2-biphenylyl]carbonyl}-1-piperazinyl)phenol (0.16 g) in anhydrous dimethylformamide (2 ml) at room temperature under argon. After 0.5 h this solution was added dropwise to a stirred solution of 1,4-dibromobutane (0.685 g) in anhydrous dimethylformamide (3 ml). After 18 h the mixture was diluted with diethyl ether (200 ml), washed with water (2×100 ml) then brine (50 ml), dried (Na₂SO₄) and the organic solvents evaporated in vacuo. Chromatography on silica gel eluting with 0-70% ethyl acetate in pentane gradient gave the title compound as a colourless foam (0.14 g, 69%). LC/MS (ESI) Found 639 (M+1).

### DESCRIPTION 56

### 2,6-Difluoro-4-(4-{[4-(methylsulfonyl)-2-biphenylyl]carbonyl}-1-piperazinyl)phenol

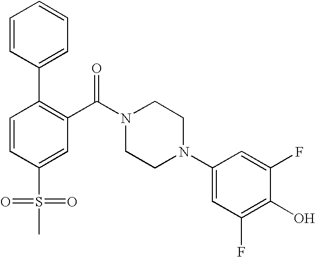

The title compound was prepared from 1-[3,5-difluoro-4-(methyloxy)phenyl]-4-{[4-(methylsulfonyl)-2-biphenylyl]carbonyl}piperazine (0.14 g) using the method of Description 54, (0.114 g, 84%). LC/MS (ESI) Found 473 (M+1).

### DESCRIPTION 57

### Methyl 5-(methylsulfonyl)-2-(3-thienyl)benzoate

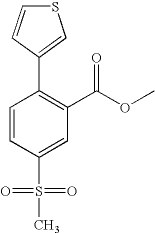

The title compound was prepared by the procedure Description 1.

d_{H} (400 MHz, CDCl₃) 8.30 (1H, d, ArH), 8.20 (1H, dd, ArH), 7.61 (1H, d, ArH), 7.35 (2H, dd, ArH), 7.10 (1H, dd, ArH), 3.8 (3H, s), 3.10 (3H, s).

### DESCRIPTION 58

### 5-(Methylsulfonyl)-2-(3-thienyl)benzoic acid

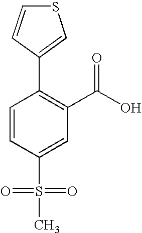

The title compound was prepared from methyl 5-(methylsulfonyl)-2-(3-thienyl)benzoate according to the procedure described in Description 13.

LC/MS (ESI) Found 305.8 (M+23)

### DESCRIPTION 59

### Methyl 5-(methylsulfonyl)-2-(2-thienyl)benzoate

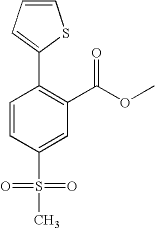

The title compound was prepared by the procedure described in Description 1.

d_{H} (400 MHz, CDCl3) 8.25 (1H, d, ArH), 8.0 (1H, dd, ArH), 7.7 (1H, dd, ArH), 7.44 (1H, dd, ArH), 7.13 (2H, m, ArH), 3.80 (3H, s), 3.10 (3H, s).

### DESCRIPTION 60

### 5-(methylsulfonyl)-2-(2-thienyl)benzoic acid

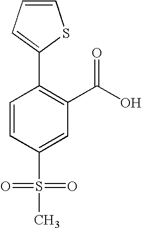

The title compound was prepared from methyl 5-(methylsulfonyl)-2-(2-thienyl)benzoate according to the procedure described in Description 13.

LC/MS Found 265.02 (ESI) (M−18)

### DESCRIPTION 61

### 1,1-Dimethylethyl 4-[3,5-difluoro-4-(methyloxy)phenyl]-1-piperazinecarboxylate

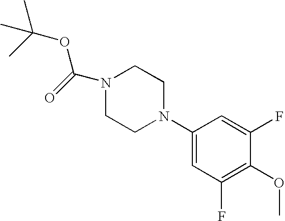

A mixture of 4-bromo-2,6-difluorophenyl methyl ether (5.0 g), 1,1-dimethylethyl 1-piperazinecarboxylate (5.0 g), 2-biphenylyl[bis(1,1-dimethylethyl)]phosphane (0.666 g), palladium acetate (0.246 g), sodium tert-butoxide (3.01 g) in toluene (30 ml) was heated at 90° C. for 16 hours. After cooling, The reaction mixture was diluted with diethyl ether, filtered through a pad of celite. Then concentrated in vacuo to give a black oil. The desired product was isolated by column chromatography on silica eluting with a gradient of 5 to 100% ethyl acetate in pentane. The title compound was obtained as a solid, (90% yield), LC/MS (ESI) Found 229 (M−BOC+1).

### DESCRIPTION 62

### 1-[3,5-Difluoro-4-(methyloxy)phenyl]piperazine

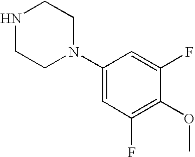

1,1-dimethylethyl 4-[3,5-difluoro-4-(methyloxy)phenyl]-1-piperazinecarboxylate (7.43 g) was dissolved in dichloromethane (50 ml). The solution was cooled in an ice bath. Then TFA (20 ml) was added dropwise. The resulting mixture was stirred at room temperature under argon for 16 hours. The reaction solution was evaporated to small volume at reduced pressure and partitioned between cold sodium bicarbonate solution and ethyl acetate. The aqueous solution was extracted twice with ethyl acetate and combined organic extracts were dried (Na₂SO₄) and evaporated. A brown solid obtained, (100% yield), LCMS (ESI) Found 229 (M+1).

### DESCRIPTION 63

### 1-[3-Chloro-5-fluoro-4-(methyloxy)phenyl]piperazine

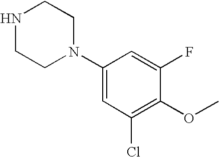

The title compound was prepared according to the procedure described in Descriptions 61 and 62.

### DESCRIPTION 64

### 1-[6-(1-piperazinyl)-3-pyridinyl]ethanone

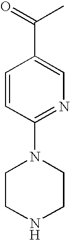

The title compound was prepared from 1-(6-chloro-3-pyridinyl)ethanone and piperazine according to the procedure described in J. Med. Chem. 1999, Vol 42, No: 14 p. 2577. (90% yield) LC/MS (ESI) Found 206.2 (M+1)

### DESCRIPTION 65

### 4-[4-(1H-pyrazol-1-yl)phenyl]-1-Boc-piperazine

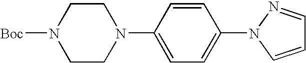

A mixture of 1-Boc-piperazine (500 mg, 1.45 mmol), Cs₂CO₃ (641 mg, 1.97 mmol), Pd(OAc)₂ (29 mg, 0.13 mmol) and racemic BINAP (125 mg, 0.20 mmol) in 1,4-dioxane (5 mL) was stirred vigorously for 20 min and 1-(4-iodophenyl)-1H-pyrazole (357 mg, 1.32 mmol) added. The reaction mixture was heated under reflux for 70 h and then cooled to room temperature. After filtering through celite, the volatile components were removed in vacuo and the product dissolved in water (25 mL). The aqueous was extracted with EtOAc (3×25 mL) and the combined organic layers dried (MgSO₄) and the solvent removed in vacuo. Purification by column chromatography (Flashmaster II, 50 g Isolute flash column, gradient elution 100% pentane to 20% EtOAc over 4 min, 20% EtOAc to 30% EtOAc over 16 min, 30% EtOAc to 100% EtOAc over 10 min) gave 4-[4-(1H-pyrazol-1-yl)phenyl]-1-Boc-piperazine (275 mg, 64%) as an off white solid. δ_{H} (400 MHz) 7.83-7.82 (1H, m), 7.69-7.68 (1H, m), 7.58-7.55 (2H, m), 6.99-6.96 (2H, m), 6.43-6.42 (1H, m), 3.59 (4H, t, J 5.1), 3.15 (4H, t, J 5.1), 1.49 (9H, s, C(CH₃)₃); LC/MS (Ammonium bicarbonate ES+) Found 229 (M−Boc+H, T_{R} 2.90 min).

### DESCRIPTION 66

### 1-[4-(1H-pyrazol-1-yl)phenyl]piperazine (93)

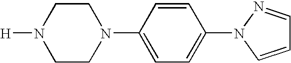

TFA (620 μL; 8.35 mmol) was added dropwise over 30 s to a stirring solution of the pyrazole 4-[4-(1H-pyrazol-1-yl)phenyl]-1-Boc-piperazine (275 mg, 0.84 mmol) in DCM (10 mL). Stirring was continued at room temperature for 114 h and the volatile components removed in vacuo. The TFA salt was converted to the free base by SCX (loaded directly, washed with MeOH, stationary phase removed using NH3 in MeOH (1 M)) and the solvent removed in vacuo to give the amine 1-[4-(1H-pyrazol-1-yl)phenyl]piperazine (150 mg, 79%) as an off white solid. δ_{H} (400 MHz) 8.04-8.03 (1H, m), 7.67-7.66 (1H, m), 7.57-7.53 (2H, m), 7.06-7.02 (2H, m), 6.48-6.47 (1H, m), 3.17-3.15 (4H, m), 2.99-2.96 (4H, m); LC/MS (Ammonium bicarbonate ES+) Found 229 (M+H, T_{R} 1.22 min).

### DESCRIPTION 67

### 4-[4-(1-Methyl-1H-pyrazol-3-yl)phenyl]-1-Boc-piperazine

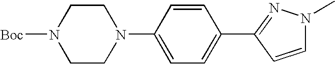

Methyl iodide (100 μL, 1.61 mmol) was added in one portion to a stirring solution of the 1,1-dimethylethyl 4-[4-(1H-pyrazol-3-yl)phenyl]-1-piperazinecarboxylate (104 mg, 0.47 mmol) and K₂CO₃ (140 mg, 1.01 mmol) in acetone (1.5 mL). Stirring was continued at room temperature for 120 h and the reaction quenched with NH₃ in MeOH (20 mL, 1 M). The volatile components were removed in vacuo and the product dissolved in water (10 mL), extracting with Et₂O (3×10 mL). The combined organic layers dried (MgSO₄) and reduced in vacuo. Purification by column chromatography (Biotage Horizon, 12+M Biotage column, gradient elution 100% pentane for 120 mL, 100% pentane to 20% EtOAc over 24 mL, 20% EtOAc for 210 mL) to give a 4:1 mixture of the methylated pyrazole regioisomers tert-butyl-4-[4-(1-methyl-1H-pyrazol-3-yl)phenyl]-1-piperazinecarboxylate and tert-butyl-4-[4-(1-methyl-1H-pyrazol-5-yl)phenyl]-1-piperazinecarboxylate (63 mg, 60%) as a white solid. δ_{H} (400 MHz, 4:1 mixture of regioisomers) 7.76-7.64ₘₐⱼ/7.60-7.58ₘᵢₙ (2H, m), 7.52-7.49ₘₐⱼ/7.29-7.28ₘᵢₙ (2H, m), 7.37ₘₐⱼ/7.26ₘᵢₙ (1H, d, J 2.2), 6.51ₘₐⱼ/6.30ₘᵢₙ (1H, d, J 2.2), 3.94ₘₐⱼ/3.88ₘᵢₙ (3H, s, NCH₃); LC/MS (Ammonium bicarbonate ES+) Found 238 (M+H, T_{R} 2.73 min).

### DESCRIPTION 68

### 4-[4-(Methylthio)phenyl]piperazine-1-carboxylic acid-tert-butylester

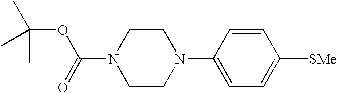

Under an inert atmosphere of argon, sodium tert-butoxide (200 mg; 2.08 mmol) was added in one portion to a room temperature stirring solution of N-Boc-piperazine (199 mg; 1.07 mmol), 1-bromo-4-(methylthio)benzene (217 mg; 1.07 mmol), 2-dicyclohexylphosphino-2′-(N,N-dimethylamino)biphenyl (113 mg; 0.29 mmol), and Pd₂(dba)₂ (57 mg; 62 μmol) in degassed 1,4-dioxan (5 mL). The mixture was sonicated for 1 minute, and stirred under microwave conditions in a sealed tube at 100° C. for 5 minutes. The crude mixture was partitioned between ethyl acetate (40 mL) and water (40 mL), and the separated aqueous extracted with ethyl acetate (40 mL). The combined organic phase was dried (MgSO₄), and concentrated in vacuo. The resulting brown oil was purified by column chromatography, giving the title compound 4-[4-(methylthio)phenyl]piperazine-1-carboxylic acid-tert-butylester as a yellow solid (259 mg; 79%). LC/MS (Ammonium bicarbonate ES+) Found 209 (M−Boc+H, T_{R} 3.47 min).

### DESCRIPTION 69

### 1-[4-(Methylthio)phenyl]piperazine

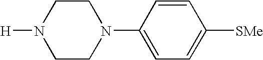

Trifluoroacetic acid (200 μL; 2.69 mmol) was added in one portion to a room temperature stirring solution of carbamate 4-[4-(methylthio)phenyl]piperazine-1-carboxylic acid-tert-butylester (78.7 mg; 0.26 mmol) in dichloromethane (1 mL). The reaction was stirred for 15 hours and purified using an SCX ion exchange column, giving 1-[4-(methylthio)phenyl]piperazine as a pale yellow solid (42.9 mg; 79%).

### DESCRIPTION 70

### 4-[4-(Methylsulfinyl)phenyl]piperazine-1-carboxylic acid-tert-butylester

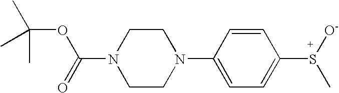

meta-Chloroperoxybenzoic acid (77%; 191 mg; 0.85 mmol) was added in one portion to a cool (0° C.) stirring solution of 4-[4-(methylthio)phenyl]piperazine-1-carboxylic acid-tert-butylester (179 mg; 0.58 mmol) in dichloromethane (3 mL). The mixture was stirred at 0° C. for 1% hours, quenched with saturated aqueous sodium hydrogen carbonate (5 mL) and diluted with water (1 mL) and dichloromethane (2 mL). The separated aqueous phase was extracted with dichloromethane (10 mL), and the combined organic phase dried (MgSO₄). Concentration in vacuo gave a yellow solid (189 mg) which was purified by column chromatography giving 4-[4-(methylsulfinyl)phenyl]piperazine-1-carboxylic acid-tert-butylester (141 mg; 75%) as a pale yellow solid. LC/MS (Ammonium bicarbonate ES+)

Found 347 (M+Na, T_{R} 2.58 min).

### DESCRIPTION 71

### 4-[4-(methylsulfinyl)phenyl]piperazine-1-carboxylic acid-tert-butylester

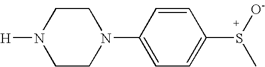

Trifluoroacetic acid (250 μL; 3.37 mmol) was added in one portion to a room temperature stirring solution of 4-[4-(methylsulfinyl)phenyl]piperazine-1-carboxylic acid-tert-butylester (141 mg; 0.436 mmol) in dichloromethane (1.7 mL). The reaction was stirred for 18 hours and purified using an SCX ion exchange column, giving 4-[4-(methylsulfinyl)phenyl]piperazine-1-carboxylic acid-tert-butylester as a white solid (92.9 mg; 93%). LC/MS (Ammonium bicarbonate ES+) Found 225 (M+H, T_{R} 1.26 min).

### DESCRIPTION 72

### 1,1-Dimethylethyl 4-(1H-pyrrolo[2,3-b]pyridin-4-yl)-1-piperazinecarboxylate

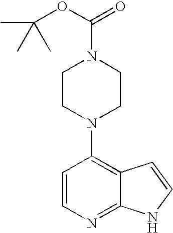

A solution of 4-bromo-1H-pyrrolo[2,3-b]pyridine (6.1 g) and N-butyloxycarbonylpiperazine (29.8 g) in N-methylpyrrolidine (20 ml) was heated on an oil bath at 135° under argon for 3 days. The mixture was dissolved in DCM (500 ml) and washed with water (6×250 ml), dried and concentrated under reduced pressure. Treatment with ether (200 ml) afforded a cream solid (7.1 g).

### DESCRIPTION 73

### 1-(Phenylsulfonyl)-4-(1-piperazinyl)-1H-pyrrolo[2,3-b]pyridine

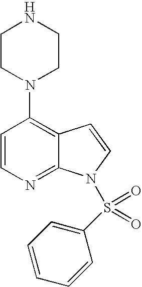

A solution of 1,1-dimethylethyl 4-(1H-pyrrolo[2,3-b]pyridin-4-yl)-1-piperazinecarboxylate (100 mg) in THF (1 ml) and DMF (1 ml) was treated with BEMP (0.19 ml). A solution of phenylsulphonyl chloride (76 mg) in THF (1 ml) was then added and the solution stirred under argon for 2.5 hours. After dilution with ethyl acetate, the reaction mixture was washed with 2M HCl and brine, dried and concentrated to a yellow oil. This was chromatographed on silica gel eluting with 10-50% ethyl acetate in pentane to give an oil (34 mg). This was treated with trifluoroacetic acid for 1 hour and the volatile components removed under reduced pressure to give the desired product as an oil (30 mg).

### DESCRIPTION 74

### 1-(Phenylsulfonyl)-4-(1-piperazinyl)-1H-indole

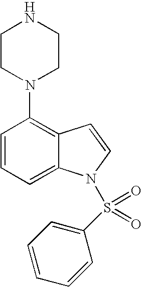

Prepared as described for 1-(phenylsulfonyl)-4-(1-piperazinyl)-1H-pyrrolo[2,3-b]pyridine.

### DESCRIPTION 75

### 1-(2-Furanylsulfonyl)-4-(1-piperazinyl)-1H-indole

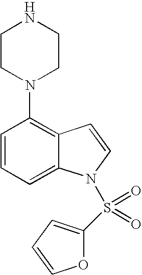

Prepared as described for 1-(phenylsulfonyl)-4-(1-piperazinyl)-1H-pyrrolo[2,3-b]pyridine.

### DESCRIPTION 76

### 1-(2-Furanylsulfonyl)-4-(1-piperazinyl)-1H-pyrrolo[2,3-b]pyridine

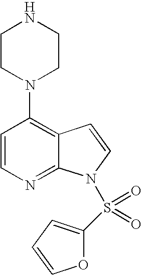

Prepared as described for 1-(phenylsulfonyl)-4-(1-piperazinyl)-1H-pyrrolo[2,3-b]pyridine.

### DESCRIPTION 77

### 1-(Methylsulfonyl)-4-(1-piperazinyl)-1H-pyrrolo[2,3-b]pyridine

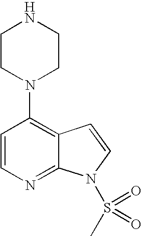

Prepared as described for 1-(phenylsulfonyl)-4-(1-piperazinyl)-1H-pyrrolo[2,3-b]pyridine.

### DESCRIPTION 78

### 4-(4-{[2-chloro-5-(methylsulfonyl)phenyl]carbonyl}-1-piperazinyl)-1-(2-furanylsulfonyl)-1H-pyrrolo[2,3-b]pyridine

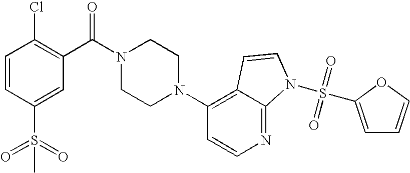

The title compound was prepared by the procedure described in Example 14, using the appropriate benzoic acid derivative and piperazine. LC/MS (ESI) Found 549 (M+1).

### DESCRIPTION 79

### 1,1-Dimethylethyl 4-(3,5-difluorophenyl)-1-piperazinecarboxylate

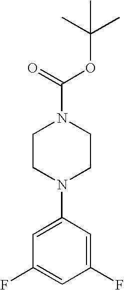

The title compound was prepared from 1-bromo-3,5-difluoro-benzene and 1-BOC-piperazine according to the procedure described in Description 61, (96.7% yield).

LC/MS (ESI) Found 243.2

### DESCRIPTION 80

### 1-(3,5-Difluorophenyl)piperazine

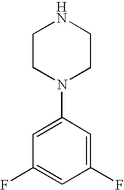

The title compound was prepared according to the procedure described in Description 62, (89% yield).

LCMS (ESI) Found 199.2 (M+1)

### DESCRIPTION 81

### 1-(3,5-Difluorophenyl)-4-{[4′-fluoro-4-(methylsulfonyl)-2-biphenylyl]carbonyl}piperazine

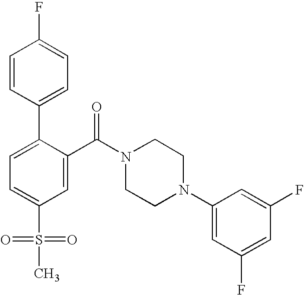

The title compound was prepared form 4′-fluoro-4-(methylsulfonyl)-2-biphenylcarboxylic acid and 1-(3,5-difluorophenyl)piperazine according to the procedure described in Example 112, (79% yield).

LC/MS (ESI) Found 475.2 (M+1).

### DESCRIPTION 82

### 1-(3,5-Difluorophenyl)-4-{[2′-fluoro-4-(methylsulfonyl)-2-biphenylyl]carbonyl}piperazine

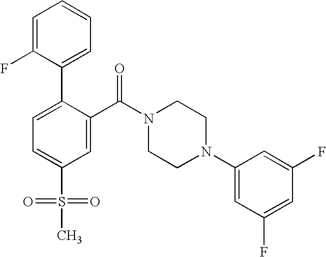

The title compound was prepared form 2′-fluoro-4-(methylsulfonyl)-2-biphenylcarboxylic acid and 1-(3,5-difluorophenyl)piperazine according to the procedure described in Example 112, (66% yield).

LC/MS (ESI) Found 475.4 (M+1).

### DESCRIPTION 83

### 2,6-Difluoro-4-(4-{[4′-fluoro-4-(methylsulfonyl)-2-biphenylyl]carbonyl}-1-piperazinyl)benzaldehyde

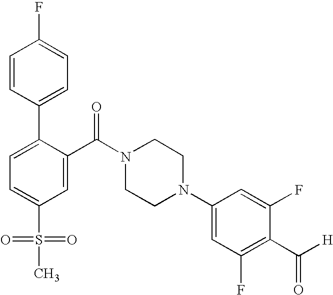

The title compound was prepared from 1-(3,5-difluorophenyl)-4-{[4′-fluoro-4-(methylsulfonyl)-2-biphenylyl]carbonyl}piperazine by the method described in Example 28 Route A, (51.5% yield).

LC/MS (ESI) Found 503.3. (M+1).

### DESCRIPTION 84

### 2,6-Difluoro-4-(4-{[2′-fluoro-4-(methylsulfonyl)-2-biphenylyl]carbonyl}-1-piperazinyl)benzaldehyde

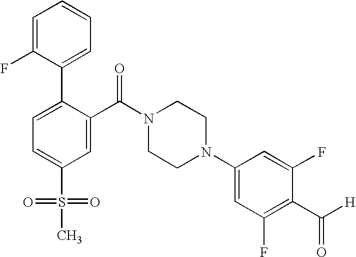

The title compound was prepared from 1-(3,5-difluorophenyl)-4-{[2′-fluoro-4-(methylsulfonyl)-2-biphenylyl]carbonyl}piperazine by the method described in Example 28 Route A, (65% yield). LC/MS (ESI) Found 503.4. (M+1).

### DESCRIPTION 85

### [2,6-Difluoro-4-(4-{[4′-fluoro-4-(methylsulfonyl)-2-biphenylyl]carbonyl}-1-piperazinyl)phenyl]methanol

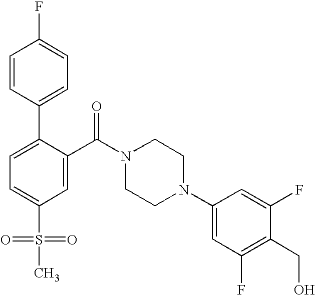

The title compound was prepared from 2,6-difluoro-4-(4-{[4′-fluoro-4-(methylsulfonyl)-2-biphenylyl]carbonyl}-1-piperazinyl)benzaldehyde by the method described in Example 15, (88% yield).

LC/MS (ESI) Found 487.2. (M+1−18).

### DESCRIPTION 86

### [2,6-Difluoro-4-(4-{[2′-fluoro-4-(methylsulfonyl)-2-biphenylyl]carbonyl}-1-piperazinyl)phenyl]methanol

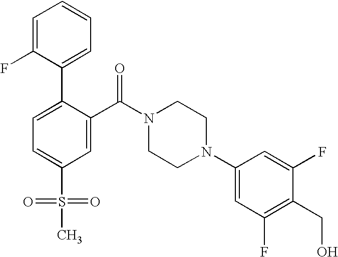

The title compound was prepared from 2,6-difluoro-4-(4-{[2′-fluoro-4-(methylsulfonyl)-2-biphenylyl]carbonyl}-1-piperazinyl)benzaldehyde by the method described in Example 15, (97% yield).

LC/MS (ESI) Found 487.4. (M+1−18). 527.3 (M+Na)

### EXAMPLE 1

### (3′-Chloro-4-methanesulfonyl-biphenyl-2-yl)-[4-(3,4-dichloro-phenyl)-piperazin-1-yl]-methanone

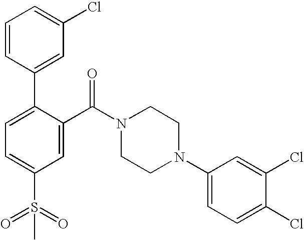

A reaction mixture of (2-chloro-5-methanesulfonyl-phenyl)-[4-(3,4-dichloro-phenyl)-piperazin-1-yl]-methanone (65 mg, 0.145 mmol), sodium bicarbonate (49 mg, 0.58 mmol), 3-chlorophenylboronic acid (45 mg, 0.29 mmol), palladium(II) acetate (3.3 mg, 14.5 μmol) and 2-(dicyclohexylphosphino)biphenyl (13 mg, 36.5 μmol) in a degassed mixture of 5:2 N,N-dimethylformamide/water (1.5 ml) under nitrogen atmosphere, was heated to 100° C. for 18 hrs. The cooled reaction mixture was filtered through a pad of silica and the cake was washed with dichloromethane. The filtrate was then concentrated in vacuo to give an oil, which was then re-dissolved in dichloromethane and washed with water. The organic layer was dried over anhydrous magnesium sulfate, filtered and concentrated in vacuo to give the crude product. Purification by flash chromatography (SiO₂) gave the pure title compound as a white solid;

d_{H} (400 MHz, CDCl₃) 8.00 (1H, dd, ArH), 7.99 (1H, d, ArH), 7.58 (1H, d, ArH), 7.44 (1H, s, ArH), 7.24 (2H, m, ArH), 7.19 (2H, m, ArH), 6.75 (1H, d, ArH), 6.52 (1H, dd, ArH), 3.76 (1H, m) 3.61 (1H, m), 3.08 (2H, m), 3.05 (3H, s, SO₂Me), 2.75 (3H, m) and 2.05 (1H, m).

LC/MS (Ammonium bicarbonate APCI) Found 523/525/527 (M+1, T_{R} 2.48 min)

The following examples were prepared by the above method in Description 22.

### EXAMPLE 2

### [4-(3,4-Dichloro-phenyl)-piperazin-1-yl]-(4-methanesulfonyl-biphenyl-2-yl)-methanone

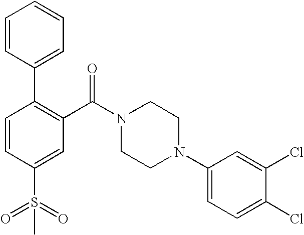

The title compound was prepared by the method described in Description 22 and was isolated as a white solid.

d_{H} (400 MHz, CDCl₃) 8.04 (1H, dd, ArH), 8.02 (1H, d, ArH), 7.66 (1H, d, ArH), 7.55-7.40 (5H, m, ArH), 7.23 (1H, d, ArH), 6.77 (1H, d, ArH), 6.56 (1H, dd, ArH), 3.84 (1H, m), 3.60 (1H, ddd), 3.16-3.05 (2H, m), 3.14 (3H, s, SO₂Me), 2.82-2.63 (3H, m) and 1.89 (1H, m).

LC/MS (Ammonium bicarbonate ESI) Found 530/532 (M+MeCN+1, T_{R} 2.49 min)

### EXAMPLE 4

### [4-(3,4-Dichloro-phenyl)-piperazin-1-yl]-(4-methanesulfonyl-2′-methoxy-biphenyl-2-yl)-methanone

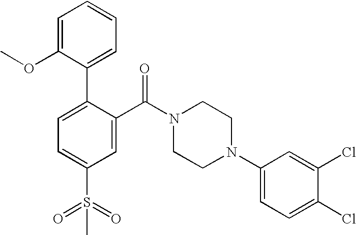

The title compound was prepared by the method described in Description 22 and was isolated as a white solid.

d_{H} (400 MHz, CDCl₃) 8.02 (2H, m, ArH), 7.62 (1H, d, ArH), 7.45 (2H, d, ArH), 7.22 (1H, dd, ArH), 6.99 (2H, d, ArH), 6.78 (1H, d, ArH), 6.60 (1H, dd, ArH), 3.79 (3H, s OMe), 3.68 (1H, m), 3.61 (1H, m), 3.17 (1H, m), 3.12 (3H, s, SO₂Me), 3.10 (1H, m), 2.93 (1H, m), 2.85 (1H, m), 2.69 (1H, m) and 2.17 (1H, m).

LC/MS (Ammonium bicarbonate ESI) Found 519/521 (M+1, T_{R} 2.45 min)

### EXAMPLE 6

### [4-(3,4-Dichloro-phenyl)-piperazin-1-yl]-(2-furan-3-yl-5-methanesulfonyl-phenyl)-methanone

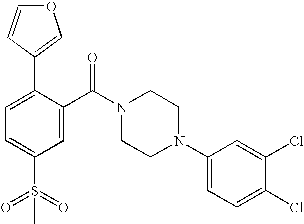

The title compound was prepared by the method described in Description 22 and was isolated as a yellow solid.

d_{H} (400 MHz, CDCl₃) 7.99 (1H, dd, ArH), 7.92 (1H, d, ArH), 7.76 (1H, br s, ArH), 7.66 (1H, d, ArH), 7.51 (1H, t, ArH), 7.28 (1H, d, ArH), 6.87 (1H, d, ArH), 6.67 (1H, br s, ArH), 6.65 (1H, dd), 3.96 (1H, m), 3.82 (1H, m), 3.22 (2H, m), 3.09 (3H, s, SO₂Me), 3.06 (2H, m), 2.95 (1H, m) and 2.48 (1H, ddd).

LC/MS (Ammonium bicarbonate ESI) Found 477/479 (M−1, T_{R} 2.33 min)

### EXAMPLE 7

### (4′-Chloro-4-methanesulfonyl-biphenyl-2-yl)-[4-(3,4-dichloro-phenyl)-piperazin-1-yl]-methanone

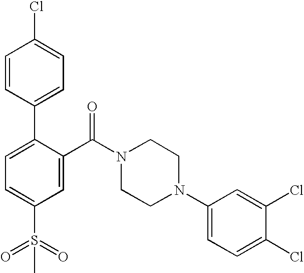

The title compound was prepared by the method described in Description 22 and was isolated as a white solid.

d_{H} (400 MHz, CDCl₃) 8.05 (1H, dd, ArH), 8.02 (1H, d, ArH), 7.65 (1H, d, ArH), 7.45 (4H, s, ArH), 7.25 (1H, dd, ArH), 6.82 (1H, d, ArH), 6.60 (1H, dd, ArH), 3.77 (1H, m), 3.69 (1H, m), 3.15-3.00 (2H, m), 3.12 (3H, s, SO₂Me), 2.89 (1H, m), 2.79 (2H, m) and 2.22 (1H, m).

LC/MS (Ammonium bicarbonate ESI) Found 564/566/568 (M+MeCN+1, T_{R} 2.62 min)

### EXAMPLE 8

### [4-(3,4-Dichloro-phenyl)-piperazin-1-yl]-(5-methanesulfonyl-2-pyridin-4-yl-phenyl)-methanone

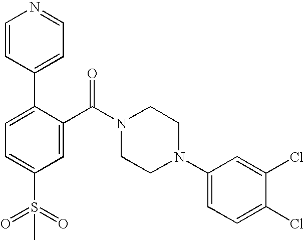

The title compound was prepared by the method described in Description 22 and was isolated as an off-white solid.

d_{H} (400 MHz, CDCl₃) 8.73 (2H, d, ArH), 8.11 (1H, dd), 8.05 (1H, d, ArH), 7.68 (1H, d, ArH), 7.43 (2H, d, ArH), 7.24 (1H, d, ArH), 6.82 (1H, d, ArH), 6.60 (1H, dd, ArH), 3.80 (1H, m), 3.69 (1H, m), 3.13 (5H, m, incl SO₂Me), 2.84 (3H, m) and 2.30 (1H, m).

LC/MS (Ammonium bicarbonate ESI) Found 490/492 (M+1, T_{R} 2.01 min)

### EXAMPLE 10

### [4-(3,4-Dichloro-phenyl)-piperazin-1-yl]-(5-methanesulfonyl-2-pyridin-3-yl-phenyl)-methanone

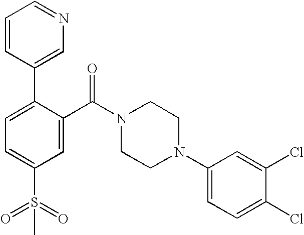

The title compound was prepared by the method described in Description 22 and was isolated as a yellow solid.

d_{H} (400 MHz, CDCl₃) 8.75 (1H, d, ArH), 8.68 (1H, dd, ArH), 8.10 (1H, dd, ArH), 8.05 (1H, s, ArH), 7.87 (1H, dd, ArH), 7.68 (1H, d, ArH), 7.40 (1H, dd, ArH), 7.25 (1H, d, ArH), 6.81 (1H, d, ArH), 6.59 (1H, dd, ArH), 3.74 (2H, m), 3.13 (3H, s, SO₂Me), 3.17 (2H, m), 2.83 (3H, m) and 2.18 (1H, m).

LC/MS (Ammonium bicarbonate APCI) Found 490 (M+1, T_{R} 1.92 min)

### EXAMPLE 12

### 2′-[4-(4-Methoxy-3,5-dichloro-phenyl)-piperazine-1-carbonyl]-4′-methanesulfonyl-biphenyl

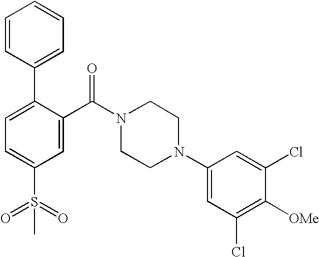

The title compound was prepared by the method described in Description 22 and was isolated as a white solid (82%).

d_{H} (400 MHz, CDCl₃) 8.05 (1H, dd, ArH), 8.03 (1H, d, ArH), 7.66 (1H, d, ArH), 7.55-7.43 (5H, m, ArH), 6.63 (2H, s, ArH), 3.84 (1H, m), 3.81 (3H, s, OMe), 3.55 (1H, ddd), 3.13 (3H, s, SO₂Me), 3.07 (2H, m), 2.70 (3H, m) and 1.85 (1H, m).

LC/MS (Ammonium bicarbonate APCI) Found 519/521 (M+1, T_{R} 2.31 min)

### EXAMPLE 13

### [4-(3,4-Dichloro-phenyl)-piperazin-1-yl]-(4′-fluoro-4-methanesulfonyl-biphenyl-2-yl)-methanone

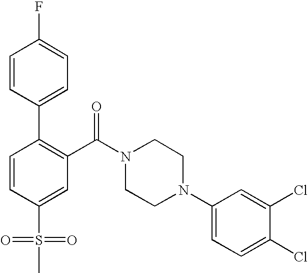

The title compound was prepared by the method described in Description 22.

d_{H} (400 MHz, CDCl₃) 8.05 (1H, dd), 8.02 (1H, d), 7.62 (1H, d), 7.50 (2H, m), 7.24 (1H, d), 7.17 (2H, t), 6.81 (1H, d), 6.60 (1H, dd), 3.73 (2H, m), 3.12 (3H, s), 3.10 (2H, m), 2.82 (3H, m), 2.20 (1H, m)

LC/MS (Ammonium bicarbonate APCI) Found 507/509 (M+1, T_{R} 2.40 min)

### EXAMPLE 14

### 1-[3-Fluoro-4-(4-{[4′-fluoro-4-(methylsulfonyl)-2-biphenylyl]carbonyl}-1-piperazinyl)phenyl]ethanone

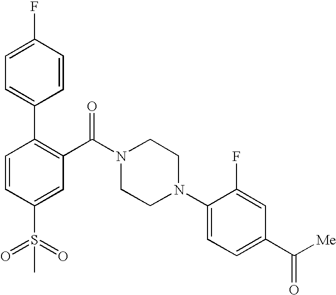

4′-Fluoro-4-(methylsulfonyl)-2-biphenylcarboxylic acid (6.5 g, 0.022 mol) and 1-[3-fluoro-4-(1-piperazinyl)phenyl]ethanone (7.34 g, 0.033 mol) were dissolved in MDC (50 ml) and stirred at room temperature for 16 hours, under an argon atmosphere, with EDC (6.5 g, 0.033 mol) and HOBt (5.1 g, 0.033 mol). The reaction solution was washed with saturated sodium hydrogen carbonate solution and dried through a Phase Sep column. The organics were evaporated to dryness and split into 3 batches for chromatography (eluent 0-80% EtOAc/pentane) to afford the title compound as a combined 9.51 g, 87%.

LC/MS (ESI) Found 499 (M+1), 521 (M+23).

### EXAMPLE 15

### 1-[3-Fluoro-4-(4-{[4′-fluoro-4-(methylsulfonyl)-2-biphenylyl]carbonyl}-1-piperazinyl)phenyl]ethanol

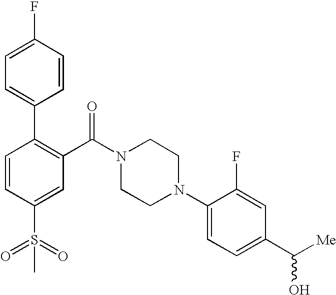

1-[3-Fluoro-4-(4-{[4′-fluoro-4-(methylsulfonyl)-2-biphenylyl]carbonyl}-1-piperazinyl)phenyl]ethanone (207 mg) was dissolved in methanol (10 ml) and sodium borohydride (17 mg) was added to the stirred solution. Stirring was continued at room temperature for 2 h. The reaction solution was then partitioned between ethyl acetate and water. The organic phase was washed with brine, dried (MgSO4) and evaporated to yield the title compound as a white foam, (201 mg, 98% yield), LC/MS (ESI) Found 523 (M+23).

### EXAMPLE 16

### (−)-1-[3-Fluoro-4-(4-{[4′-fluoro-4-(methylsulfonyl)-2-biphenylyl]carbonyl}-1-piperazinyl)phenyl]ethanol (Faster running enantiomer)

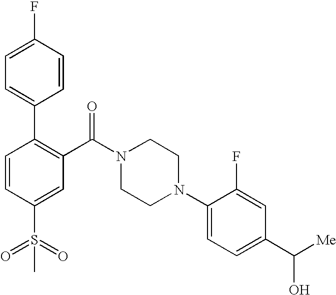

To a stirred solution of (R)-2-methyl-CBS-oxazaborolidine (10.5 ml, 1M in toluene) in anhydrous tetrahydrofuran (40 ml) at 0° C. under argon was added borane-methyl sulphide complex (10.5 ml, 2M in tetrahydrofuran) dropwise over 2 mins. After a further 5 minutes a solution of 1-[3-fluoro-4-(4-{[4′-fluoro-4-(methylsulfonyl)-2-biphenylyl]carbonyl}-1-piperazinyl)phenyl]ethanone (4.75 g, 9.53 mmol) in tetrahydrofuran (140 ml) was added dropwise over 30 mins. The reaction mixture was stirred at 0° C. for 1 h, methanol (10 ml) added slowly and the mixture partitioned between ethyl acetate and saturated ammonium chloride. The organic layer was dried (Na₂SO₄) and evaporated in vacuo. The residue was triturated with DCM, filtered and the filtrate evaporated in vacuo. Chromatography on silica gel eluting with 0-100% ethyl acetate in pentane gradient afforded the title compound as a colourless solid (3.6 g, 75%). LC/MS (ESI) Found 501 (M+1). [α]_{D}=−15.96° (30.2° C., c=1.01, CHCl₃). ee=99.0% (Chiralcel OJ (250 mm×4.6 mm i.d; 10 micron particle size) as the stationary phase with a mobile phase of Hexane Fraction: Absolute Ethanol (50:50) v/v; pump-mixed) at a flow-rate of 1.0 mL/min; UV 215 nM).

### EXAMPLE 17

### (+)-1-[3-Fluoro-4-(4-{[4′-fluoro-4-(methylsulfonyl)-2-biphenylyl]carbonyl}-1-piperazinyl)phenyl]ethanol (Slower running enantiomer)

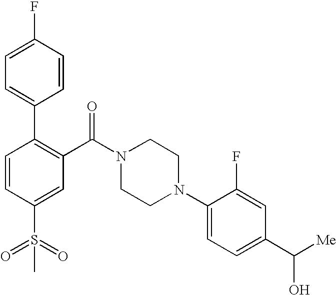

The title compound was prepared from 1-[3-fluoro-4-(4-{[4′-fluoro-4-(methylsulfonyl)-2-biphenylyl]carbonyl}-1-piperazinyl)phenyl]ethanone (4.75 g) in an identical method to that outlined in Example 16 except that (S)-2-methyl-CBS-oxazaborolidine (10.5 ml, 1M in toluene) was used as the chiral auxiliary. (3.4 g, 71%). LC/MS (ESI) Found 501 (M+1). [α]_{D}=+15.56° (28.7° C., c=1.06, CHCl₃). ee=99.0% (Chiralcel OJ (250 mm×4.6 mm i.d; 10 micron particle size) as the stationary phase with a mobile phase of Hexane Fraction: Absolute Ethanol (50:50) v/v; pump-mixed) at a flow-rate of 1.0 mL/min; UV 215 nM).

### EXAMPLE 18

### 1-{2-Fluoro-4-[1-(methyloxy)ethyl]phenyl}-4-{[4′-fluoro-4-(methylsulfonyl)-2-biphenylyl]carbonyl}piperazine

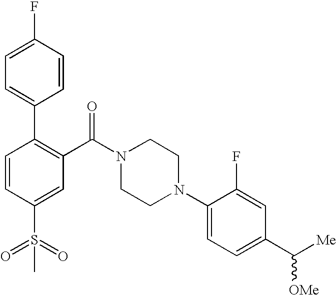

Route A.

1-[3-Fluoro-4-(4-{[4′-fluoro-4-(methylsulfonyl)-2-biphenylyl]carbonyl}-1-piperazinyl)phenyl]ethanol (0.195 g) was dissolved in N,N-dimethylformamide (10 ml) and stirred at room temperature under argon. Sodium hydride (60% dispersion in oil, 20 mg) was added, followed by methyl iodide (0.033 ml). Stirring was continued at room temperature for 2 h. The reaction mixture was then partitioned between ethyl acetate and water. The organic solution was washed twice with water, dried (MgSO₄) and evaporated to yield the crude product, which was purified by silica gel column chromatography, eluting with a gradient of 0 to 100% ethyl acetate in hexane. The title compound was obtained as a white foam (39% yield), LC/MS (ESI) Found 537 (M+23).

Route B.

A mixture of methanol (70 ml) and toluene (100 ml) containing p-toluenesulfonic acid monohydrate (0.07 g) was heated under argon at reflux using a Dean-Stark apparatus for 30 min. 1-[3-Fluoro-4-(4-{[4′-fluoro-4-(methylsulfonyl)-2-biphenylyl]carbonyl}-1-piperazinyl)phenyl]ethanol (0.3 g) in dioxan (12 ml) was added dropwise over 10 min and heating continued for a further 40 min. The solution was cooled, saturated sodium hydrogencarbonate (4 ml) added and the mixture evaporated. The resultant was partitioned between dichloromethane and water and the organic phase separated, dried and evaporated in vacuo. Reevaporation from toluene (x3) afforded the crude product that was chromatographed over silica gel, eluting with a gradient of 0 to 70% ethyl acetate in pentane to afford the title compound as a white amorphous solid (0.274 g; 89% yield).

LC/MS (ESI) Found 537 (M+23).

### EXAMPLE 19

### (−)1-{2-Fluoro-4-[1-(methyloxy)ethyl]phenyl}-4-{[4′-fluoro-4-(methylsulfonyl)-2-biphenylyl]carbonyl}piperazine

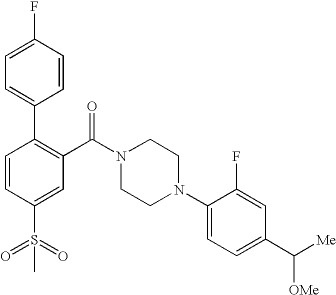

The title compound was prepared from (−)-1-[3-fluoro-4-(4-{[4′-fluoro-4-(methylsulfonyl)-2-biphenylyl]carbonyl}-1-piperazinyl)phenyl]ethanol (Faster running enantiomer; Example 16; 3.6 g, 7.19 mmol) using the method of Example 18, Route A, to give the title compound as a colourless solid (1.74 g, 47%). LC/MS (ESI) Found 537 (M+23). [α]_{D}=−41.01° (27.7° C., c=0.99, CHCl₃).

### EXAMPLE 20

### (+)1-{2-Fluoro-4-[1-(methyloxy)ethyl]phenyl}-4-{[4′-fluoro-4-(methylsulfonyl)-2-biphenylyl]carbonyl}piperazine

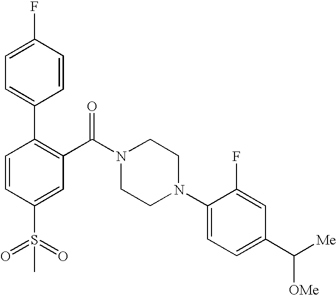

The title compound was prepared from (+)-1-[3-fluoro-4-(4-{[4′-fluoro-4-(methylsulfonyl)-2-biphenylyl]carbonyl}-1-piperazinyl)phenyl]ethanol; Slower running enantiomer; Example 17; 3.4 g) using the method of Example 18, Route A, to give the title compound as a colourless solid (1.66 g, 47%). LC/MS (ESI) Found 537 (M+23). [α]_{D}=+41.110 (27.5° C., c=0.995, CHCl₃).

### EXAMPLE 21

### 3-Fluoro-4-(4-{[4′-fluoro-4-(methylsulfonyl)-2-biphenylyl]carbonyl}-1-piperazinyl)benzaldehyde

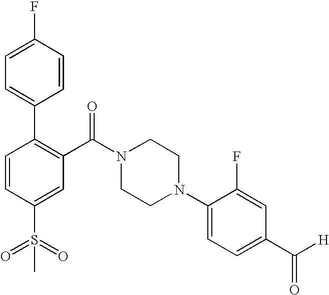

The title compound was prepared from 4′-fluoro-4-(methylsulfonyl)-2-biphenylcarboxylic acid and 3-fluoro-4-(1-piperazinyl)benzaldehyde (prepared according to the procedure in Dyes and Pigments, 59, 2003, p 163) by the procedure described in Example 14 (64% yield).

LC/MS (ESI) Found 485 (M+1).

### EXAMPLE 22

### [3-Fluoro-4-(4-{[4′-fluoro-4-(methylsulfonyl)-2-biphenylyl]carbonyl}-1-piperazinyl)phenyl]methanol

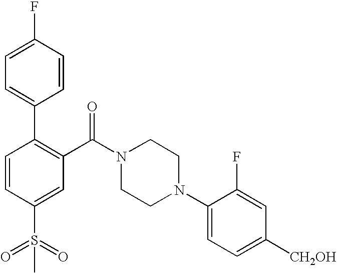

The title compound was prepared from 3-fluoro-4-(4-{[4′-fluoro-4-(methylsulfonyl)-2-biphenylyl]carbonyl}-1-piperazinyl)benzaldehyde by the procedure described in Example 15 (95% yield).

LC/MS (ESI) Found 509 (M+23).

### EXAMPLE 23

### 1-{2-Fluoro-4-[(methyloxy)methyl]phenyl}-4-{[4′-fluoro-4-(methylsulfonyl)-2-biphenylyl]carbonyl}piperazine

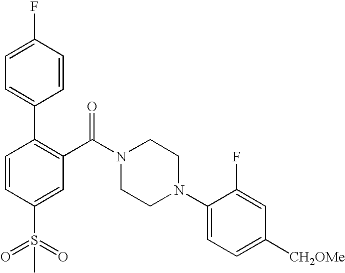

The title compound was prepared from [3-fluoro-4-(4-{[4′-fluoro-4-(methylsulfonyl)-2-biphenylyl]carbonyl}-1-piperazinyl)phenyl]methanol by the procedure described in Example 18, Route A. (53% yield).

LC/MS (ESI) Found 501 (M+1), 523 (M+23).

### EXAMPLE 24

### 1-(4-{[(Cyclopropylmethyl)oxy]methyl}-2-fluorophenyl)-4-{[4′-fluoro-4-(methylsulfonyl)-2-biphenylyl]carbonyl}piperazine

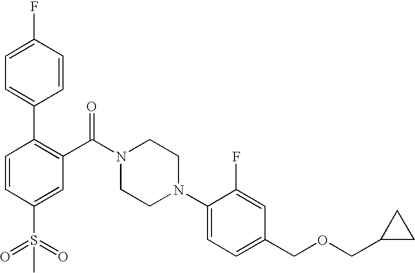

[3-Fluoro-4-(4-{[4′-fluoro-4-(methylsulfonyl)-2-biphenylyl]carbonyl}-1-piperazinyl)phenyl]methanol (120 mg), p-toluenesulfonic acid monohydrate (20 mg) and cyclopropylmethanol (0.75 ml) were dissolved in 1,4-dioxan (10 ml) and toluene (10 ml) and heated to 80° C. for 6 h with stirring under an atmosphere of argon. After cooling, the solution was evaporated and the residue partitioned between dichloromethane and saturated sodium hydrogen carbonate solution. The organic solution was washed with water and brine and dried (MgSO4). After filtration and evaporation, the residue was chromatographed over silica gel. Elution with a gradient of 0 to 100% ethyl acetate in hexane gave the title compound as a white solid (26% yield).

LC/MS (ESI) Found 563 (M+23).

### EXAMPLE 25

### 1-[3,5-Dichloro-4-(methyloxy)phenyl]-4-{[4-(methylsulfinyl)-2-biphenylyl]carbonyl}piperazine

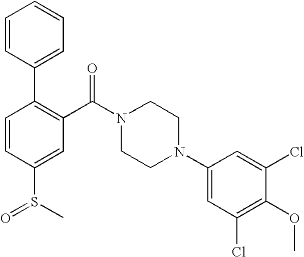

Title compound prepared as described in Example 14. Obtained as crisp foam (165 mg, 87%). LC/MS Found 503 (ESI) (M+1).

The R- and S-isomers were separated using a Chiralpak AD-H column eluting isocratically with hexane-ethanol (60:40). The faster running isomer had a retention time of 24 mins and the slower running isomer 34 mins.

### EXAMPLE 26

### 2-({4-[3,5-Dichloro-4-(methyloxy)phenyl]-1-piperazinyl}carbonyl)-N-methyl-4-biphenylsulfonamide

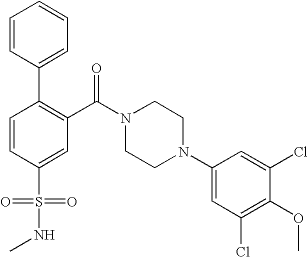

4-[(Methylamino)sulfonyl]-2-biphenylcarboxylic acid was treated as described in Example 14. Obtained title compound as crisp foam (67%). LC/MS Found 534 (ESI) (M+1).

### EXAMPLE 27

### 1-[2,6-Dichloro-4-(4-{[4′-fluoro-4-(methylsulfonyl)-2-biphenylyl]carbonyl}-1-piperazinyl)phenyl]ethanol

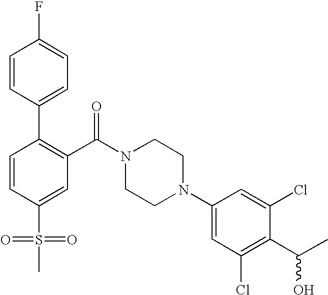

A solution of 1-[2,6-dichloro-4-(4-{[4′-fluoro-4-(methylsulfonyl)-2-biphenylyl]carbonyl}-1-piperazinyl)phenyl]ethanone (86 mg) in THF (3 ml) was treated with a 2M solution of lithium borohydride in THF (80 ul) and stirred for 1 hour under argon. The solvent was removed under reduced pressure and the residue diluted with ethyl acetate and saturated NaHCO3. The organic layer was evaporated to afford the desired product as a solid (81 mg). LC/MS Found 573 (ESI) (M+Na).

### EXAMPLE 28

### 2,6-Dichloro-4-(4-{[4′-fluoro-4-(methylsulfonyl)-2-biphenylyl]carbonyl}-1-piperazinyl)benzaldehyde

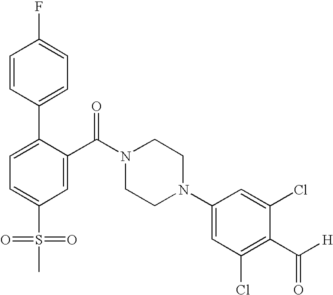

Route A.

To a stirred solution of 1-(3,5-dichlorophenyl)-4-{[4′-fluoro-4-(methylsulfonyl)-2-biphenylyl]carbonyl}piperazine (0.2 g, 0.395 mmol) in anhydrous dimethylformamide (1 ml) under argon was added phosphorous oxychloride (0.08 ml, 0.8 mmol). The reaction mixture was heated at 102° C. for 2 h, cooled, poured into 1N sodium hydroxide (40 ml) and extracted with ethyl acetate (2×40 ml). Organics were washed with water (20 ml), brine (20 ml), dried (Na₂SO₄) and evaporated in vacuo. The residue was purified by chromatography on silica gel eluting with 0-100% ethyl acetate in pentane gradient to afford the title compound as a colourless solid (0.115 g, 55%). LC/MS (ESI) Found 535 (M+1).

Route B.

The title compound was prepared from 4′-fluoro-4-(methylsulfonyl)-2-biphenylcarboxylic acid and 1-(3,5-dichloro-4-formylphenyl)piperazine by the method described in Example 14.

### EXAMPLE 29

### [2,6-Dichloro-4-(4-{[4′-fluoro-4-(methylsulfonyl)-2-biphenylyl]carbonyl}-1-piperazinyl)phenyl]methanol

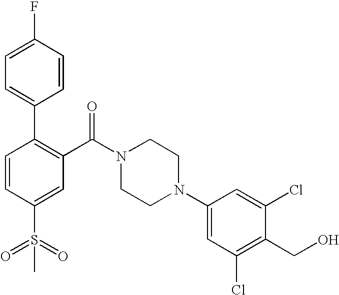

Prepared from the aldehyde of Example 28 by reduction with lithium borohydride as in Example 27, or using sodium borohydride as in Example 15.

LC/MS Found 559 (ESI) (M+Na).

The following compounds were prepared by the procedure described in Example 14, using the appropriate benzoic acid derivative and piperazine.

| Example | | | LC/MS |
|---|---|---|---|
| No. | Structure | Name | (ESI) |
| 30 | 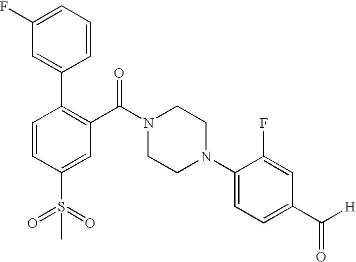 | 3-fluoro-4-(4-{[3′-fluoro-4-(methylsulfonyl)-2-biphenylyl]carbonyl}-1-piperazinyl)benzaldehyde | 485(M + 1) |
| 31 | 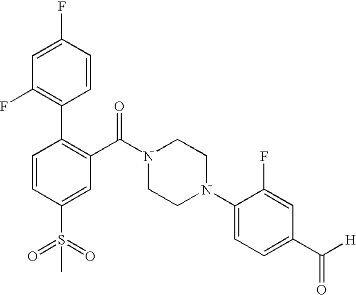 | 4-(4-{[2′,4′-difluoro-4-(methylsulfonyl)-2-biphenylyl]carbonyl}-1-piperazinyl)-3-fluorobenzaldehyde | 503(M + 1) |
| 32 | 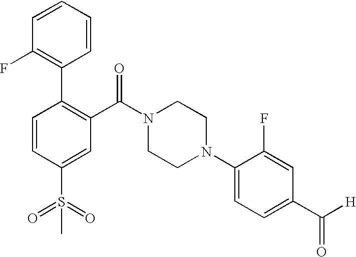 | 3-fluoro-4-(4-{[2′-fluoro-4-(methylsulfonyl)-2-biphenylyl]carbonyl}-1-piperazinyl)benzaldehyde | 485(M + 1) |
| 33 | 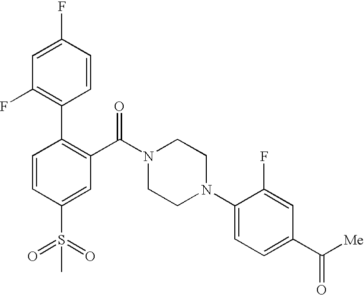 | 1-[4-(4-{[2′,4′-difluoro-4-(methylsulfonyl)-2-biphenylyl]carbonyl}-1-piperazinyl)-3-fluorophenyl]ethanone | 517(M + 1) |
| 34 | 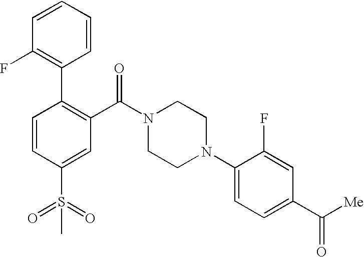 | 1-[3-fluoro-4-(4-{[2′-fluoro-4-(methylsulfonyl)-2-biphenylyl]carbonyl}-1-piperazinyl)phenyl]ethanone | 499(M + 1) |
| 35 | 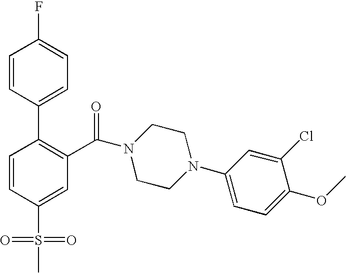 | 1-[3-chloro-4-(methyloxy)phenyl]-4-{[4′-fluoro-4-(methylsulfonyl)-2-biphenylyl]carbonyl}piperazine | 503(M + 1) |
| 36 | 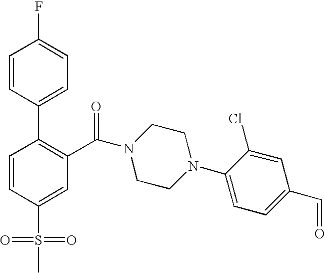 | 3-chloro-4-(4-{[4′-fluoro-4-(methylsulfonyl)-2-biphenylyl]carbonyl}-1-piperazinyl)benzaldehyde | 501(M + 1) |
| 37 | 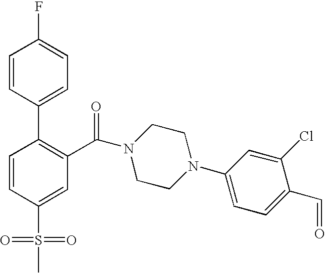 | 2-chloro-4-(4-{[4′-fluoro-4-(methylsulfonyl)-2-biphenylyl]carbonyl}-1-piperazinyl)benzaldehyde | 501(M + 1) |
| 38 | 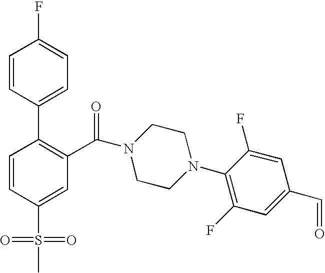 | 3,5-difluoro-4-(4-{[4′-fluoro-4-(methylsulfonyl)-2-biphenylyl]carbonyl}-1-piperazinyl)benzaldehyde | 503(M + 1) |
| 39 | 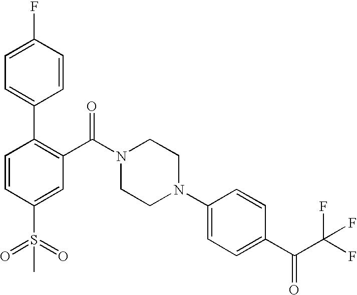 | 2,2,2-trifluoro-1-[4-(4-{[4′-fluoro-4-(methylsulfonyl)-2-biphenylyl]carbonyl}-1-piperazinyl)phenyl]ethanone | 535(M + 1) |
| 40 | 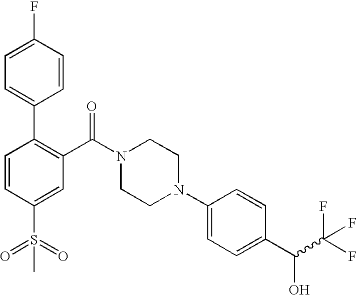 | 2,2,2-trifluoro-1-[4-(4-{[4′-fluoro-4-(methylsulfonyl)-2-biphenylyl]carbonyl}-1-piperazinyl)phenyl]ethanol | 537(M + 1) |
| 41 | 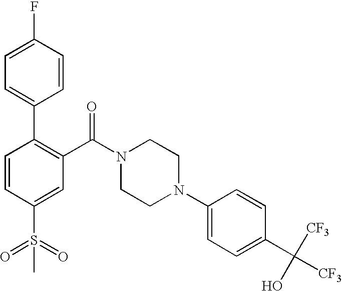 | 1,1,1,3,3,3-hexafluoro-2-[4-(4-{[4′-fluoro-4-(methylsulfonyl)-2-biphenylyl]carbonyl}-1-piperazinyl)phenyl]-2-propanol | 605(M + 1) |
| 42 | 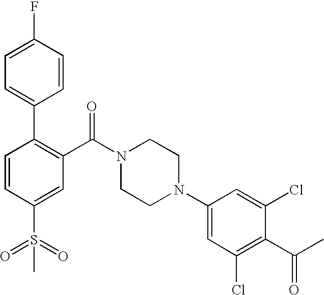 | 1-[2,6-dichloro-4-(4-{[4′-fluoro-4-(methylsulfonyl)-2-biphenylyl]carbonyl}-1-piperazinyl)phenyl]ethanone | 549(M + 1) |
| 43 | 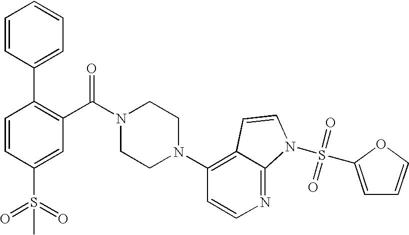 | 1-(2-furanylsulfonyl)-4-(4-{[4-(methylsulfonyl)-2-biphenylyl]carbonyl}-1-piperazinyl)-1H-pyrrolo[2,3-b]pyridine | 591(M + 1) |
| 44 | 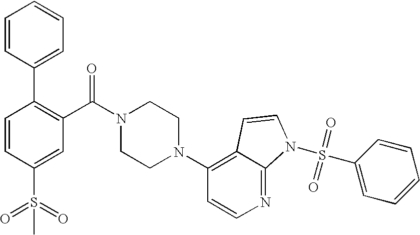 | 4-(4-{[4-(methylsulfonyl)-2-biphenylyl]carbonyl}-1-piperazinyl)-1-(phenylsulfonyl)-1H-pyrrolo[2,3-b]pyridine | 601(M + 1) |
| 45 | 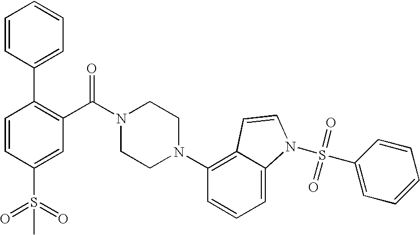 | 4-(4-{[4-(methylsulfonyl)-2-biphenylyl]carbonyl}-1-piperazinyl)-1-(phenylsulfonyl)-1H-indole | 600(M + 1) |
| 46 | 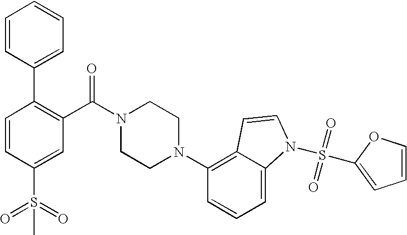 | 1-(2-furanylsulfonyl)-4-(4-{[4-(methylsulfonyl)-2-biphenylyl]carbonyl}-1-piperazinyl)-1H-indole | 590(M + 1) |
| 47 | 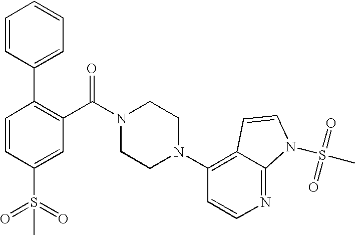 | 1(methylsulfonyl)-4-(4-{[4-(methylsulfonyl)-2-biphenylyl]carbonyl}-1-piperazinyl)-1H-pyrrolo[2,3-b]pyridine | 539(M + 1) |
| 48 | 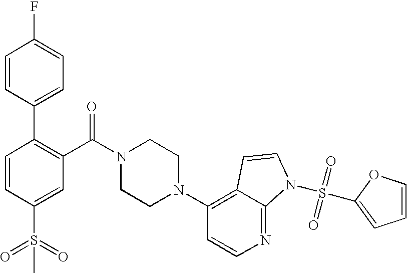 | 4-(4-{[4′-fluoro-4-(methylsulfonyl)-2-biphenylyl]carbonyl}-1-piperazinyl)-1-(2-furanylsulfonyl)-1H-pyrrolo[2,3-b]pyridine | 609(M + 1) |
| 50 | 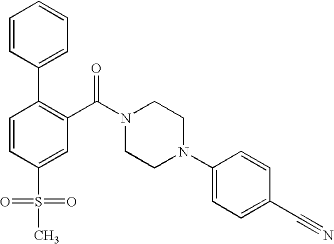 | 4-(4-{[4-(methylsulfonyl)biphenyl-2-yl]carbonyl}piperazin-1-yl)benzonitrile | 446(M + 1) |
| 51 | 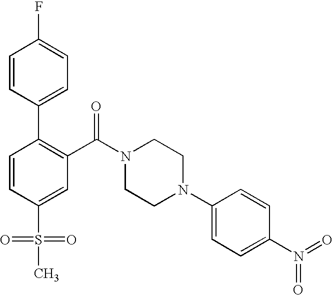 | 1-{[4′-fluoro-4-(methylsulfonyl)biphenyl-2-yl]carbonyl}-4-(4-nitrophenyl)piperazine | 484(M + 1) |
| 52 | 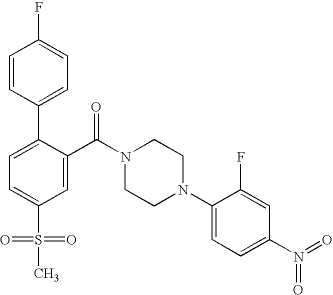 | 1-{[4′-fluoro-4-(methylsulfonyl)biphenyl-2-yl]carbonyl}-4-(2-fluoro-4-nitrophenyl)piperazine | |
| 53 | 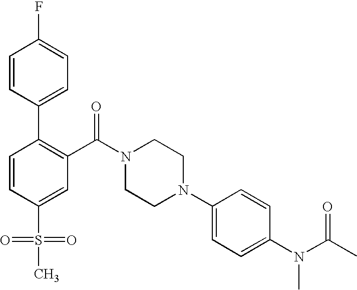 | N-[4-(4-{[4′-fluoro-4-(methylsulfonyl)-2-biphenylyl]carbonyl}-1-piperazinyl)phenyl]-N-methylacetamide | 510(M + 1) |
| 54 | 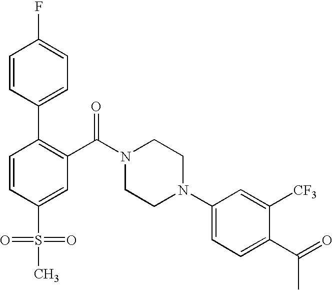 | 1-[4-(4-{[4′-fluoro-4-(methylsulfonyl)-2-biphenylyl]carbonyl}-1-piperazinyl)-2-(trifluoromethyl)phenyl]ethanone | 549(M + 1) |
| 55 | 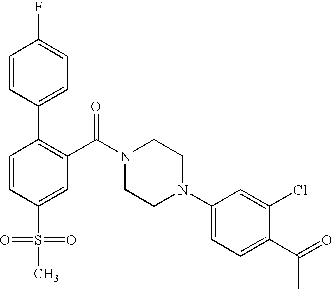 | 1-[2-chloro-4-(4-{[4′-fluoro-4-(methylsulfonyl)-2-biphenylyl]carbonyl}-1-piperazinyl)phenyl]ethanone | 515/517(M + 1) |
| 56 | 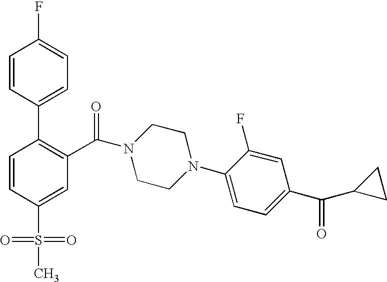 | cyclopropyl[3-fluoro-4-(4-{[4′-fluoro-4-(methylsulfonyl)-2-biphenylyl]carbonyl}-1-piperazinyl)phenyl]methanone | 525(M + 1) |

The following compounds were prepared by the procedure described in Example 15.

| | | | |
|---|---|---|---|
| Example | | | LC/MS |
| No. | Structure | Name | (ESI) |

| | | | |
|---|---|---|---|
| 57 | 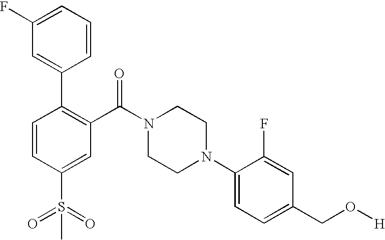 | [3-fluoro-4-(4-{[3′-fluoro-4-(methylsulfonyl)-2-biphenylyl]carbonyl}-1-piperazinyl)phenyl]methanol | 487(M + 1) |
| 58 | 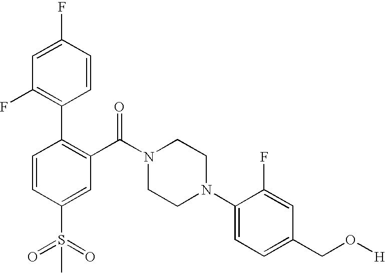 | [4-(4-{[2′,4′-difluoro-4-(methylsulfonyl)-2-biphenylyl]carbonyl}-1-piperazinyl)-3-fluorophenyl]methanol | 505(M + 1), 527(M + 23) |
| 59 | 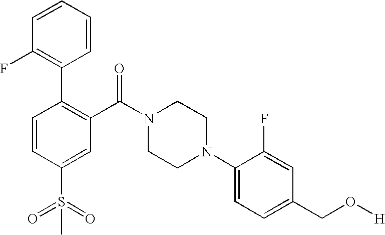 | [3-fluoro-4-(4-{[2′-fluoro-4-(methylsulfonyl)-2-biphenylyl]carbonyl}-1-piperazinyl)phenyl]methanol | 487(M + 1) |
| 60 | 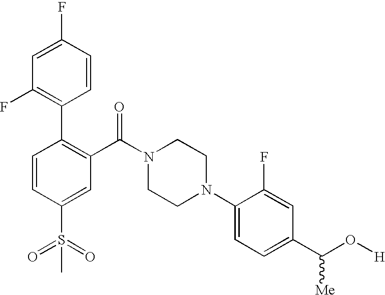 | 1-[4-(4-{[2′,4′-difluoro-4-(methylsulfonyl)-2-biphenylyl]carbonyl}-1-piperazinyl)-3-fluorophenyl]ethanol | |
| 61 | 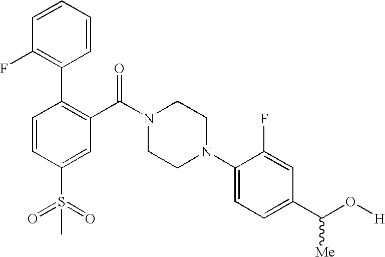 | 1-[3-fluoro-4-(4-{[2′-fluoro-4-(methylsulfonyl)-2-biphenylyl]carbonyl}-1-piperazinyl)phenyl]ethanol | 501(M + 1), 523(M + 23) |
| 62 | 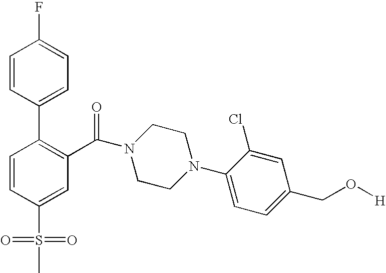 | [3-chloro-4-(4-{[4′-fluoro-4-(methylsulfonyl)-2-biphenylyl]carbonyl}-1-piperazinyl)phenyl]methanol | 503(M + 1) |
| 63 | 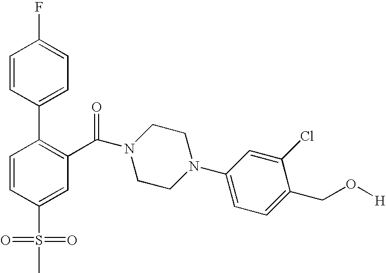 | [2-chloro-4-(4-{[4′-fluoro-4-(methylsulfonyl)-2-biphenylyl]carbonyl}-1-piperazinyl)phenyl]methanol | 525(M + 23) |
| 64 | 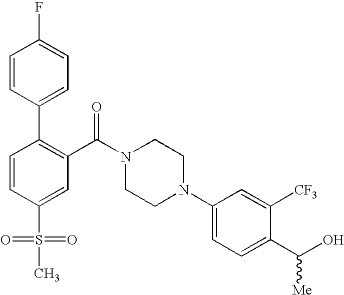 | 1-[4-(4-{[4′-fluoro-4-(methylsulfonyl)-2-biphenylyl]carbonyl}-1-piperazinyl-2-(trifluoromethyl)phenyl]ethanol | 525(M + 1) |
| 65 | 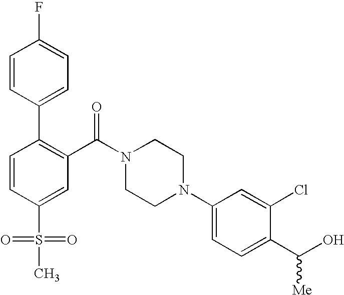 | 1-[2-chloro-4-(4-{[4′-fluoro-4-(methylsulfonyl)-2-biphenylyl]carbonyl}-1-piperazinyl)phenyl]ethanol | 517/519(M + 1) |
| 66 | 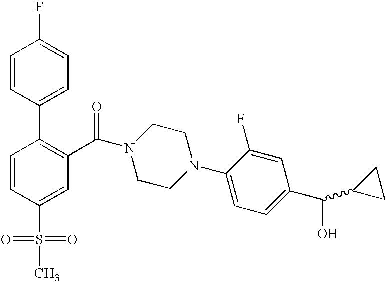 | cyclopropyl[3-fluoro-4-(4-{[4′-fluoro-4-(methylsulfonyl)-2-biphenylyl]carbonyl}-1-piperazinyl)phenyl]methanol | |

The following compounds were prepared by the procedure described in Example 18, Route A, using the appropriate alkyl halide as reagent, or Example 18, Route B, using the appropriate alcohol (as in Example 24), or Description 55 for the alkylated phenol derivatives.

| Example | | | LC/MS |
|---|---|---|---|
| No. | Structure | Name | (ESI) |
| 67 | 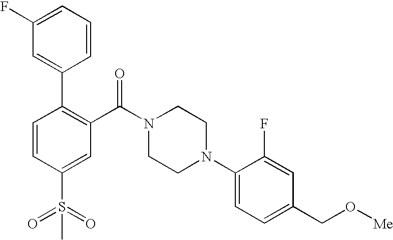 | 1-{2-fluoro-4-[(methyloxy)methyl]phenyl}-4-{[3′-fluoro-4-(methylsulfonyl)-2-biphenylyl]carbonyl}piperazine | 501(M + 1), 523(M + 23) |
| 68 | 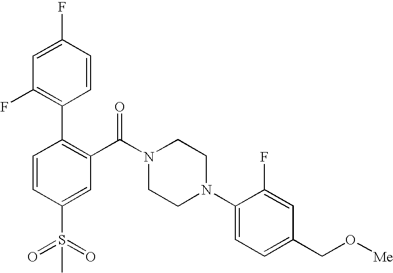 | 1-{[2′,4′-difluoro-4-(methylsulfonyl)-2-biphenylyl]carbonyl}-4-{2-fluoro-4-[(methyloxy)methyl]phenyl}piperazine | 519(M + 1) |
| 69 | 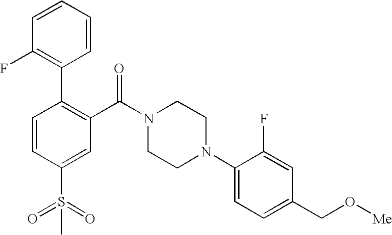 | 1-{2-fluoro-4-[(methyloxy)methyl]phenyl}-4-{[2′-fluoro-4-(methylsulfonyl)-2-biphenylyl]carbonyl}piperazine | 501(M + 1), 523(M + 23) |
| 70 | 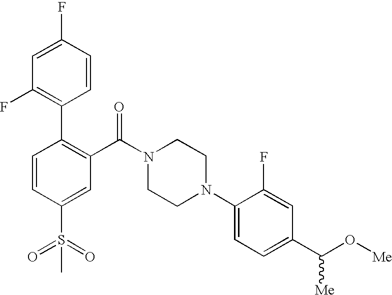 | 1-{[2′,4′-difluoro-4-(methylsulfonyl)-2-biphenylyl]carbonyl}-4-{2-fluoro-4-[1-(methyloxy)ethyl]phenyl}piperazine | 533(M + 1), 555(M + 23) |
| 71 | 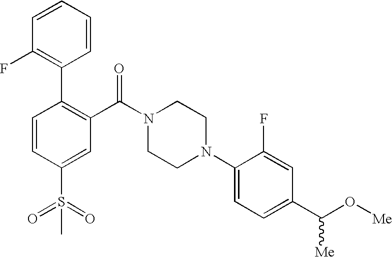 | 1-{2-fluoro-4-[1-(methyloxy)ethyl]phenyl}-4-{[2′-fluoro-4-(methylsulfonyl)-2-biphenylyl]carbonyl}piperazine | 515(M + 1), 537(M + 23) |
| 72 | 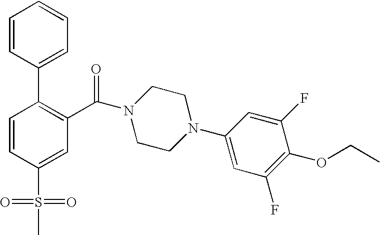 | 1-[4-(ethyloxy)-3,5-difluorophenyl]-4-{[4-(methylsulfonyl)-2-biphenylyl]carbonyl}piperazine | 501(M + 1) |
| 73 | 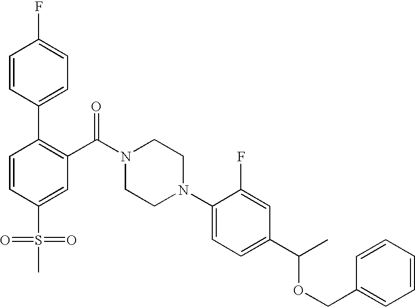 | 1-{[4′-fluoro-4-(methylsulfonyl)-2-biphenylyl]carbonyl}-4-(2-fluoro-4-{1-[(phenylmethyl)oxy]ethyl}phenyl)piperazine | 613(M + 23) |
| 74 | 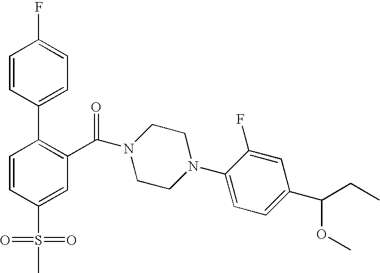 | 1-{2-fluoro-4-[1-(methyloxy)propyl]phenyl}-4-{[4′-fluoro-4-(methylsulfonyl)-2-biphenylyl]carbonyl}piperazine | 551(M + 23) |
| 75 | 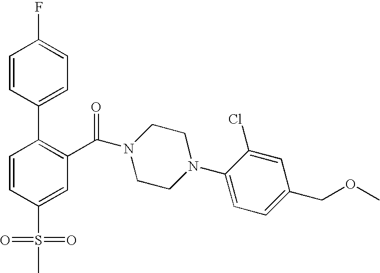 | 1-{2-chloro-4-[(methyloxy)methyl]phenyl}-4-{[4′-fluoro-4-(methylsulfonyl)-2-biphenylyl]carbonyl}piperazine | 503(M + 1) |
| 76 | 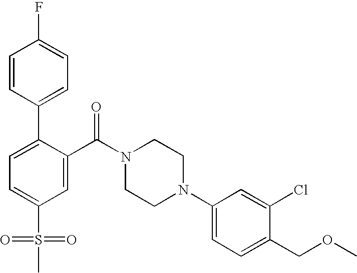 | 1-{3-chloro-4-[(methyloxy)methyl]phenyl}-4-{[4′-fluoro-4-(methylsulfonyl)-2-biphenylyl]carbonyl}piperazine | 525(M + 23) |
| 77 | 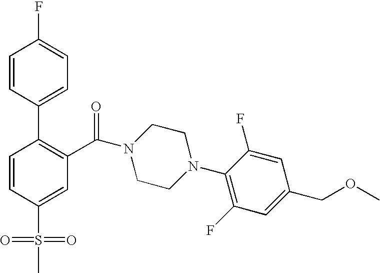 | 1-{2,6-difluoro-4-[(methyloxy)methyl]phenyl}-4-{[4′-fluoro-4-(methylsulfonyl)-2-biphenylyl]carbonyl}piperazine | 505(M + 1) |
| 78 | 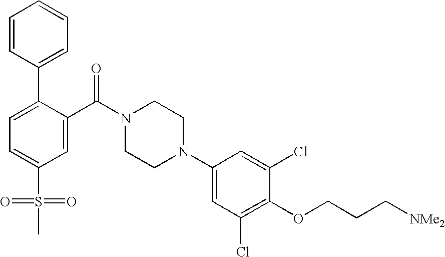 | (3-{[2,6-dichloro-4-(4-{[4-(methylsulfonyl)-2-biphenylyl]carbonyl}-1-piperazinyl)phenyl]oxy}propyl)dimethylamine | 590(M + 1) |
| 79 | 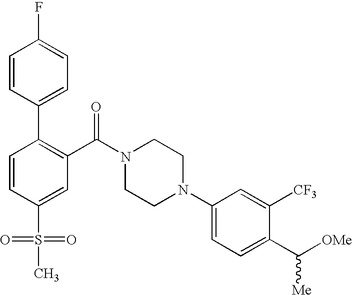 | 1-{[4′-fluoro-4-(methylsulfonyl)-2-biphenylyl]carbonyl}-4-[4-[1-(methyloxy)ethyl]-3-(trifluoromethyl)phenyl]piperazine | 587(M + 23) |
| 80 | 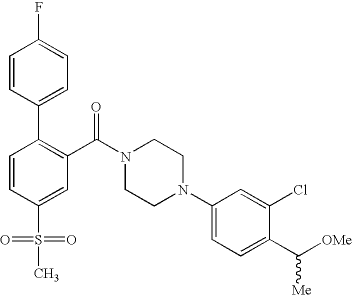 | 1-{3-chloro-4-[1-(methyloxy)ethyl]phenyl}-4-{[4′-fluoro-4-(methylsulfonyl)-2-biphenylyl]carbonyl}piperazine | 553/555(M + 23) |
| 81 | 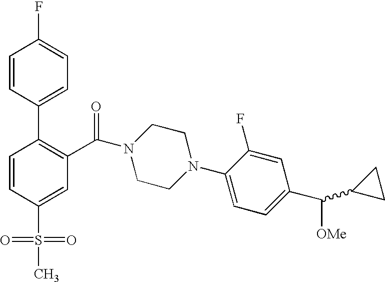 | 1-{4-[cyclopropyl(methyloxy)methyl]-2-fluorophenyl}-4-{[4′-fluoro-4-(methylsulfonyl)-2-biphenylyl]carbonyl}piperazine | 563(M + 23) |

The following compounds were prepared by the procedure described in Example 24, using the appropriate alcohol as reagent.

| Example | | | LC/MS |
|---|---|---|---|
| No. | Structure | Name | (ESI) |
| 82 | 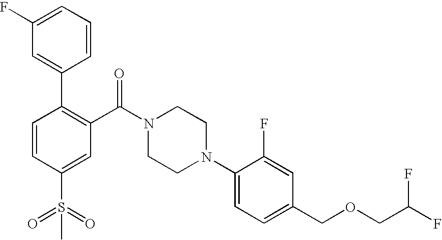 | 1-(4-{[(2,2-difluoroethyl)oxy]methyl}-2-fluorophenyl)-4-{[3′-fluoro-4-(methylsulfonyl)-2-biphenylyl]carbonyl}piperazine | 551(M + 1), 573(M + 23) |
| 83 | 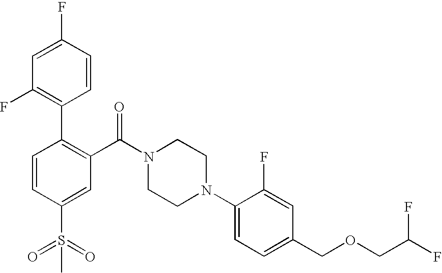 | 1-(4-{[(2,2-difluoroethyl)oxy]methyl}-2-fluorophenyl)-4-{[2′,4′-difluoro-4-(methylsulfonyl)-2-biphenylyl]carbonyl}piperazine | 569(M + 1), 591(M + 23) |
| 84 | 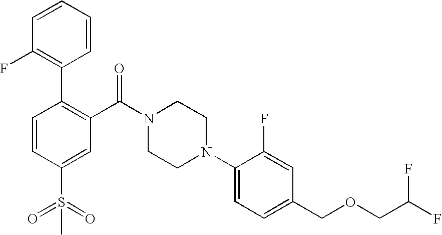 | 1-(4-{[(2,2-difluoroethyl)oxy]methyl}-2-fluorophenyl)-4-{[2′-fluoro-4-(methylsulfonyl)-2-biphenylyl]carbonyl}piperazine | 551(M + 1), 573(M + 23) |
| 85 | 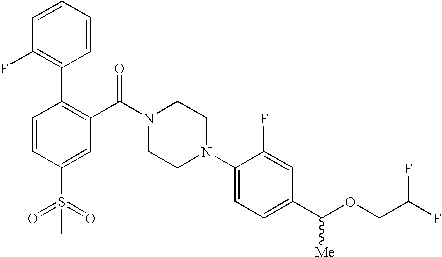 | 1-(4-{1-[(2,2-difluoroethyl)oxy]ethyl}-2-fluorophenyl)-4-{[2′-fluoro-4-(methylsulfonyl)-2-biphenylyl]carbonyl}piperazine | 565(M + 1), 587(M + 23) |
| 86 | 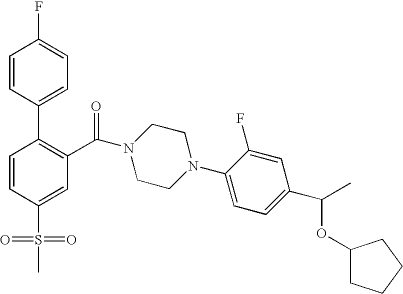 | 1-{4-[1-(cyclopentyloxy)ethyl]-2-fluorophenyl}-4-{[4′-fluoro-4-(methylsulfonyl)-2-biphenylyl]carbonyl}piperazine | 591(M + 23), |
| 87 | 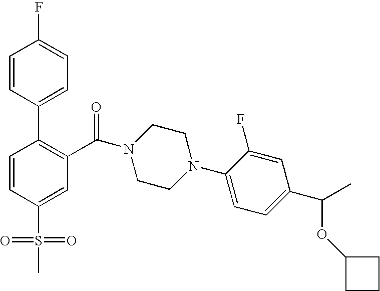 | 1-{4-[1-(cyclobutyloxy)ethyl]-2-fluorophenyl}-4-{[4′-fluoro-4-(methylsulfonyl)-2-biphenylyl]carbonyl}piperazine | 577(M + 23) |
| 88 | 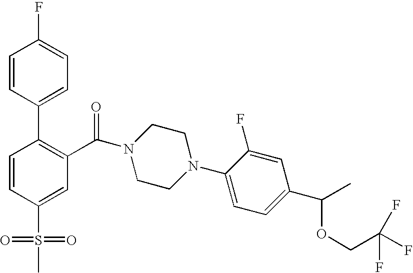 | 1-{[4′-fluoro-4-(methylsulfonyl)-2-biphenylyl]carbonyl}-4-(2-fluoro-4-{1-[(2,2,2-trifluoroethyl)oxy]ethyl}phenyl)piperazine | 605(M + 23) |
| 89 | 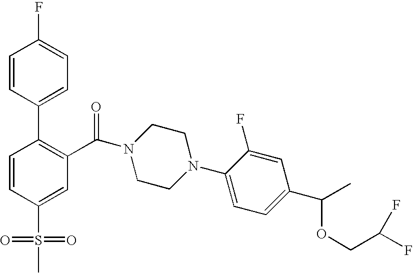 | 1-(4-{1-[(2,2-difluoroethyl)oxy]ethyl}-2-fluorophenyl)-4-{[4′-fluoro-4-(methylsulfonyl)-2-biphenylyl]carbonyl}piperazine | 587(M + 23) |
| 90 | 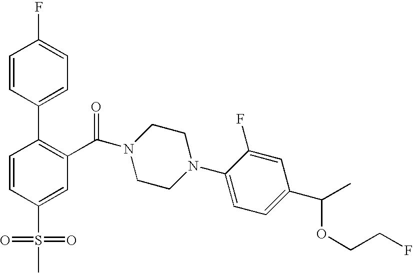 | 1-(2-fluoro-4-{1-[(2-fluoroethyl)oxy]ethyl}phenyl)-4-{[4′-fluoro-4-(methylsulfonyl)-2-biphenylyl]carbonyl}piperazine | 569(M + 23) |
| 91 | 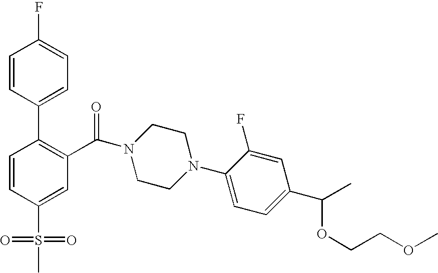 | 1-[2-fluoro-4-(1-{[2-(methyloxy)ethyl]oxy}ethyl)phenyl]-4-{[4′-fluoro-4-(methylsulfonyl)-2-biphenylyl]carbonyl}piperazine | 581(M + 23) |
| 92 | 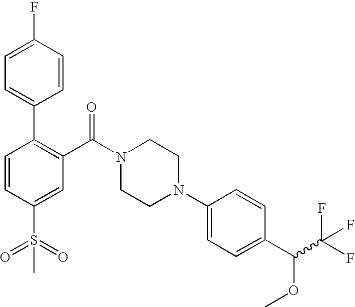 | 1-{[4′-fluoro-4-(methylsulfonyl)-2-biphenylyl]carbonyl}-4-{4-[2,2,2-trifluoro-1-(methyloxy)ethyl]phenyl}piperazine | 551(M + 1) |
| 93 | 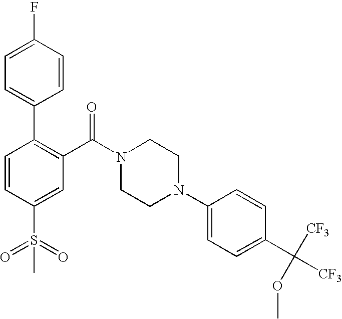 | 1-{[4′-fluoro-4-(methylsulfonyl)-2-biphenylyl]carbonyl}-4-{4-[2,2,2-trifluoro-1-(methyloxy)-1-(trifluoromethyl)ethyl]phenyl}piperazine | 619(M + 1) |
| 94 | 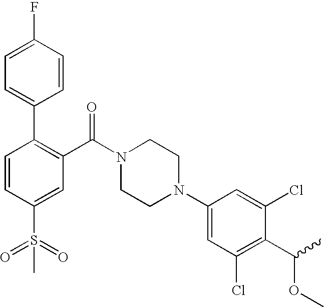 | 1-{3,5-dichloro-4-[1-(methyloxy)ethyl]phenyl}-4-{[4′-fluoro-4-(methylsulfonyl)-2-biphenylyl]carbonyl}piperazine | 587(M + 23) |
| 95 | 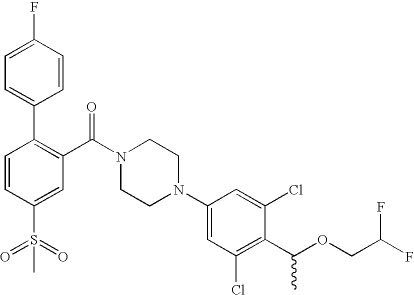 | 1-(3,5-dichloro-4-{1-[(2,2-difluoroethyl)oxy]ethyl}phenyl)-4-{[4′-fluoro-4-(methylsulfonyl)-2-biphenylyl]carbonyl}piperazine | 615(M + 1) |
| 96 | 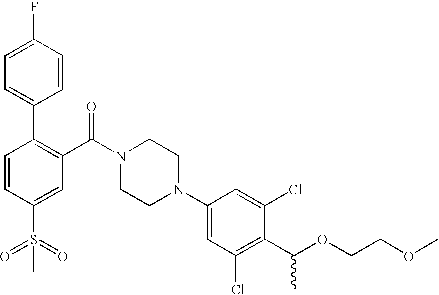 | 1-[3,5-dichloro-4-(1-{[2-(methyloxy)ethyl]oxy}ethyl)phenyl]-4-{[4′-fluoro-4-(methylsulfonyl)-2-biphenylyl]carbonyl}piperazine | 609(M + 1) |
| 97 | 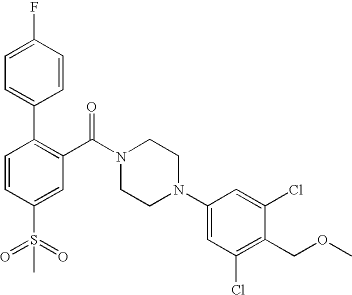 | 1-{3,5-dichloro-4-[(methyloxy)methyl]phenyl}-4-{[4′-fluoro-4-(methylsulfonyl)-2-biphenylyl]carbonyl}piperazine | 573(M + 23) |

### EXAMPLE 98

(2-{[2,6-Dichloro-4-(4-{[4-(methylsulfonyl)-2-biphenylyl]carbonyl}-1-piperazinyl)phenyl]oxy}ethyl)dimethylamine

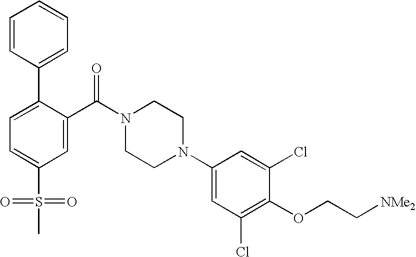

To a solution of 2,6-Dichloro-4-(4-{[4-(methylsulfonyl)-2-biphenylyl]carbonyl}-1-piperazinyl)phenol (0.15 g) in toluene (5 ml) and tetrahydrofuran (5 ml) was added sequentially triphenylphosphine (0.31 g), diisopropyl azodicarboxylate (0.24 ml) and (2-hydroxyethyl)dimethylamine (0.133 g). The reaction mixture was stirred at room temperature for 18 h, diluted with diethyl ether and washed with saturated ammonium chloride. The aqueous layer was extracted with diethyl ether and the combined organics dried (Na₂SO₄) and evaporated. Chromatography on silica gel eluting with 0-100% ethyl acetate in pentane gradient then 1-20% methanol in ethyl acetate afforded the title compound as a colourless gum. (0.13 g, 75%). LC/MS (ESI) Found 576 (M+1), 598 (M+23).

### EXAMPLE 99

### (2-{[2,6-Dichloro-4-(4-{[4-(methylsulfonyl)-2-biphenylyl]carbonyl}-1-piperazinyl)phenyl]oxy}butyl)dimethylamine

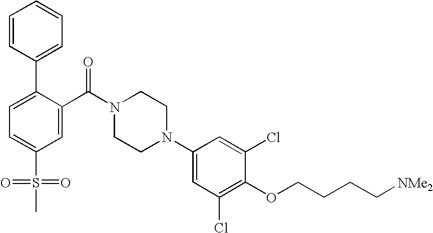

To a stirred solution of 1-{4-[(4-bromobutyl)oxy]-3,5-dichlorophenyl}-4-{[4-(methylsulfonyl)-2-biphenylyl]carbonyl}piperazine (0.14 g) and triethylamine (0.36 ml) in anhydrous dimethylformamide (3 ml) at room temperature under argon was added dimethylamine hydrochloride (0.179 g). After 71 h the mixture was diluted with diethyl ether (200 ml) and washed with saturated sodium bicarbonate (100 ml), water (100 ml) and brine (25 ml). The organic layer was dried (Na₂SO₄) and evaporated. The residue was applied to a 2 g SCX column and the column washed with DCM (20 ml) and methanol (20 ml). Elution with 1N ammonia in methanol and evaporation in vacuo afforded the title compound as a colourless gum (0.12 g, 90%).

LC/MS (ESI) Found 604 (M+1).

### EXAMPLE 100

### 1-{2-fluoro-4-[1-(methyloxy)ethyl]phenyl}-4-{[4′-fluoro-4-(ethylsulfonyl)-2-biphenylyl]carbonyl}piperazine

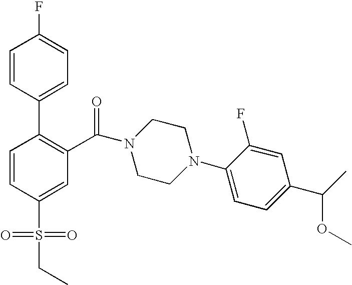

60% Sodium hydride dispersion in mineral oil (5 mg) was added to a stirred solution of (+)-1-[3-fluoro-4-(4-{[4′-fluoro-4-(methylsulfonyl)-2-biphenylyl]carbonyl}-1-piperazinyl)phenyl]ethanol (0.04 g) in anhydrous dimethylformamide (2 ml) at room temperature under argon. After 0.5 h methyl iodide (50 mg) was added and the mixture stirred for 4 h. Further 60% sodium hydride (5 mg), followed by methyl iodide (50 mg), 0.5 h later, were added and the mixture stirred for 18 h. Further 60% sodium hydride (5 mg), followed by methyl iodide (50 mg), 0.5 h later, were added and the mixture stirred for 4 h. Water (2 ml) was added and the reaction mixture evaporated in vacuo. The residue was partitioned between DCM and water (20 ml of each), the aqueous layer extracted with DCM (10 ml) and the combined organics dried (Na₂SO₄) and evaporated in vacuo. Chromatography on silica gel eluting with 0-100% ethyl acetate in pentane gradient afforded the title compound as a colourless foam. (0.018 g, 42%). LC/MS (ESI) Found 529 (M+1).

The following compounds were prepared by procedures similar to Example 18, Route B, using the appropriate alcohol.

| Example | | | LC/MS |
|---|---|---|---|
| No. | Structure | Name | (ESI) |
| 101 | 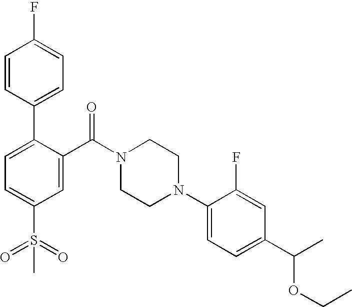 | 1-{4-[1-(ethyloxy)ethyl]-2-fluorophenyl}-4-{[4′-fluoro-4-(methylsulfonyl)-2-biphenylyl]carbonyl}piperazine | 551(M + 23) |
| 102 | 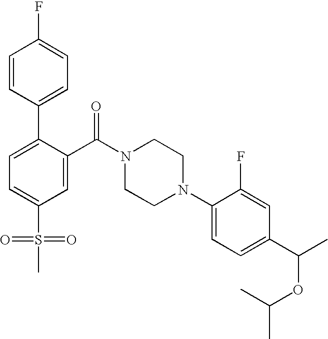 | 1-(2-fluoro-4-{1-[(1-methylethyl)oxy]ethyl}phenyl)-4-{[4′-fluoro-4-(methylsulfonyl)-2-biphenylyl]carbonyl}piperazine | 565(M + 23) |
| 103 | 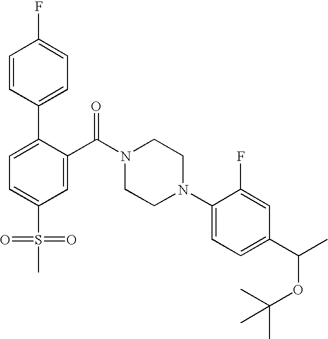 | 1-(4-{1-[(1,1-dimethylethyl)oxy]ethyl}-2-fluorophenyl)-4-{[4′-fluoro-4-(methylsulfonyl)-2-biphenylyl]carbonyl}piperazine | 579(M + 23) |
| 104 | 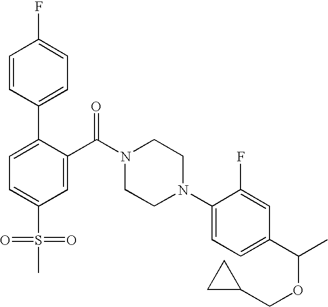 | 1-(4-{1-[cyclopropylmethyl)oxy]ethyl}-2-fluorophenyl)-4-{[4′-fluoro-4-(methylsulfonyl)-2-biphenylyl]carbonyl}piperazine | 577(M + 23) |

### EXAMPLE 105

### 1-(4-{[(Difluoromethyl)oxy]methyl}-2-fluorophenyl)-4-{[4′-fluoro-4-(methylsulfonyl)-2-biphenylyl]carbonyl}piperazine

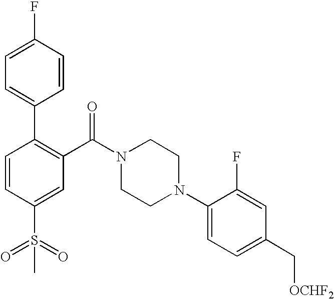

To [3-fluoro-4-(4-{[4′-fluoro-4-(methylsulfonyl)-2-biphenylyl]carbonyl}-1-piperazinyl)phenyl]methanol (100 mg) and sodium sulphate (2 mg) in acetonitrile (1 ml) at 45° C. under argon was added dropwise fluorosulfonyldifluoroacetic acid (18 mg) in acetonitrile (0.3 ml). After 4 h at 45° C. the resulting mixture was stirred at ambient temperature for 18 h before being reheated to 45° C. and further fluorosulfonyl difluoroacetic acid (42 mg) in acetonitrile (1 ml) added. After 2 h the reaction mixture was cooled and partitioned between dichloromethane and water. The organic phase was dried and evaporated in vacuo and the product thus obtained dissolved in 1:1 DMSO/MeCN (0.9 ml) and purified by mass directed autoprep hplc on a Waters C₁₈ 5 μM column 8(id 19×100 mm) eluting with 5-99% MeCN in water containing 0.1% formic acid. The product containing fractions were combined and evaporated; reevaporation from toluene (x3) afforded the title product as an amorphous solid (10 mg; 9%). LC/MS (ESI) Found 537 (M+1).

### EXAMPLE 106

### 1-[3-Fluoro-4-(4-{[4′-fluoro-4-(methylsulfonyl)-2-biphenylyl]carbonyl}-1-piperazinyl)phenyl]-1-propanone and 1-[3-fluoro-4-(4-{[4′-fluoro-4-(methylsulfonyl)-2-biphenylyl]carbonyl}-1-piperazinyl)phenyl]-1-propanol

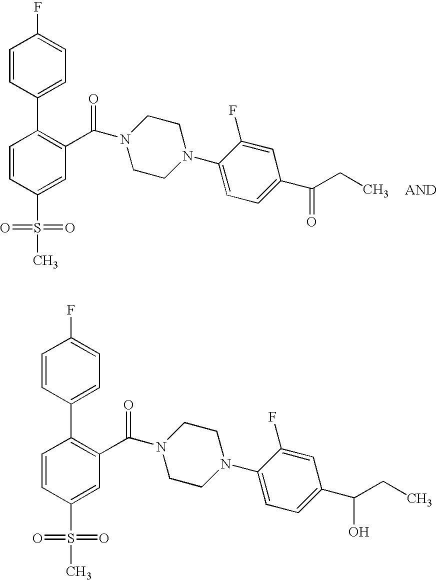

A solution of 3-fluoro-4-(4-{[4′-fluoro-4-(methylsulfonyl)-2-biphenylyl]carbonyl}-1-piperazinyl)benzaldehyde (500 mg, 1.03 mmol) in dry THF was treated dropwise with a solution of ethyl magnesium bromide (1.0 M in THF; 1.1 ml, 1.1 mmol) and the mixture stirred at room temp. under argon for 18 h. The mixture was evaporated in vacuo and partitioned between MDC and saturated aqueous NaHCO₃. The organics were evaporated and chromatographed (eluent 0-60% EtOAc/MDC) to afford 1-[3-Fluoro-4-(4-{[4′-fluoro-4-(methylsulfonyl)-2-biphenylyl]carbonyl}-1-piperazinyl)phenyl]-1-propanone, 93 mg, (18% yield).

LC/MS (ESI) Found 513 (M+1).

Further elution afforded the alcohol 1-[3-fluoro-4-(4-{[4′-fluoro-4-(methylsulfonyl)-2-biphenylyl]carbonyl}-1-piperazinyl)phenyl]-1-propanol, 205 mg, (39% yield).

LC/MS (ESI) Found 515 (M+1).

### EXAMPLE 107

### N-[4-(4-{[4′-fluoro-4-(methylsulfonyl)-2-biphenylyl]carbonyl}-1-piperazinyl)phenyl]-N-methylpropanamide

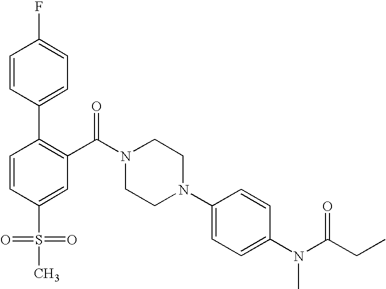

(i) A solution of 4-(4-{[4′-fluoro-4-(methylsulfonyl)-2-biphenylyl]carbonyl}-1-piperazinyl)aniline (400 mg, 0.88 mmol) in MDC (5 ml) was treated with triethylamine (0.14 ml, 1.0 mmol) followed by propionyl chloride (0.1 ml) and the mixture shaken in a Sarstedt tube at room temp. for 16 h. The mixture was washed with saturated aqueous NaHCO₃, separated through PhaseSep cartridge and evaporated in vacuo to give a white solid 360 mg.

(ii) 61 mg (0.12 mmol) of the solid was dissolved in DMF and treated with sodium hydride (5 mg, 0.13 mmol) followed by methyl iodide (18 mg, 0.13 mmol) and the mixture shaken at room temp. for 16 h. The mixture was partitioned (EtOAc/H₂O), the organics further washed with H₂O (x2), dried (Na₂SO₄) and evaporated. Chromatography (eluent 0-100% EtOAc/pentane) afforded the title compound as a white solid 22 mg, 35%.

δ_{H} (400 MHz, CDCl₃) 8.05 (2H, m), 7.64 (1H, d), 7.51 (2H, m), 7.19 (2H, t), 7.03 (2H, d), 6.76 (2H, d), 3.79 (1H, m), 3.71 (1H, m), 3.20 (3H, s), 3.14 (2H, m), 3.13 (3H, s), 2.89 (3H, m), 2.29 (1H, m), 2.05 (2H, q), 1.02 (3H, t).

LC/MS (ESI) Found 524 (M+1).

The following compounds were prepared by procedures similar to Example 107,

| | | | |
|---|---|---|---|
| 108 | 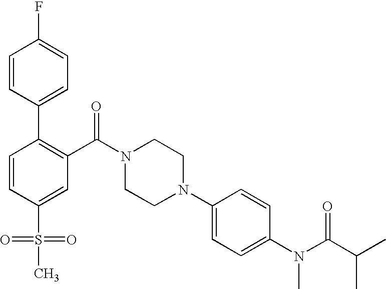 | N-[4-(4-{[4′-fluoro-4-(methylsulfonyl)-2-biphenylyl]carbonyl}-1-piperazinyl)phenyl]-N,2-dimethylpropanamide | 538(M + 1) |
| 109 | 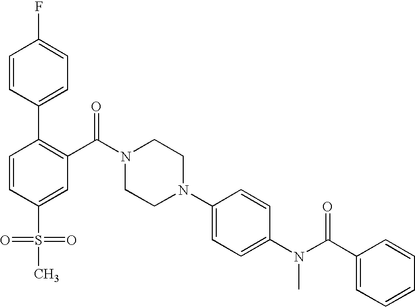 | N-[4-(4-{[4′-fluoro-4-(methylsulfonyl)-2-biphenylyl]carbonyl}-1-piperazinyl)phenyl]-N-methylbenzamide | 572(M + 1) |

### EXAMPLE 110

### N-[3-Fluoro-4-(4-{[4′-fluoro-4-(methylsulfonyl)-2-biphenylyl]carbonyl}-1-piperazinyl)phenyl]acetamide

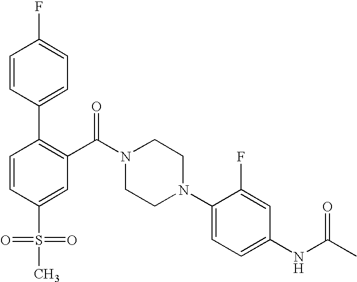

A solution of 3-fluoro-4-(4-{[4′-fluoro-4-(methylsulfonyl)-2-biphenylyl]carbonyl}-1-piperazinyl)aniline (200 mg, 0.42 mmol) MDC (10 ml) was treated with acetic anhydride (0.05 ml, 0.5 mmol) and stirred at room temp for 16 h. The mixture was washed with saturated aqueous NaHCO₃ and the organics dried through PhaseSep cartridge to afford on evaporation the title compound as a white solid 173 mg, 80%.

LC/MS (ESI) Found 514 (M+1)

### EXAMPLE 111

### N-[3-fluoro-4-(4-{[4′-fluoro-4-(methylsulfonyl)-2-biphenylyl]carbonyl}-1-piperazinyl)phenyl]-N-methylacetamide

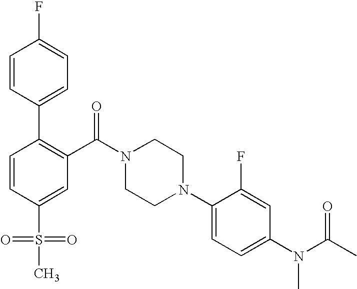

The compound was prepared by procedures similar to Example 107, part (ii), from N-[3-fluoro-4-(4-{[4′-fluoro-4-(methylsulfonyl)-2-biphenylyl]carbonyl}-1-piperazinyl)phenyl]acetamide (125 mg, 0.24 mmol) to afford the title compound as a white solid 70 mg, (55% yield).

δ_{H} (400 MHz, CDCl₃) 8.06 (2H, m), 7.64 (1H, d), 7.52 (2H, m), 7.21 (2H, t), 6.88 (2H, m), 6.75 (1H, t), 3.82 (1H, m), 3.73 (1H, m), 3.20 (3H, s), 3.14 (1H, m), 3.13 (3H, s), 3.05 (1H, m), 2.84 (3H, m), 2.18 (1H, m), 1.86 (3H, s).

LC/MS (ESI) Found 528 (M+1).

### EXAMPLE 112

### 1-[3,5-Dichloro-4-(methyloxy)phenyl]-4-{[5-(methylsulfonyl)-2-(3-thienyl)phenyl]carbonyl}piperazine

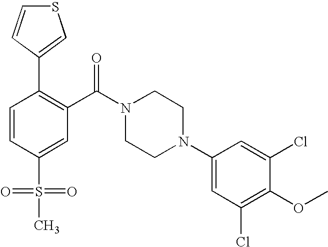

5-(Methylsulfonyl)-2-(3-thienyl)benzoic acid (0.200 g) and 1-[3,5-dichloro-4-(methyloxy)phenyl]piperazine hydrochloride (0.210 g) were dissolved in DMF (10 ml) and added excess of DIPEA (0.8 ml) and HATU (0.270 g). The reaction mixture was stirred at room temperature for 16 hours under an argon atmosphere. The reaction mixture was then partitioned between ethyl acetate and water. The organic solution was dried (Na₂SO₄) and evaporated to yield the crude product, which was purified by silica gel column chromatography, eluting with a gradient of 0 to 100% ethyl acetate in N-pentane.

The title compound was obtained as a white solid (91% yield), LC/MS (ESI) Found 525, 527. 2 peaks (M+1).

### EXAMPLE 113

### 1-[3,5-Dichloro-4-(methyloxy)phenyl]-4-{[5-(methylsulfonyl)-2-(2-thienyl)phenyl]carbonyl}piperazine

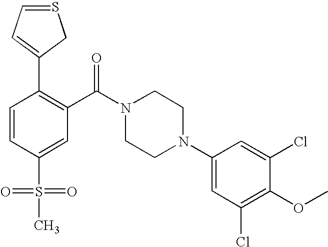

The title compound was prepared from 5-(methylsulfonyl)-2-(2-thienyl)benzoic acid and 1-[3,5-dichloro-4-(methyloxy)phenyl]piperazine hydrochloride according to the procedure described in Example 112. (41% yield).

LC/MS (ESI) Found 527 (M+1).

### EXAMPLE 114

### 1-[2-Fluoro-4-(methylsulfonyl)phenyl]-4-{[4-(methylsulfonyl)-2-biphenylyl]carbonyl}piperazine

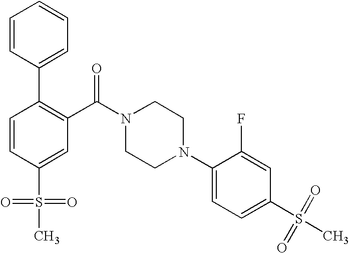

The title compound was prepared from 4-(methylsulfonyl)-2-biphenylcarboxylic acid and 1-[2-fluoro-4-(methylsulfonyl)phenyl]piperazine according to the procedure described in Example 112. (83% yield). LC/MS (ESI) Found 517.1 (M+1).

### EXAMPLE 115

### 1-[3,5-Difluoro-4-(methyloxy)phenyl]-4-{[4-(methylsulfonyl)-2-biphenylyl]carbonyl}piperazine

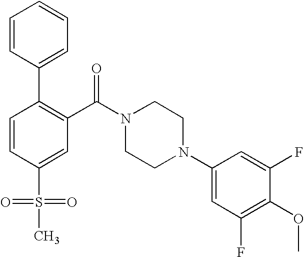

The title compound was prepared from 4-(methylsulfonyl)-2-biphenylcarboxylic acid and 1-[3,5-difluoro-4-(methyloxy)phenyl]piperazine according to the procedure described in Example 112. (66% yield). LC/MS (ESI) Found 487.24 (M+1).

### EXAMPLE 116

### 1-[3,5-Difluoro-4-(methyloxy)phenyl]-4-{[4′-fluoro-4-(methylsulfonyl)-2-biphenylyl]carbonyl}piperazine

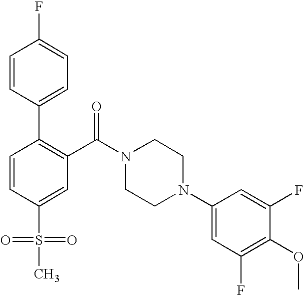

The title compound was prepared from 4′-fluoro-4-(methylsulfonyl)-2-biphenylcarboxylic acid and 1-[3,5-difluoro-4-(methyloxy)phenyl]piperazine according to the procedure described in Example 112. (34% yield). LC/MS (ESI) Found 505.2 (M+1).

### EXAMPLE 117

### 1-[3-Chloro-5-fluoro-4-(methyloxy)phenyl]-4-{[4′-fluoro-4-(methylsulfonyl)-2-biphenylyl]carbonyl}piperazine

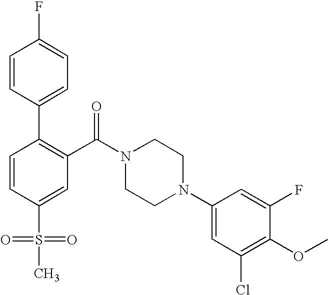

The title compound was prepared from 4′-fluoro-4-(methylsulfonyl)-2-biphenylcarboxylic acid and 1-[3-chloro-5-fluoro-4-(methyloxy)phenyl]piperazine according to the procedure described in Example 112. (44% yield). LC/MS (ESI) Found 521.2 (M+1).

### EXAMPLE 118

### 1-[6-(4-{[4′-Fluoro-4-(methylsulfonyl)-2-biphenylyl]carbonyl}-1-piperazinyl)-3-pyridinyl]ethanone

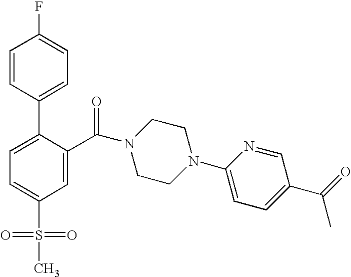

The title compound was prepared from 4′-fluoro-4-(methylsulfonyl)-2-biphenylcarboxylic acid and 1-[6-(1-piperazinyl)-3-pyridinyl]ethanone according to the procedure described in Example 112. (85% yield). LC/MS (ESI) Found 482.2 (M+1).

### EXAMPLE 119

### 1-[6-(4-{[4′-Fluoro-4-(methylsulfonyl)-2-biphenylyl]carbonyl}-1-piperazinyl)-3-pyridinyl]ethanol

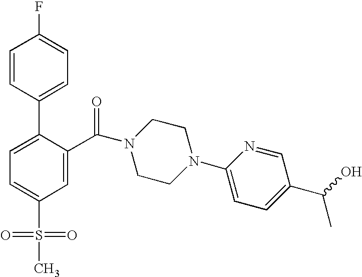

To a mixture of 1-[6-(4-{[4′-fluoro-4-(methylsulfonyl)-2-biphenylyl]carbonyl}-1-piperazinyl)-3-pyridinyl]ethanone (0.750 g), methanol (100 ml) was added excess of sodium borohydride portionwise over several minutes (bubbling, exothermic) (0.260 g), and the mixture was heated for 6 hours at 60° C. After cooling, a small amount of water was added, then evaporated off methanol under reduced pressure. The residue was then partitioned between dichloromethane and water, The organic layer was dried (Na₂SO₄) and evaporated, the residue was chromatographed over silica gel. Elution with a gradient of 0 to 10% methanol in dichlorometnane gave the title compound as a white solid (65% yield). LC/MS (ESI) Found 466.3 (M+1−H₂O).

### EXAMPLE 120

### 1-{[4′-Fluoro-4-(methylsulfonyl)-2-biphenylyl]carbonyl}-4-{5-[1-(methyloxy)ethyl]-2-pyridinyl}piperazine

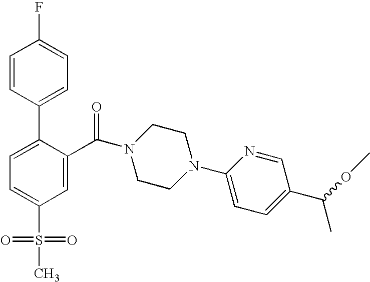

A mixture of 1-[6-(4-{[4′-fluoro-4-(methylsulfonyl)-2-biphenylyl]carbonyl}-1-piperazinyl)-3-pyridinyl]ethanol (0.130 g), PTSA (0.024 g), methanol (0.8 ml) in toluene 10 ml) and dioxane (10 ml) was heated at 80° C. for 2 hours. After cooling, evaporated off solvent under reduced pressure. The residue was then partitioned between sodium bicarbonate solution and dichloromethane. The organic solution was washed with water, dried (Na₂SO₄) and evaporated to yield the crude product, which was purified by silica gel column chromatography, eluting with a gradient of 10 to 100% ethyl acetate in N-pentane. The title compound was obtained as a white solid (64% yield), LC/MS (ESI) Found 520.2 (M+Na).

### EXAMPLE 121

### 1-[3-Fluoro-4-(4-{[4-(methylsulfonyl)-2-biphenylyl]carbonyl}-1-piperazinyl)phenyl]ethanone

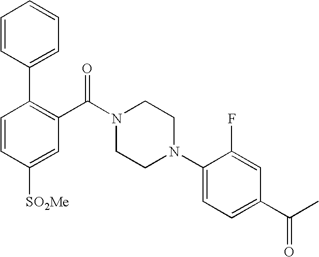

EDC (694 mg, 3.62 mmol) was added in one portion to a stirring solution of the acid 4-(methylsulfonyl)-2-biphenylcarboxylic acid (514 mg, 1.86 mmol), the piperazine 1-[3-fluoro-4-(1-piperazinyl)phenyl]ethanone (410 mg, 1.84 mmol) and Et₃N (300 μL, 2.24 mmol) in DCM (50 mL). Stirring was continued at room temperature for 16 h and the water (100 mL) added. The organic layer was removed and the aqueous extracted with EtOAc (2×40 mL). The combined organic layers were dried (MgSO₄) and the solvent removed in vacuo. Purification by column chromatography (Flashmaster II, 50 g Isolute flash column, gradient elution 100% pentane to 67% EtOAc over 7 min, 67% EtOAc for 10 min, 67% EtOAc to 100% EtOAc over 10 min) gave 1-[3-fluoro-4-(4-{[4-(methylsulfonyl)-2-biphenylyl]carbonyl}-1-piperazinyl)phenyl]ethanone (594 mg, 68%) as a white solid. δ_{H} (400 MHz) 8.07-8.05 (2H, m), 7.68-7.44 (8H, m), 6.70 (1H, t, J 8.4), 3.84-3.81 (1H, m), 3.71-3.68 (1H, m), 3.19-3.12 (2H, m (obscured)), 3.14 (3H, s, SO₂CH₃), 2.84-2.77 (3H, m), 2.52 (3H, s, COCH₃), 1.26 (1H, t, J 8.4); LC/MS (Ammonium bicarbonate ES+) Found 481 (M+H, T_{R} 2.59 min)

### EXAMPLE 122

### (1E)-1-[3-fluoro-4-(4-{[4-(methylsulfonyl)-2-biphenylyl]carbonyl}-1-piperazinyl)phenyl]ethanone O-methyloxime

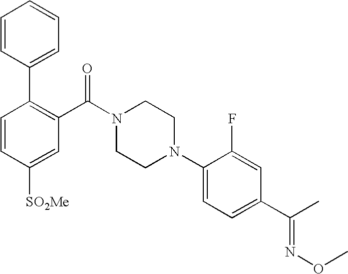

Methoxyamine hydrochloride (23 mg, 0.27 mmol) was added in one portion to a stirring solution of the 1-[3-fluoro-4-(4-{[4-(methylsulfonyl)-2-biphenylyl]carbonyl}-1-piperazinyl)phenyl]ethanone (106 mg, 0.22 mmol) in pyridine in MeOH (5 mL, 15% v/v). Stirring was continued at room temperature for 20 h and the solvent removed in vacuo. The product was dissolved in water (20 mL) and extracted with Et₂O (2×25 mL). The combined organic layers were reduced and recrystallisation from Et₂O gave (1E)-1-[3-fluoro-4-(4-{[4-(methylsulfonyl)-2-biphenylyl]carbonyl}-1-piperazinyl)phenyl]ethanone O-methyloxime (52 mg, 49%) as a white solid. δ_{H} (400 MHz) 8.06-8.04 (2H, m), 7.67-7.65 (1H, m), 7.55-7.43 (5H, m), 7.36-7.26 (2H, m (obscured)), 6.65 (1H, t, J 8.4), 3.97 (3H, s, OCH₃), 3.87-3.78 (1H, m), 1.71-1.63 (1H, m), 3.18-3.11 (1H, m (obscured)), 3.13 (3H, s, SO₂CH₃), 3.10-3.01 (1H, m), 2.87-2.79 (1H, m), 2.76-2.66 (2H, m), 2.15 (3H, s, C(N)CH₃), 1.96-1.88 (1H, m); LC/MS (Ammonium bicarbonate ES+) Found 510 (M+H, T_{R} 2.90 min)

### EXAMPLE 123

### 1-[3-fluoro-4-(4-{[4-(methylsulfonyl)-2-biphenylyl]carbonyl}-1-piperazinyl)phenyl]ethanol

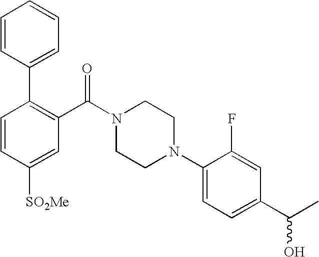

Sodium borohydride (8.86 mg, 0.23 mmol) was added in one portion to a stirring solution of 1-[3-fluoro-4-(4-{[4-(methylsulfonyl)-2-biphenylyl]carbonyl}-1-piperazinyl)phenyl]ethanone (102 mg, 0.21 mmol) in MeOH (5 mL). Stirring was continued at room temperature for 18 h and the solvent removed in vacuo. The product was partitioned between water (25 mL) and EtOAc (25 mL) and the aqueous further extracted with EtOAc (25 mL). The combined organic layers were dried (MgSO₄) and the solvent removed in vacuo. Purification by column chromatography (Flashmaster II, 5 g Isolute flash column, gradient elution 100% pentane to 67% EtOAc over 4 min, 67% EtOAc for 5 min, 67% EtOAc to 100% EtOAc over 4 min, 100% EtOAc for 4 min) gave 1-[3-fluoro-4-(4-{[4-(methylsulfonyl)-2-biphenylyl]carbonyl}-1-piperazinyl)phenyl]ethanol (96 mg, 96%) as a white solid. δ_{H} (400 MHz) 8.06-8.04 (2H, m), 7.67-7.65 (1H, m), 7.54-7.43 (5H, m), 7.05-7.00 (2H, m), 6.65 (1H, t, J 8.4), 4.82 (1H, q, J 6.4, C(OH)H), 3.80-3.76 (1H, m), 3.72-3.69 (1H, m), 3.16-3.09 (1H, m (obscured)), 3.13 (3H, s, SO₂CH₃), 3.03-2.96 (1H, m), 2.84-2.79 (1H, m), 2.68-2.63 (2H, m), 1.94-1.90 (1H, m), 1.80 (1H, s); LC/MS (Ammonium bicarbonate ES+) Found 505 (M+Na, T_{R} 2.42 min)

### EXAMPLE 124

### 1-{2-fluoro-4-[1-(methyloxy)ethyl]phenyl}-4-{[4-(methylsulfonyl)-2-biphenylyl]carbonyl}piperazine

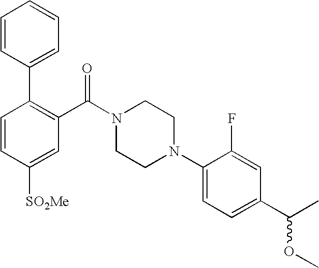

Sodium hydride (9.3 mg, 0.23 mmol) was added in one portion to a stirring solution of 1-[3-fluoro-4-(4-{[4-(methylsulfonyl)-2-biphenylyl]carbonyl}-1-piperazinyl)phenyl]ethanol (91 mg, 0.19 mmol) in DMF (5 mL). Stirring at room temperature was continued for 10 min and MeI (16 μL, 0.26 mmol) was added dropwise over 30 s. Stirring was continued at room temperature for 3 h and the reaction mixture was quenched with NH₃ in MeOH (2 M). The solvent was removed in vacuo and purification carried out by column chromatography (Flashmaster II, 5 g isolute flash column, gradient elution 100% pentane to 45% EtOAc over 3 min, 45% EtOAc to 60% EtOAc over 15 min, 60% EtOAc to 100% EtOAc over 4 min). Further purification by mass directed HPLC to give methyl ether 1-{2-fluoro-4-[1-(methyloxy)ethyl]phenyl}-4-{[4-(methylsulfonyl)-2-biphenylyl]carbonyl}piperazine (23 mg, 23%) as a white solid. δ_{H} (400 MHz) 8.05-8.03 (2H, m), 7.68-7.65 (1H, m), 7.55-7.42 (5H, m), 6.98-6.94 (2H, m), 6.65 (1H, t, J 8.4), 4.20 (1H, q, J 6.4, C(OCH₃)H), 3.82-3.68 (2H, m), 3.19 (3H, s, OCH₃), 3.16-3.08 (1H, m (obscured)), 3.13 (3H, s, SO₂CH₃), 3.05-2.97 (1H, m), 2.84-2.78 (1H, m), 2.71-2.63 (2H, m), 1.98-1.92 (1H, m), 1.38 (3H, d, J 6.4, C(OCH₃)CH₃); LC/MS (Ammonium bicarbonate ES+) Found 519 (M+Na, T_{R} 2.74 min)

### EXAMPLE 125

### 1-{2-Fluoro-4-[1-(ethyloxy)ethyl]phenyl}-4-{[4-(methylsulfonyl)-2-biphenylyl]carbonyl}piperazine

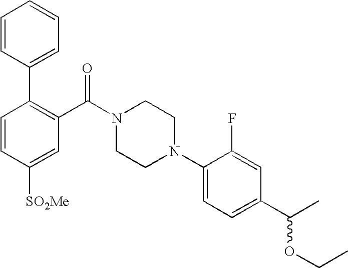

The title compound was synthesised using procedure of Example 124.

LC/MS (Ammonium bicarbonate ES+) Found 533 (M+Na, T_{R} 2.96 min).

### EXAMPLE 126

### 1-{[4′-Fluoro-4-(methylsulfonyl)-2-biphenylyl]carbonyl}-4-[4-(1H-pyrazol-1-yl)phenyl]piperazine

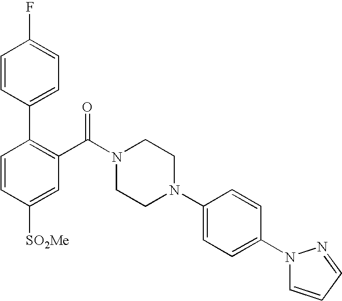

HATU (92 mg, 0.24 mmol) was added in one portion to a stirring solution of the acid 4′-fluoro-4-(methylsulfonyl)-2-biphenylcarboxylic acid (55 mg, 0.19 mmol) and Hünig's Base (90 μL, 0.53 mmol) in DMF (1 mL). After stirring for 8 min the 1-[4-(1H-pyrazol-1-yl)phenyl]piperazine (40 mg, 0.18 mmol) was added and stirring continued for 16 h. The volatile components were removed in vacuo and purification was carried out by column chromatography (Biotage Horizon, Biotage 12+M column, gradient elution 100% EtOAc for 48 mL, 100% EtOAc to 5% MeOH over 48 mL, 5% MeOH for 72 mL, 5% MeOH to 10% MeOH over 72 mL). Further purification by mass directed HPLC gave 1-{[4′-fluoro-4-(methylsulfonyl)-2-biphenylyl]carbonyl}-4-[4-(1H-pyrazol-1-yl)phenyl]piperazine (60 mg, 68%) as a white solid. δ_{H} (400 MHz) 8.06-8.03 (2H, m), 7.82-7.81 (1H, m), 7.68-7.63 (2H, m), 7.56-7.50 (4H, m), 7.18 (2H, t, J 8.4), 6.84 (2H, d, J 8.8), 6.43-6.42 (1H, m), 3.84-3.70 (2H, m), 3.18-3.13 (2H, m (obscured)), 3.13 (3H, s, SO₂CH₃), 2.92-2.79 (3H, m), 2.30-2.21 (1H, m); LC/MS (Ammonium bicarbonate ES+) Found 505 (M+H, T_{R} 2.64 min).

The following compounds were synthesised using the procedure described in Example 126.

| Example | | | LC/MS |
|---|---|---|---|
| No. | Structure | Name | (ESI) |
| 127 | 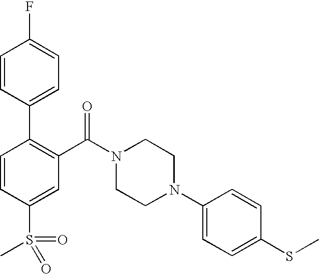 | 1-{[4′-fluoro-4-(methylsulfonyl)-2-biphenylyl]carbonyl}-4-[4-(methylthio)phenyl]piperazine | Found485(M + 1) |
| 128 | 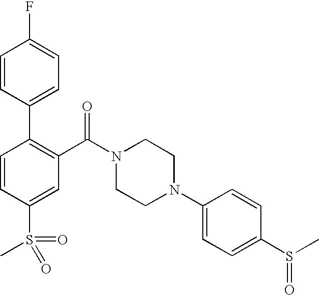 | 1-{[4′-fluoro-4-(methylsulfonyl)-2-biphenylyl]carbonyl}-4-[4-(methylsulfinyl)phenyl]piperazine | Found501(M + 1) |
| 129 | 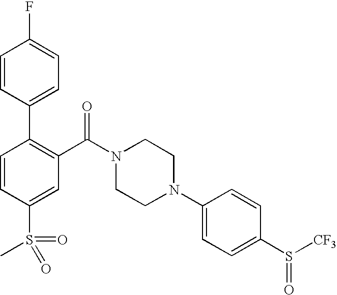 | 1-{[4′-fluoro-4-(methylsulfonyl)-2-biphenylyl]carbonyl}-4-{4-[(trifluoromethyl)sulfinyl]phenyl}piperazine | Found555(M + 1) |

### EXAMPLE 130

### 1-{3,5-Difluoro-4-[(methyloxy)methyl]phenyl}-4-{[2′-fluoro-4-(methylsulfonyl)-2-biphenylyl]carbonyl}piperazine

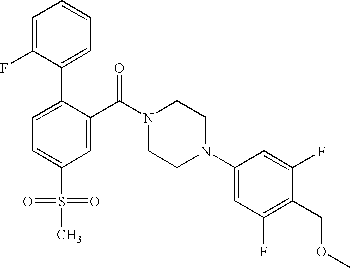

The title compound was prepared from [2,6-difluoro-4-(4-{[2′-fluoro-4-(methylsulfonyl)-2-biphenylyl]carbonyl}-1-piperazinyl)phenyl]methanol by the procedure described in Example 120, (43% yield).

LC/MS (ESI) Found 487.3 (M+1−MeOH).

### EXAMPLE 131

### 1-{3,5-Difluoro-4-[(methyloxy)methyl]phenyl}-4-{[4′-fluoro-4-(methylsulfonyl)-2-biphenylyl]carbonyl}piperazine

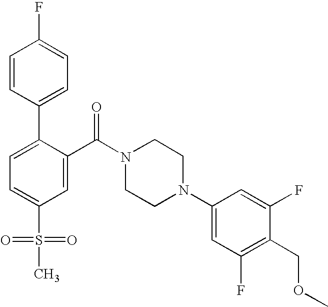

The title compound was prepared from [2,6-difluoro-4-(4-{[4′-fluoro-4-(methylsulfonyl)-2-biphenylyl]carbonyl}-1-piperazinyl)phenyl]methanol by the procedure described in Example 120, (32% yield).

LC/MS (ESI) Found 487.3 (M+1−MeOH), 541.3 (M+Na).

1-17. (canceled)

18. A compound of formula (I) or a salt thereof: 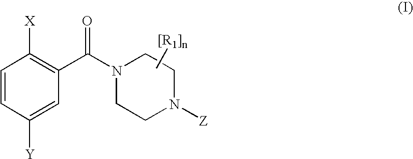
wherein
X is selected from the group consisting of C₅₋₁₁aryl and C₄₋₁₀heteroaryl, said C₅₋₁₁aryl and C₄₋₁₀heteroaryl being unsubstituted or substituted with one or more groups selected from the group consisting of halogen, hydroxy, cyano, C₁₋₄alkyl, C₁₋₄alkoxy, haloC₁₋₄alkyl, haloC₁₋₄alkoxy and C₁₋₄alkylthio.
Y is —S(O)ₘR₂ or —SO₂NR₃R₄ wherein
m is 1 or 2; and
R₂ is selected from the group consisting of C₁₋₆alkyl, C₃₋₇cycloalkyl, C₅₋₁₁aryl and C₄₋₁₀heteroaryl, where the C₁₋₆alkyl, C₃₋₇cycloalkyl, C₅₋₁₁aryl or C₄₋₁₀heteroaryl groups are unsubstituted or substituted with one or two groups selected from the group consisting of halo, C₁₋₄alkoxy and C₁₋₄haloalkoxy;
R₃ and R₄ are independently selected from the group consisting of hydrogen and C₁₋₆alkyl, where the C₁₋₆alkyl is unsubstituted or substituted with one or more groups selected from the group consisting of halo, C₁₋₄alkoxy and C₁₋₄haloalkoxy;
n is 0, 1 or 2,
each R₁ is independently selected from the group consisting of C₁₋₆alkyl and C₁₋₆haloalkyl; and
Z is:
Z′: 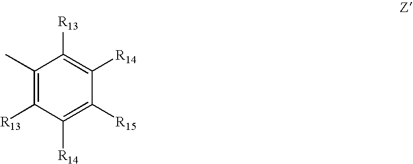
wherein each R₁₃ is independently selected from the group consisting of hydrogen, halogen, hydroxy, cyano, amino, C₂₋₆alkyl, C₁₋₄alkoxy, haloC₁₋₄alkyl, haloC₁₋₄alkoxy, C₆₋₁₁₁arylC₁₋₄alkoxy, C₁₋₄alkylthio, C₁₋₄alkoxyC₁₋₄alkyl, C₁₋₄haloalkoxyC₁₋₄alkyl, halohydroxyC₁₋₄alkyl, C₁₋₄alkoxyC₁₋₄alkoxyC₁₋₄alkyl, C₁₋₄alkoxyhaloC₁₋₄alkyl, C₃₋₆cycloalkylC₁₋₄alkyl, C₃₋₆cycloalkylC₁₋₄alkyl substituted by one or more C₁₋₄alkoxy groups in the C₁₋₄alkyl portion, C₃₋₆cycloalkyl, C₃₋₆cycloalkylC₁₋₄alkoxy, C₃₋₆cycloalkylhydroxyC₁₋₄alkyl, C₃₋₆cycloalkylC₁₋₄acyl, C₃₋₆cycloalkylC₁₋₄alkoxyC₁₋₄alkyl, C₃₋₆cycloalkoxyC₁₋₄alkyl, C₁₋₄alkanoyl, C₁₋₄haloalkanoyl, C₁₋₄alkoxycarbonyl, C₁₋₄alkoxycarbonylC₁₋₄alkyl, C₁₋₄alkylsulfonyl, C₁₋₄haloalkylsulfonyl, C₁₋₄alkylsulfinyl, C₁₋₄haloalkylsulfinyl, C₁₋₄alkylsulfonyloxy, C₁₋₄alkylsulfonylC₁₋₄alkyl, C₆₋₁₁arylsulfonyl, C₆₋₁₁arylsulfonyloxy, C₆₋₁₁arylsulfonylC₁₋₄alkyl, C₁₋₄alkylsulfonamido, C₄₋₉heteroarylsulfonyl, C₁₋₄alkylsulfonamidoC₁₋₄alkyl, C₁₋₄alkylamidoC₁₋₄alkyl, C₆₋₁₁arylsulfonamido, C₆₋₁₁arylcarboxamido, C₆₋₁₁arylsulfonamidoC₁₋₄alkyl, C₆₋₁₁arylcarboxamidoC₁₋₄alkyl, C₆₋₁₁aroyl, C₆₋₁₁aroylC₁₋₄alkyl, C₆₋₁₁arylC₁₋₄alkanoyl, formyl, C₁₋₄acyl, haloC₁₋₄acyl, arylC₁₋₄alkoxyC₁₋₄alkyl, C₆₋₁₁aryl, C₆₋₁₁aryl substituted by one to three groups selected from the group consisting of C₁₋₄alkyl, hydroxy, halogen, C₁₋₄alkoxy, C₁₋₄acyl and trifluoromethyl; C₆₋₁₁arylC₁₋₄alkyl, C₄₋₁₀heteroaryl, C₄₋₁₀heteroaryl substituted by one to three C₁₋₄alkyl groups, C₁₋₄alkylaminoC₁₋₄alkyl, a group —NR_{9′}R_{10′}, —(CH₂)ₚCONR₉R₁₀, —(CH₂)ₚSO₂NR₉R₁₀ or —(CH₂)ₚNR₉SO₂R₁₀, —CR_{9′}═NR_{10′}, —CR_{9′}═NOR_{10′}, —CR_{9′}═C(CN)₂—CR_{9′}═CH(CN), (CH₂)_{q}NR_{9′}R_{10′} and —O(CH₂)_{q}NR_{9′}R_{10′} wherein
each R₉ and R₁₀ is independently C₁₋₄alkyl, or R₉R₁₀ forms part of a C₃₋₆azacyloalkane or C₃₋₆(2-, 3- or 4-oxo)azacycloalkane ring
each R_{9′} and R_{10′} is independently selected from the group consisting of R₉ and R₁₀ and hydrogen;
each R_{9″} and R_{10″} is independently selected from the group consisting of the group consisting of R_{9′} and R_{10′} and C₁₋₄alkanoyl;
p is 0, 1, 2, 3 or 4;
q is 2, 3 or 4;
each R₁₄ is independently selected from the group consisting of hydrogen, halogen, hydroxy, cyano, amino, nitro, C₁₋₆alkyl, C₁₋₄alkoxy, haloC₁₋₄alkyl, haloC₁₋₄alkoxy, C₆₋₁₁arylC₁₋₄alkoxy, C₁₋₄alkylthio, hydroxyC₁₋₄alkyl, C₁₋₄haloalkoxyC₁₋₄alkyl, halohydroxyC₁₋₄alkyl, C₁₋₄alkoxyC₁₋₄alkoxyC₁₋₄alkyl, C₁₋₄alkoxyhaloC₁₋₄alkyl, C₃₋₆cycloalkylC₁₋₄alkyl, C₃₋₆cycloalkylC₁₋₄alkyl substituted by one or more C₁₋₄alkoxy groups in the C₁₋₄alkyl portion, C₃₋₆cycloalkyl, C₃₋₆cycloalkylC₁₋₄alkoxy, C₃₋₆cycloalkylhydroxyC₁₋₄alkyl, C₃₋₆cycloalkylC₁₋₄acyl, C₃₋₆cycloalkylC₁₋₄alkoxyC₁₋₄alkyl, C₃₋₆cycloalkoxyC₁₋₄alkyl, C₁₋₄alkanoyl, C₁₋₄haloalkanoyl, C₁₋₄alkoxycarbonyl, C₁₋₄alkoxycarbonylC₁₋₄alkyl, C₁₋₄alkylsulfonyl, C₁₋₄haloalkylsulfonyl, C₁₋₄alkylsulfinyl, C₁₋₄haloalkylsulfinyl, C₁₋₄alkylsulfonyloxy, C₁₋₄alkylsulfonylC₁₋₄alkyl, C₆₋₁₁arylsulfonyl, C₆₋₁₁arylsulfonyloxy, C₆₋₁₁arylsulfonylC₁₋₄alkyl, C₁₋₄alkylsulfonamido, C₄₋₉heteroarylsulfonyl, C₁₋₄alkylsulfonamidoC₁₋₄alkyl, C₆₋₁₁arylsulfonamidoC₁₋₄alkyl, C₆₋₁₁aroyl, C₆₋₁₁aroylC₁₋₄alkyl, C₆₋₁₁arylC₁₋₄alkanoyl, formyl, C₁₋₄acyl, haloC₁₋₄acyl, arylC₁₋₄alkoxyC₁₋₄alkyl, C₆₋₁₁aryl, C₆₋₁₁aryl substituted by one to three groups selected from the group consisting of C₁₋₄alkyl, hydroxy, halogen, C₁₋₄alkoxy, C₁₋₄acyl and trifluoromethyl; C₆₋₁₁arylC₁₋₄alkyl, C₄₋₁₀heteroaryl, C₄₋₁₀heteroaryl substituted by one to three groups selected from the group consisting of C₁₋₄alkyl groups, C₁₋₄alkylaminoC₁₋₄alkyl, a group —NR_{9″}R_{10″}, —(CH₂)ₚSO₂NR_{9′}R_{10′} or —(CH₂)ₚNR_{10′}SO₂R_{9′}, —CR_{9′}═NR_{10′}, —CR_{9′}═NOR_{10′}, —CR_{9′}═C(CN)₂, —CR_{9′}═CH(CN), (CH₂)_{q}NR_{9′}R_{10′}, and —O(CH₂)_{q}NR_{9′}R_{10′}, wherein
each R₉ and R₁₀ is independently C₁₋₄alkyl, or R₉R₁₀ forms part of a C₃₋₆azacyloalkane or C₃₋₆(2-, 3- or 4-oxo)azacycloalkane ring;
each R_{9′} and R_{10′} is independently selected from the group consisting of R₉ and R₁₀ and hydrogen;
each R_{9″} and R_{10″} is independently selected from the group consisting of R_{9′} and R_{10′} and C₁₋₄alkanoyl;
p is 0, 1, 2, 3 or 4;
q is 2, 3 or 4;
R₁₅ is selected from the group consisting of hydrogen, halogen, hydroxy, cyano, nitro, C₁₋₆alkyl, C₂₋₄alkenyl, C₁₋₄alkoxy, haloC₁₋₄alkyl, haloC₁₋₄alkoxy, C₆₋₁₁arylC₁₋₄alkoxy, C₁₋₄alkylthio, hydroxyC₁₋₄alkyl, cyanoC₁₋₄alkyl, C₁₋₄alkoxyC₁₋₄alkyl, C₁₋₄haloalkoxyC₁₋₄alkyl, C₁₋₄alkoxyhaloC₁₋₄alkyl, halohydroxyC₁₋₄alkyl, C₁₋₄alkoxyC₁₋₄alkoxyC₁₋₄alkyl, haloC₁₋₄alkoxyC₁₋₄alkoxyC₁₋₄alkyl, C₁₋₄alkoxyC₁₋₄haloalkoxyC₁₋₄alkyl, C₁₋₄alkoxyC₁₋₄alkoxyC₁₋₄haloalkyl C₁₋₄alkoxyC₁₋₄alkoxy, C₁₋₄alkoxyhaloC₁₋₄alkyl, C₃₋₆cycloalkylC₁₋₄alkyl, C₃₋₆cycloalkylC₁₋₄alkyl substituted by one or more C₁₋₄alkoxy groups in the C₁₋₄alkyl portion, C₃₋₆cycloalkyl, C₃₋₆cycloalkylC₁₋₄alkoxy, C₃₋₆cycloalkylhydroxyC₁₋₄alkyl, C₃₋₆cycloalkylC₁₋₄acyl, C₃₋₆cycloalkylC₁₋₄alkoxyC₁₋₄alkyl, C₃₋₆cycloalkoxyC₁₋₄alkyl, C₁₋₄alkanoyl, C₁₋₄haloalkanoyl, C₁₋₄alkoxycarbonyl, C₁₋₄alkoxycarbonylC₁₋₄alkyl, C₁₋₄alkylsulfonyl, C₁₋₄haloalkylsulfonyl, C₁₋₄alkylsulfinyl, C₁₋₄haloalkylsulfinyl, C₁₋₄alkylsulfonyloxy, C₁₋₄alkylsulfonylC₁₋₄alkyl, C₆₋₁₁arylsulfonyl, C₆₋₁₁arylsulfonyloxy, C₆₋₁₁arylsulfonylC₁₋₄alkyl, C₁₋₄alkylsulfonamido, C₄₋₉heteroarylsulfonyl, C₁₋₄alkylsulfonamidoC₁₋₄alkyl, C₁₋₄alkylamidoC₁₋₄alkyl, C₆₋₁₁arylsulfonamido, C₆₋₁₁arylcarboxamido, C₆₋₁₁arylsulfonamidoC₁₋₄alkyl, C₆₋₁₁arylcarboxamidoC₁₋₄alkyl, C₆₋₁₁aroyl, C₆₋₁₁aroylC₁₋₄alkyl, C₆₋₁₁arylC₁₋₄alkanoyl, formyl, C₁₋₄acyl, haloC₁₋₄acyl, arylC₁₋₄alkoxyC₁₋₄alkyl, C₆₋₁₁aryl, C₆₋₁₁aryl substituted by one to three groups selected from the group consisting of C₁₋₄alkyl, hydroxy, halogen, C₁₋₄alkoxy, C₁₋₄acyl and trifluoromethyl; C₆₋₁₁arylC₁₋₄alkyl, C₃₋₆heterocyclyl, C₃₋₁₀heteroaryl, C₃₋₁₀heteroaryl substituted by one to three C₁₋₄alkyl groups, C₁₋₄alkylaminoC₁₋₄alkyl, a group —NR₉R₁₀, —(CH₂)ₚSO₂NR_{9′}R_{10′}, —NR₉C(O)R₁₀—NHC(O)—C₁₋₂alkyl, —(CH₂)ₚNR_{10′}SO₂R_{9′}, —CR_{9′}═NR_{10′}, —CR_{9′}═NOR_{10′}, —CR_{9′}═C(CN)₂, —CR_{9′}═CH(CN), —(CH₂)ᵣN(R_{9′})C(O)R_{10′}, —(CH₂)ᵣNR₉R₁₀ and —O(CH₂)_{q}NR_{9′}R_{10′}, wherein
each R₉ and R₁₀ is independently halo C₁₋₄alkyl, C₁₋₄alkyl, C₅₋₁₀aryl, or R₉R₁₀ forms part of a C₃₋₆azacyloalkane ring;
each R_{9′} and R_{10′} is independently selected from the group consisting of R₉ and R₁₀ and hydrogen;
each R_{9″} and R_{10″} is independently selected from the group consisting of R_{9′} and R_{10′}, C₁₋₄acyl, haloC₁₋₄acyl, haloaroyl and C₁₋₄alkanoyl;
p is 0, 1, 2, 3 or 4;
q is 2, 3 or 4;
r is 1, 2, 3 or 4;
or Z is selected from the group consisting of:
a monocyclic or bicyclic heteroaryl group, or a bicyclic C₈₋₁₁aryl group which heteroaryl or aryl group is unsubstituted or substituted by one or more groups selected from the group consisting of halogen, hydroxy, oxo, cyano, amino, nitro, C₁₋₆alkyl, C₁₋₄alkoxy, haloC₁₋₄alkyl, haloC₁₋₄alkoxy, C₆₋₁₁arylC₁₋₄alkoxy, C₁₋₄alkylthio, hydroxyC₁₋₄alkyl, C₁₋₄alkoxyC₁₋₄alkyl, C₁₋₄haloalkoxyC₁₋₄alkyl, halohydroxyC₁₋₄alkyl, C₁₋₄alkoxyC₁₋₄alkoxyC₁₋₄alkyl, C₁₋₄alkoxyhaloC₁₋₄alkyl, C₃₋₆cycloalkylC₁₋₄alkyl, C₃₋₆cycloalkylC₁₋₄alkyl substituted by one or more C₁₋₄alkoxy groups in the C₁₋₄alkyl portion, C₃₋₆cycloalkyl, C₃₋₆cycloalkylC₁₋₄alkoxy, C₃₋₆cycloalkylhydroxyC₁₋₄alkyl, C₃₋₆cycloalkylC₁₋₄acyl, C₃₋₆cycloalkylC₁₋₄alkoxyC₁₋₄alkyl, C₃₋₆cycloalkoxyC₁₋₄alkyl, C₁₋₄alkanoyl, C₁₋₄haloalkanoyl, C₁₋₄alkoxycarbonyl, C₁₋₄alkoxycarbonylC₁₋₄alkyl, C₁₋₄alkylsulfonyl, C₁₋₄haloalkylsulfonyl, C₁₋₄alkylsulfinyl, C₁₋₄haloalkylsulfinyl, C₁₋₄alkylsulfonyloxy, C₁₋₄alkylsulfonylC₁₋₄alkyl, C₆₋₁₁arylsulfonyl, C₆₋₁₁arylsulfonyloxy, C₆₋₁₁arylsulfonylC₁₋₄alkyl, C₁₋₄alkylsulfonamido, C₄₋₉heteroarylsulfonyl, C₁₋₄alkylsulfonamidoC₁₋₄alkyl, C₁₋₄alkylamidoC₁₋₄alkyl, C₆₋₁₁arylsulfonamido, C₆₋₁₁arylcarboxamido, C₆₋₁₁arylsulfonamidoC₁₋₄alkyl, C₆₋₁₁arylcarboxamidoC₁₋₄alkyl, C₆₋₁₁aroyl, C₆₋₁₁aroylC₁₋₄alkyl, C₆₋₁₁arylC₁₋₄alkanoyl, formyl, C₁₋₄acyl, haloC₁₋₄acyl, arylC₁₋₄alkoxyC₁₋₄alkyl, C₆₋₁₁aryl, C₆₋₁₁aryl substituted by one to three groups selected from the group consisting of C₁₋₄alkyl, hydroxy, halogen, C₁₋₄alkoxy, C₁₋₄acyl and trifluoromethyl; C₆₋₁₁arylC₁₋₄alkyl, C₄₋₁₀heteroaryl, C₄₋₁₀heteroaryl substituted by one to three C₁₋₄alkyl groups, C₁₋₄alkylaminoC₁₋₄alkyl, a group —NR_{9″}R_{10″}, —(CH₂)ₚCONR₉R₁₀, —(CH₂)ₚSO₂NR_{9′}R_{10′} or —(CH₂)ₚNR_{10′}SO₂R₉, —CR_{9′}═NR_{10′}, —CR_{9′}═NOR_{10′}, —CR_{9′}═C(CN)₂, —CR_{9′}═CH(CN), —(CH₂)_{q}NR_{9′}R_{10′} and —O(CH₂)_{q}NR_{9′}R_{10′} wherein
each R₉ and R₁₀ is independently C₁₋₄alkyl, C₅₋₁₀aryl, or R₉R₁₀ forms part of a C₃₋₆azacyloalkane or C₃₋₆(2-, 3- or 4-oxo)azacycloalkane ring
each R_{9′} and R_{10′} is independently selected from the group consisting of R₉ and R₁₀ and hydrogen;
each R_{9″} and R_{10″} is independently selected from the group consisting of R_{9′} and R_{10′} and C₁₋₄alkanoyl;
p is 0, 1, 2, 3 or 4; and
q is 2, 3 or 4.

19. A compound according to claim 18 wherein X is phenyl, furanyl, thiophenyl or pyridinyl, each of which is unsubstituted or substituted by one or two groups selected from the group consisting of the group consisting of halogen, C₁₋₆alkoxy and cyano.

20. A compound according to claim 18 wherein Y is C₁₋₆alkysulfonyl.

21. A compound according to claim 18 wherein n is 0.

22. A compound according to claim 18 wherein Z is selected from the group consisting of Z′, pyridyl, pyrimidinyl, pyridazinyl, pyrazinyl, triazolyl, triazinyl, pyrrolyl, 1H-pyrrolo[2,3-b]pyridinyl, imidazolyl, thienyl, furanyl, thiadiazolyl, isoxazolyl, isothiazolyl, thiazolyl, oxadiazolyl and oxazolyl, benzothiazolyl, 1,4-benzodioxinyl, 2,3-dihydro-1,4-benzodioxinyl, benzoxazolyl, indolyl, quinolyl, isoquinolinyl, 1-benzopyranyl, 2-benzopyranyl, dihydro-1-benzopyranyl, dihydro-2-benzopyranyl, quinoxalinyl and quinazolinyl.

23. A compound according to claim 18 in which Z is Z′: 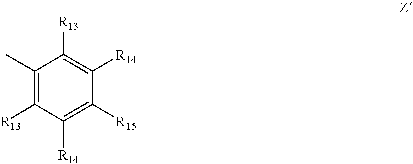
wherein each R₁₃ is independently selected from the group consisting of hydrogen, halogen, formyl and C₁₋₄acyl,
each R₁₄ is independently selected from the group consisting of hydrogen, halogen, C₁₋₆alkyl, C₁₋₄alkoxy, haloC₁₋₄alkyl, haloC₁₋₄alkoxy and —NR_{9″}R_{10″}, wherein R_{9″}R_{10″} forms part of a C₃₋₆azacyloalkane or C₃₋₆(2-, 3- or 4-oxo)azacycloalkane ring;
R₁₅ is selected from the group consisting of hydrogen, halogen, hydroxy, cyano, nitro, C₁₋₆alkyl, C₂₋₄alkenyl, C₁₋₄alkoxy, haloC₁₋₄alkyl, haloC₁₋₄alkoxy, C₆₋₁₁arylC₁₋₄alkoxy, C₁₋₄alkylthio, hydroxyC₁₋₄alkyl, cyanoC₁₋₄alkyl, C₁₋₄alkoxyC₁₋₄alkyl, C₁₋₄haloalkoxyC₁₋₄alkyl, C₁₋₄alkoxyhaloC₁₋₄alkyl, C₁₋₄alkanoyl, haloC₁₋₄alkanoyl, halohydroxyC₁₋₄alkyl, C₁₋₄alkoxyC₁₋₄alkoxyC₁₋₄alkyl, C₁₋₄alkoxyC₁₋₄alkoxy, C₁₋₄alkoxyhaloC₁₋₄alkyl, haloC₁₋₄alkoxyC₁₋₄alkoxyC₁₋₄alkyl, C₁₋₄alkoxyC₁₋₄haloalkoxyC₁₋₄alkyl, C₁₋₄alkoxyC₁₋₄alkoxyC₁₋₄haloalkyl, C₃₋₆cycloalkylC₁₋₄alkyl substituted by one or more C₁₋₄alkoxy groups in the C₁₋₄alkyl portion, C₃₋₆cycloalkylC₁₋₄alkoxy, C₃₋₆cycloalkylhydroxyC₁₋₄alkyl, C₃₋₆cycloalkylC₁₋₄alkoxyC₁₋₄alkyl, C₃₋₆cycloalkoxyC₁₋₄alkyl, C₁₋₄alkoxycarbonyl, C₁₋₄alkylsulfonyl, C₁₋₄alkylsulfinyl, C₁₋₄haloalkylsulfinyl, formyl, C₁₋₄acyl, haloC₁₋₄acyl, arylC₁₋₄alkoxyC₁₋₄alkyl, C₆₋₁₁aryl substituted by one to three C₁₋₄alkyl groups, C₃₋₆heterocyclyl, C₃₋₁₀heteroaryl, C₃₋₁₀heteroaryl substituted by one to three C₁₋₄alkyl groups, a group —NR₉R₁₀, —NR₉C(O)R₁₀, —NHC(O)—C₁₋₂alkyl, —CR_{9′}═NOR_{10′}, —(CH₂)ᵣN(R_{9′})C(O)R_{10′}, —(CH₂)ᵣNR₉R₁₀ and —O(CH₂)_{q}NR_{9′}R_{10′}, wherein
each R₉ and R₁₀ is independently halo C₁₋₄alkyl, C₁₋₄alkyl, C₅₋₁₀aryl, or R₉R₁₀ forms part of a C₃₋₆azacyloalkane ring
each R_{9′} and R_{10′} is independently selected from the group consisting of R₉ and R₁₀ and hydrogen;
each R_{9″} and R_{10″} is independently selected from the group consisting of R_{9′} and R_{10′}, C₁₋₄acyl, haloC₁₋₄acyl, haloaroyl and C₁₋₄alkanoyl;
q is 2, 3 or 4; and
r is 1, 2, 3 or 4.

24. A compound claimed in claim 18 in which R₁₅ is C₁₋₄alkoxyC₁₋₄alkyl, haloC₁₋₄alkoxyC₁₋₄alkyl, C₁₋₄alkoxyhaloC₁₋₄alkyl, C₁₋₄alkanoyl, haloC₁₋₄alkanoyl, C₁₋₄alkoxyC₁₋₄alkoxyC₁₋₄alkyl, haloC₁₋₄alkoxyC₁₋₄alkoxyC₁₋₄alkyl, C₁₋₄alkoxyC₁₋₄haloalkoxyC₁₋₄alkyl, C₁₋₄alkoxyC₁₋₄alkoxyC₁₋₄haloalkyl, haloC₁₋₄alkyl (for example trifluoromethyl) or NR_{9′}R_{10′} wherein:
R_{9′} is C₁₋₄alkyl or haloC₁₋₄alkyl; and
R_{10′} is C₁₋₄ acyl, haloC₁₋₄ acyl or haloC₅₋₁₁aroyl.

25. A compound as claimed in claim 18, which is any one of Examples 1, 2, 4, 6, 7, 8, 10, 12 to 48 or 50 to 131 or a salt or solvate thereof.

26. A method of preparing a compound of formula (I) as defined in claim 18 or a salt thereof, comprising the step of:
(a) reacting a compound of formula (II): 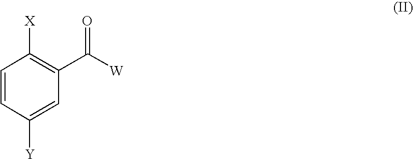
wherein X and Y are as defined in claim 18 and W is —OH or —Cl, with a compound of formula (III): 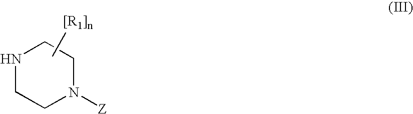
wherein Z, n and R₁ are as defined in claim 18; or
(b) reacting a compound of formula (IV): 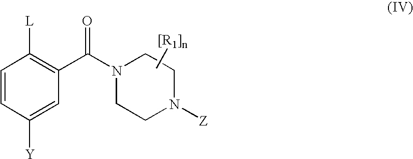
wherein Y, R₁, n and Z are as defined in claim 18 and L is a leaving group such as chloride, bromide or trifluoromethanesulfonate, with a compound of formula (V):
XB(OH)₂ (V)
wherein X is as defined in claim 18;
and thereafter optionally for step (a) or step (b):
removing any protecting groups and/or
converting a compound of formula (I) into another compound of formula (I) and/or
forming a salt or solvate.

27. A pharmaceutical composition comprising a compound as defined in claim 18 and at least one pharmaceutically acceptable carrier, diluent or excipient.

28. A method of treating a schizophrenia comprising administering to a human in need thereof an effective amount of a compound according to claim 18 or a salt thereof.
